(19) United States
(12) Patent Application Publication
(10) Pub. No.: US 2015/0157700 A1
(43) Pub. Date: Jun. 11, 2015

(54) ADENOVIRAL VECTOR-BASED MALARIA VACCINE

(71) Applicant: GanVec, Inc., Gaithersburg, MD (US)

(72) Inventors: Joseph T. Bruder, Gaithersburg, MD (US); Jason G.D. Gall, Germantown, MD (US); Duncan McVey, Derwood, MD (US); Douglas E. Brough, Gaithersburg, MD (US)

(21) Appl. No.: 14/373,574

(22) PCT Filed: Feb. 1, 2013
(86) PCT No.: PCT/US2013/024262

§ 371 Date: Jul. 21, 2014

Related U.S. Application Data
(60) Provisional application No. 61/593,953, filed on Feb. 2, 2012.
(60) Provisional application No. 61/593,964, filed on Feb. 2, 2012.
(60) Provisional application No. 61/593,980, filed on Feb. 2, 2012.

(51) Int. Cl. A61K 39/015; C12N 7/00

(52) U.S. Cl. CPC A61K 39/015; C12N 7/00; A61K 2039/5256

(57) ABSTRACT

The invention provides an adenovirus or adenoviral vector characterized by comprising a nucleic acid sequence encoding one or more Plasmodium antigens and one or more particular nucleic acid sequences or one or more particular amino acid sequences, or portions thereof, pertaining to, for example, an adenoviral pIX protein, DNA polymerase protein, penton protein, hexon protein, and/or fiber protein, as well as a method of inducing an immune response against Plasmodium falciparum in a mammal by administering the adenovirus or adenoviral vector to the mammal.

### CROSS-REFERENCE TO RELATED APPLICATIONS

This patent application claims the benefit of U.S. Provisional Patent Application No. 61/593,953, filed Feb. 2, 2012, U.S. Provisional Patent Application No. 61/593,964, filed Feb. 2, 2012, and U.S. Provisional Patent Application No. 61/593,980, filed Feb. 2, 2012, the disclosures of which are incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 1,111,510 Byte ASCII (Text) file named “712147_ST25.TXT,” created on Jan. 28, 2013.

### BACKGROUND OF THE INVENTION

Malaria is one of the most devastating parasitic diseases affecting humans. Indeed, 41% of the world's population lives in areas where malaria is transmitted (e.g., parts of Africa, Asia, the Middle East, Central and South America, Hispaniola, and Oceania). The World Health Organization (WHO) and the Centers for Disease Control (CDC) estimate that malaria infects 300-500 million people and kills 700,000-3 million people annually, with the majority of deaths occurring in children in sub-Saharan Africa. Malaria also is a major health concern to U.S. military personnel deployed to tropical regions of the world. For example, in August 2003, 28% of the 26^{th} Marine Expeditionary Unit and Joint Task Force briefly deployed to Monrovia, Liberia, were infected with the malaria parasite Plasmodium falciparum. In addition, one 157-man Marine Expeditionary Unit sustained a 44% malaria casualty rate over a 12-day period while stationed at Robert International Airport in Monrovia. In all conflicts during the past century conducted in malaria endemic areas, malaria has been the leading cause of casualties, exceeding enemy-inflicted casualties in its impact on “person-days” lost from duty.

To combat malaria during U.S military operations, preventive drugs, insect repellants, and barriers have been used with some success, but developing drug resistance by the malaria parasite and insecticide resistance by mosquito vectors has limited the efficacy of these agents. Moreover, the logistical burden and side effects associated with the use of these agents often is associated with high non-compliance rates. Vaccines are the most cost effective and efficient therapeutic interventions for infectious diseases. In this regard, vaccination has the advantage of administration prior to military deployment and likely reduction in non-compliance risks. However, decades of research and development directed to a malaria vaccine have not proven successful. Recent efforts have focused on developing vaccines against several specific malaria genes and delivery vector systems including adenovirus, poxvirus, and plasmids. Malaria vaccine development and related clinical trials are reviewed in, for example, Graves and Gelband, Cochrane Database Syst. Rev., 1: CD000129 (2003), Moore et al., Lancet Infect. Dis., 2: 737-743 (2002), Carvalho et al., Scand. J. Immunol., 56: 327-343 (2002), Moorthy and Hill, Br. Med. Bull., 62: 59-72 (2002), Greenwood and Alonso, Chem. Immunol., 80: 366-395 (2002), and Richie and Saul, Nature, 415: 694-701 (2002).

Over the past 15-20 years, a series of Phase 1/2 vaccine trials have been reported using synthetic peptides or recombinant proteins based on malarial antigens. Most of these trials have been directed against the sporozoite stage or liver stage of the Plasmodium life cycle, where the use of experimental mosquito challenges allows rapid progress through Phase 1 to Phase 2a preliminary efficacy studies. Anti-sporozoite vaccines tested include completely synthetic peptides, conjugates of synthetic peptide with proteins such as tetanus toxoid (to provide T cell help), recombinant malaria proteins, particle-forming recombinant chimeric constructs, recombinant viruses, and bacteria and DNA vaccines. Several trials of asexual blood stage vaccines have used either synthetic peptide conjugates or recombinant proteins. There also has been a single trial of a transmission blocking vaccine (recombinant Pfs25). A recurring problem identified in all of these vaccination strategies is the difficulty in obtaining a sufficiently strong and long lasting immune response in humans, despite the strong immunogenic response in animal models.

To overcome these limitations, the development of potent immune-stimulatory conjugates or adjuvants to boost the human response has been explored, in addition to the development of vaccines directed against the circumsporozoite protein (CSP), the principal sporozoite coat protein. Anti-CSP vaccines using recombinant proteins, peptide conjugates, recombinant protein conjugates, and chimeric proteins have been shown to elicit anti-CSP antibodies. Although considerable efforts are still being directed at the development of protein-based vaccines, alternative technologies such as DNA and viral based vaccines have shown some promise with regard to immunogenicity and protective efficacy, at least in animal models.

In this regard, DNA vaccines encoding Plasmodium antigens have been developed and can induce CD8+ CTL and IFN-γ responses, as well as protection against sporozoite challenge in mice (see Sedegah et al., Proc. Natl. Acad. Sci. USA, 91: 9866-9870 (1994), and Doolan et al., J. Exp. Med., 183: 1739-1746 (1996)) and monkeys (Wang et al., Science, 282: 476-480 (1998), Rogers et al., Infect. Immun., 69: 5565-5572 (2001), and Rogers et al., Infect. Immun., 70: 4329-4335 (2002)). Furthermore, Phase I and Phase 2a clinical trials have established the safety, tolerability, and immunogenicity of DNA vaccines encoding malaria antigens in normal healthy humans (see, e.g., Wang et al., Infect Immun., 66: 4193-41202 (1998), Le et al., Vaccine, 18: 1893-1901 (2000), and Epstein et al., Hum. Gene Ther., 13: 1551-1560 (2002)). However, the immunogenicity of first and second-generation DNA vaccines in nonhuman primates and in humans has been suboptimal. Even in murine models, DNA vaccines are not effective at activating both arms of the immune system (see, e.g., Doolan et al., supra, Sedegah et al., supra, Sedegah et al., Proc. Natl. Acad. Sci. USA, 95: 7648-7653 (1998), Zavala et al., Virology, 280: 155-159 (2001), and Pardoll, Nat. Rev. Immunol, 2: 227-238 (2002)).

Thus, there remains a need for constructs that can be used in methods to effectively prevent and/or treat malaria. The invention provides such constructs and methods.

### BRIEF SUMMARY OF THE INVENTION

The invention provides an adenovirus or adenoviral vector. The adenovirus or adenoviral vector comprises (1) a nucleic acid sequence encoding one or more Plasmodium antigens and (2) one or more of the nucleic acid sequences selected from the group consisting of (a) the nucleic acid sequence of SEQ ID NO: 1, (b) a nucleic acid sequence that is at least 98.5% identical to SEQ ID NO: 2, (c) a nucleic acid sequence that is at least 90% identical to SEQ ID NO: 3, (d) a nucleic acid sequence that is at least 80% identical to SEQ ID NO: 4, and (e) a nucleic acid sequence that is at least 89% identical to SEQ ID NO: 5.

The invention provides an adenovirus or adenoviral vector comprising (1) a nucleic acid sequence encoding one or more Plasmodium antigens and (2) one or more of the nucleic acid sequences selected from the group consisting of (a) a nucleic acid sequence that is at least 98.6% identical to SEQ ID NO: 6, (b) a nucleic acid sequence that is at least 99.06% identical to SEQ ID NO: 7, (c) a nucleic acid sequence that is at least 97.13% identical to SEQ ID NO: 8, (d) a nucleic acid sequence that is at least 90.7% identical to SEQ ID NO: 9, and (e) a nucleic acid sequence that is at least 96.6% identical to SEQ ID NO: 10.

The invention provides an adenovirus or adenoviral vector comprising (1) a nucleic acid sequence encoding one or more Plasmodium antigens and (2) one or more of the nucleic acid sequences selected from the group consisting of (a) a nucleic acid sequence comprising at least 121 contiguous nucleotides of SEQ ID NO: 6, (b) a nucleic acid sequence comprising at least 462 contiguous nucleotides of SEQ ID NO: 7, (c) a nucleic acid sequence comprising at least 234 contiguous nucleotides of SEQ ID NO: 8, (d) a nucleic acid sequence comprising at least 606 contiguous nucleotides of SEQ ID NO: 9, and (e) a nucleic acid sequence comprising at least 188 contiguous nucleotides of SEQ ID NO: 10.

The invention provides an adenovirus or adenoviral vector comprising (1) a nucleic acid sequence encoding one or more Plasmodium antigens and (2) one or more of the amino acid sequences selected from the group consisting of (a) the amino acid sequence of SEQ ID NO: 11, (b) an amino acid sequence that is at least 82% identical to SEQ ID NO: 13, (c) an amino acid sequence that is at least 80% identical to SEQ ID NO: 14, and (d) an amino acid sequence that is at least 83% identical to SEQ ID NO: 15.

The invention provides an adenovirus or adenoviral vector comprising (1) a nucleic acid sequence encoding one or more Plasmodium antigens and (2) one or more of the nucleic acid sequences selected from the group consisting of (a) a nucleic acid sequence encoding the amino acid sequence of SEQ ID NO: 11, (b) a nucleic acid sequence encoding an amino acid sequence that is at least 99% identical to SEQ ID NO: 12, (c) a nucleic acid sequence encoding an amino acid sequence that is at least 82% identical to SEQ ID NO: 13, (d) a nucleic acid sequence encoding an amino acid sequence that is at least 80% identical to SEQ ID NO: 14, and (e) a nucleic acid sequence encoding an amino acid sequence that is at least 83% identical to SEQ ID NO: 15.

The invention provides an adenovirus or adenoviral vector comprising (1) a nucleic acid sequence encoding one or more Plasmodium antigens and (2) one or more of the amino acid sequences selected from the group consisting of (a) the amino acid sequence of SEQ ID NO: 16, (b) an amino acid sequence that is at least 97.8% identical to SEQ ID NO: 18, (c) an amino acid that is at least 93.4% identical to SEQ ID NO: 19, and (d) an amino acid sequence that is at least 98.2% identical to SEQ ID NO: 20.

The invention provides an adenovirus or adenoviral vector comprising (1) a nucleic acid sequence encoding one or more Plasmodium antigens and (2) one or more of the nucleic acid sequences selected from the group consisting of (a) a nucleic acid sequence encoding the amino acid sequence of SEQ ID NO: 16, (b) a nucleic acid sequence encoding an amino acid sequence that is at least 99.78% identical to SEQ ID NO: 17, (c) a nucleic acid sequence encoding an amino acid sequence that is at least 97.8% identical to SEQ ID NO: 18, (d) a nucleic acid sequence encoding an amino acid that is at least 93.4% identical to SEQ ID NO: 19, and (e) a nucleic acid sequence encoding an amino acid sequence that is at least 98.2% identical to SEQ ID NO: 20.

The invention provides an adenovirus or adenoviral vector comprising (1) a nucleic acid sequence encoding one or more Plasmodium antigens and (2) one or more of the amino acid sequences selected from the group consisting of (a) an amino acid sequence comprising at least 89 contiguous amino acid residues of SEQ ID NO: 16, (b) an amino acid sequence comprising at least 247 contiguous amino acid residues of SEQ ID NO: 18, (c) an amino acid sequence comprising at least 370 contiguous amino acid residues of SEQ ID NO: 19, and (d) an amino acid sequence comprising at least 192 contiguous amino acid residues of SEQ ID NO: 20.

The invention provides an adenovirus or adenoviral vector comprising (1) a nucleic acid sequence encoding one or more Plasmodium antigens and (2) one or more of the nucleic acid sequences selected from the group consisting of (a) a nucleic acid sequence encoding an amino acid sequence comprising at least 89 contiguous amino acid residues of SEQ ID NO: 16, (b) a nucleic acid sequence encoding an amino acid sequence comprising at least 428 contiguous amino acid residues of SEQ ID NO: 17, (c) a nucleic acid sequence encoding an amino acid sequence comprising at least 247 contiguous amino acid residues of SEQ ID NO: 18, (d) a nucleic acid sequence encoding an amino acid sequence comprising at least 370 contiguous amino acid residues of SEQ ID NO: 19, and (e) a nucleic acid sequence encoding an amino acid sequence comprising at least 192 contiguous amino acid residues of SEQ ID NO: 20.

The invention provides an adenovirus or adenoviral vector comprising (1) a nucleic acid sequence encoding one or more Plasmodium antigens and (2) one or more of the nucleic acid sequences selected from the group consisting of (a) a nucleic acid sequence that is at least 96% identical to SEQ ID NO: 25, (b) a nucleic acid sequence that is at least 99% identical to SEQ ID NO: 26, (c) a nucleic acid sequence that is at least 80% identical to SEQ ID NO: 27, (d) a nucleic acid sequence that is at least 80% identical to SEQ ID NO: 28, and (e) a nucleic acid sequence that is at least 85.4% identical to SEQ ID NO: 29.

The invention provides an adenovirus or adenoviral vector comprising (1) a nucleic acid sequence encoding one or more Plasmodium antigens and (2) one or more of the nucleic acid sequences selected from the group consisting of (a) a nucleic acid sequence that is at least 98.7% identical to SEQ ID NO: 30, (b) a nucleic acid sequence that is at least 98.9% identical to SEQ ID NO: 31, (c) a nucleic acid sequence that is at least 99.4% identical to SEQ ID NO: 32, (d) a nucleic acid sequence that is at least 99.1% identical to SEQ ID NO: 33, (e) a nucleic acid sequence that is at least 81.25% identical to SEQ ID NO: 34, (f) a nucleic acid sequence that is at least 90.83% identical to SEQ ID NO: 35, and (g) a nucleic acid sequence that is at least 82.5% identical to SEQ ID NO: 36.

The invention provides an adenovirus or adenoviral vector comprising (1) a nucleic acid sequence encoding one or more Plasmodium antigens and (2) one or more of the nucleic acid sequences selected from the group consisting of (a) a nucleic acid sequence comprising at least 162 contiguous nucleotides of SEQ ID NO: 30, (b) a nucleic acid sequence comprising at least 162 contiguous nucleotides of SEQ ID NO: 31, (c) a nucleic acid sequence comprising at least 121 contiguous nucleotides of SEQ ID NO: 32, (d) a nucleic acid sequence comprising at least 531 contiguous nucleotides of SEQ ID NO: 33, (e) a nucleic acid sequence comprising at least 156 contiguous nucleotides of SEQ ID NO: 34, (f) a nucleic acid sequence comprising at least 192 contiguous nucleotides of SEQ ID NO: 35, and (g) a nucleic acid sequence comprising at least 84 contiguous nucleotides of SEQ ID NO: 36.

The invention provides an adenovirus or adenoviral vector comprising (1) a nucleic acid sequence encoding one or more Plasmodium antigens and (2) one or more of the amino acid sequences selected from the group consisting of (a) an amino acid sequence that is at least 85% identical to SEQ ID NO: 38, (b) an amino acid sequence that is at least 80% identical to SEQ ID NO: 39, and (c) an amino acid sequence that is at least 80% identical to SEQ ID NO: 40.

The invention provides an adenovirus or adenoviral vector comprising (1) a nucleic acid sequence encoding one or more Plasmodium antigens and (2) one or more of the nucleic acid sequences selected from the group consisting of (a) a nucleic acid sequence encoding an amino acid sequence that is at least 99.7% identical to SEQ ID NO: 37, (b) a nucleic acid sequence encoding an amino acid sequence that is at least 85% identical to SEQ ID NO: 38, (c) a nucleic acid sequence encoding an amino acid sequence that is at least 80% identical to SEQ ID NO: 39, and (d) a nucleic acid sequence encoding an amino acid sequence that is at least 80% identical to SEQ ID NO: 40.

The invention provides an adenovirus or adenoviral vector comprising (1) a nucleic acid sequence encoding one or more Plasmodium antigens and (2) one or more of the amino acid sequences selected from the group consisting of (a) an amino acid sequence that is at least 99% identical to SEQ ID NO: 42, (b) an amino acid sequence that is at least 81.4% identical to SEQ ID NO: 44, (c) an amino acid sequence that is at least 91.3% identical to SEQ ID NO: 45, and (d) an amino acid sequence that is at least 83.4% identical to SEQ ID NO: 46.

The invention provides an adenovirus or adenoviral vector comprising (1) a nucleic acid sequence encoding one or more Plasmodium antigens and (2) one or more of the nucleic acid sequences selected from the group consisting of (a) a nucleic acid sequence encoding an amino acid sequence that is at least 99% identical to SEQ ID NO: 42, (b) a nucleic acid sequence encoding an amino acid sequence that is at least 99.75% identical to SEQ ID NO: 43, (c) a nucleic acid sequence encoding an amino acid sequence that is at least 81.4% identical to SEQ ID NO: 44, (d) a nucleic acid sequence encoding an amino acid sequence that is at least 91.3% identical to SEQ ID NO: 45, (e) a nucleic acid sequence encoding an amino acid sequence that is at least 83.4% identical to SEQ ID NO: 46.

The invention provides an adenovirus or adenoviral vector comprising (1) a nucleic acid sequence encoding one or more Plasmodium antigens and (2) one or more of the amino acid sequences selected from the group consisting of (a) an amino acid sequence comprising at least 89 contiguous amino acid residues of SEQ ID NO: 42, (b) an amino acid sequence comprising at least 114 contiguous amino acid residues of SEQ ID NO: 44, (c) an amino acid sequence comprising at least 231 contiguous amino acid residues of SEQ ID NO: 45, and (d) an amino acid sequence comprising at least 30 contiguous amino acid residues of SEQ ID NO: 46.

The invention provides an adenovirus or adenoviral vector comprising (1) a nucleic acid sequence encoding one or more Plasmodium antigens and (2) one or more of the nucleic acid sequences selected from the group consisting of (a) a nucleic acid sequence encoding an amino acid sequence comprising at least 89 contiguous amino acid residues of SEQ ID NO: 42, (b) a nucleic acid sequence encoding an amino acid sequence comprising at least 428 contiguous amino acid residues of SEQ ID NO: 43, (c) a nucleic acid sequence encoding an amino acid sequence comprising at least 114 contiguous amino acid residues of SEQ ID NO: 44, (d) a nucleic acid sequence encoding an amino acid sequence comprising at least 231 contiguous amino acid residues of SEQ ID NO: 45, and (e) a nucleic acid sequence encoding an amino acid sequence comprising at least 30 contiguous amino acid residues of SEQ ID NO: 46.

The invention provides an adenovirus or adenoviral vector comprising (1) a nucleic acid sequence encoding one or more Plasmodium antigens and (2) one or more of the nucleic acid sequences selected from the group consisting of (a) a nucleic acid sequence that is at least 97% identical to SEQ ID NO: 59, (b) a nucleic acid sequence that is at least 97.5% identical to SEQ ID NO: 60, (c) a nucleic acid sequence that is at least 80% identical to SEQ ID NO: 61, (d) a nucleic acid sequence that is at least 96% identical to SEQ ID NO: 62, and (e) a nucleic acid sequence that is at least 96% identical to SEQ ID NO: 63.

The invention provides an adenovirus or adenoviral vector comprising (1) a nucleic acid sequence encoding one or more Plasmodium antigens and (2) one or more of the nucleic acid sequences selected from the group consisting of (a) a nucleic acid sequence that is at least 98.4% identical to SEQ ID NO: 64, (b) a nucleic acid sequence that is at least 99.01% identical to SEQ ID NO: 65, (c) a nucleic acid sequence that is at least 97.08% identical to SEQ ID NO: 66, (d) a nucleic acid sequence that is at least 96.52% identical to SEQ ID NO: 67, and (e) a nucleic acid sequence that is at least 98.49% identical to SEQ ID NO: 68.

The invention provides an adenovirus or adenoviral vector comprising (1) a nucleic acid sequence encoding one or more Plasmodium antigens and (2) one or more of the nucleic acid sequences selected from the group consisting of (a) a nucleic acid sequence comprising at least 121 contiguous nucleotides of SEQ ID NO: 64, (b) a nucleic acid sequence comprising at least 462 contiguous nucleotides of SEQ ID NO: 65, (c) a nucleic acid sequence comprising at least 234 contiguous nucleotides of SEQ ID NO: 66, (d) a nucleic acid sequence comprising at least 606 contiguous nucleotides of SEQ ID NO: 67, and (e) a nucleic acid sequence comprising at least 188 contiguous nucleotides of SEQ ID NO: 68.

The invention provides an adenovirus or adenoviral vector comprising (1) a nucleic acid sequence encoding one or more Plasmodium antigens and (2) one or more of the amino acid sequences selected from the group consisting of (a) an amino acid sequence that is at least 93% identical to SEQ ID NO: 69, (b) an amino acid sequence that is at least 80% identical to SEQ ID NO: 71, (c) an amino acid sequence that is at least 92% identical to SEQ ID NO: 72, and (d) an amino acid sequence that is at least 88% identical to SEQ ID NO: 73.

The invention provides an adenovirus or adenoviral vector comprising (1) a nucleic acid sequence encoding one or more Plasmodium antigens and (2) one or more of the nucleic acid sequences selected from the group consisting of (a) a nucleic acid sequence encoding an amino acid sequence that is at least 93% identical to SEQ ID NO: 69, (b) a nucleic acid sequence encoding an amino acid sequence that is at least 95% identical to SEQ ID NO: 70, (c) a nucleic acid sequence encoding an amino acid sequence that is at least 80% identical to SEQ ID NO: 71, (d) a nucleic acid sequence encoding an amino acid sequence that is at least 92% identical to SEQ ID NO: 72, and (e) a nucleic acid sequence encoding an amino acid sequence that is at least 88% identical to SEQ ID NO: 73.

The invention provides an adenovirus or adenoviral vector comprising (1) a nucleic acid sequence encoding one or more Plasmodium antigens and (2) one or more of the amino acid sequences selected from the group consisting of (a) an amino acid sequence that is at least 99% identical to SEQ ID NO: 74, (b) an amino acid sequence that is at least 97.8% identical to SEQ ID NO: 76, (c) an amino acid sequence that is at least 99.1% identical to SEQ ID NO: 77, and (d) an amino acid sequence that is at least 99.2% identical to SEQ ID NO: 78.

The invention provides an adenovirus or adenoviral vector comprising (1) a nucleic acid sequence encoding one or more Plasmodium antigens and (2) one or more of the nucleic acid sequences selected from the group consisting of (a) a nucleic acid sequence encoding an amino acid sequence that is at least 99% identical to SEQ ID NO: 74, (b) a nucleic acid sequence encoding an amino acid sequence that is at least 99.5% identical to SEQ ID NO: 75, (c) a nucleic acid sequence encoding an amino acid sequence that is at least 97.8% identical to SEQ ID NO: 76, (d) a nucleic acid sequence encoding an amino acid sequence that is at least 99.1% identical to SEQ ID NO: 77, and (e) a nucleic acid sequence encoding an amino acid sequence that is at least 99.2% identical to SEQ ID NO: 78.

The invention provides an adenovirus or adenoviral vector comprising (1) a nucleic acid sequence encoding one or more Plasmodium antigens and (2) one or more of the amino acid sequences selected from the group consisting of (a) an amino acid sequence comprising at least 89 contiguous amino acid residues of SEQ ID NO: 74, (b) an amino acid sequence comprising at least 247 contiguous amino acid residues of SEQ ID NO: 76, (c) an amino acid sequence comprising at least 230 contiguous amino acid residues of SEQ ID NO: 77, and (d) an amino acid sequence comprising at least 231 contiguous amino acid residues of SEQ ID NO: 78.

The invention provides an adenovirus or adenoviral vector comprising (1) a nucleic acid sequence encoding one or more Plasmodium antigens and (2) one or more of the nucleic acid sequences selected from the group consisting of (a) a nucleic acid sequence encoding an amino acid sequence comprising at least 89 contiguous amino acid residues of SEQ ID NO: 74, (b) a nucleic acid sequence encoding an amino acid sequence comprising at least 286 contiguous amino acid residues of SEQ ID NO: 75, (c) a nucleic acid sequence encoding an amino acid sequence comprising at least 247 contiguous amino acid residues of SEQ ID NO: 76, (d) a nucleic acid sequence encoding an amino acid sequence comprising at least 230 contiguous amino acid residues of SEQ ID NO: 77, and (e) a nucleic acid sequence encoding an amino acid sequence comprising at least 231 contiguous amino acid residues of SEQ ID NO: 78.

The invention provides a method of inducing an immune response against Plasmodium falciparum in a mammal, especially a human, which comprises administering to the mammal an aforementioned inventive adenovirus or adenoviral vector, whereupon the nucleic acid sequence encoding the Plasmodium antigen is expressed in the mammal to produce the Plasmodium antigen and thereby induce an immune response against Plasmodium falciparum in the mammal.

### BRIEF DESCRIPTION OF THE SEVERAL VIEWS OF THE DRAWING(S)

FIG. 1 is a graph which depicts experimental data illustrating that immunization of mice with an adenoviral vector encoding P. yoelii CSP (GC44PyCSP.o) induces a CD8+ T cell response.

FIG. 2 is a graph which depicts experimental data illustrating that immunization of mice with an adenoviral vector encoding P. yoelii CSP (GC45PyCSP.o) induces a CD8+ T cell response.

FIG. 3 is a graph which depicts experimental data illustrating that immunization of mice with an adenoviral vector encoding P. yoelii CSP (GC46PyCSP.o) induces a CD8+ T cell response.

FIG. 4 is a graph which depicts experimental data illustrating that immunization of mice with an adenoviral vector encoding P. yoelii CSP (GC44PyCSP.o) induces CSP-specific antibodies.

FIG. 5 is a graph which depicts experimental data illustrating that immunization of mice with an adenoviral vector encoding P. yoelii CSP (GC45PyCSP.o) induces CSP-specific antibodies.

FIG. 6 is a graph which depicts experimental data illustrating that immunization of mice with an adenoviral vector encoding P. yoelii CSP (GC46PyCSP.o) induces CSP-specific antibodies.

### DETAILED DESCRIPTION OF THE INVENTION

Adenoviruses are generally associated with benign pathologies in humans, and the genomes of adenoviruses isolated from a variety of species, including humans, have been extensively studied. Adenovirus is a medium-sized (90-100 nm), nonenveloped icosohedral virus containing approximately 36 kb of double-stranded DNA. The adenovirus capsid mediates the key interactions of the early stages of the infection of a cell by the virus, and is required for packaging adenovirus genomes at the end of the adenovirus life cycle. The capsid comprises 252 capsomeres, which includes 240 hexons, 12 penton base proteins, and 12 fibers (Ginsberg et al., Virology, 28: 782-83 (1966)). The hexon comprises three identical proteins, namely polypeptide II (Roberts et al., Science, 232: 1148-51 (1986)). The penton base comprises five identical proteins and the fiber comprises three identical proteins. Proteins Ilia, VI, and IX are present in the adenoviral coat and are believed to stabilize the viral capsid (Stewart et al., Cell, 67: 145-54 (1991), and Stewart et al., EMBO J., 12(7): 2589-99 (1993)). The expression of the capsid proteins, with the exception of pIX, is dependent on the adenovirus polymerase protein. Therefore, major components of an adenovirus particle are expressed from the genome only when the polymerase protein gene is present and expressed.

Several features of adenoviruses make them ideal vehicles for transferring genetic material to cells for therapeutic applications (i.e. “gene therapy”), or for use as antigen delivery systems for vaccine applications. For example, adenoviruses can be produced in high titers (e.g., about 10¹³ particle units (pu)), and can transfer genetic material to nonreplicating and replicating cells. The adenoviral genome can be manipulated to carry a large amount of exogenous DNA (up to about 8 kb), and the adenoviral capsid can potentiate the transfer of even longer sequences (Curiel et al., Hum. Gene Ther., 3: 147-154 (1992)). Additionally, adenoviruses generally do not integrate into the host cell chromosome, but rather are maintained as a linear episome, thereby minimizing the likelihood that a recombinant adenovirus will interfere with normal cell function.

The invention is predicated, at least in part, on the discovery and isolation of an adenovirus that has not previously been identified or isolated. The adenovirus described herein was isolated from a gorilla. There are four widely recognized gorilla subspecies within the two species of Eastern Gorilla (Gorilla beringei) and Western Gorilla (Gorilla gorilla). The Western Gorilla species includes the subspecies Western Lowland Gorilla (Gorilla gorilla gorilla) and Cross River Gorilla (Gorilla gorilla diehli). The Eastern Gorilla species includes the subspecies Mountain Gorilla (Gorilla beringei beringei) and Eastern Lowland Gorilla (Gorilla beringei graueri) (see, e.g., Wilson and Reeder, eds., Mammalian Species of the World, 3^{rd} ed., Johns Hopkins University Press, Baltimore, Md. (2005)). The adenovirus of the invention can be isolated from Western Lowland Gorilla (Gorilla gorilla gorilla), Cross River Gorilla (Gorilla gorilla diehli), Mountain Gorilla (Gorilla beringei beringei), or Eastern Lowland Gorilla (Gorilla beringei graueri).

The genomes of several such adenoviruses have been analyzed, and it has been determined that the adenovirus can have the nucleic acid sequence of, for example, SEQ ID NO: 21, SEQ ID NO: 22, SEQ ID NO: 23, SEQ ID NO: 24, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 79, SEQ ID NO: 80, SEQ ID NO: 81, or SEQ ID NO: 82, each of which includes a number of sub-sequences that serve to uniquely define the adenovirus, namely the nucleic acid sequences of SEQ ID NOs: 1-10, 25-36, and 59-68, and the amino acid sequences of SEQ ID NOs: 11-20, 37-46, and 69-78. SEQ ID NOs: 6-10 encode the amino acid sequences of SEQ ID NOs: 16-20, respectively. SEQ ID NOs: 1-5 are a subset of the nucleic acid sequences of SEQ ID NOs: 6-10, respectively. SEQ ID NOs: 11-15 are a subset of the amino acid sequences of SEQ ID NOs: 16-20, respectively. SEQ ID NOs: 30-36 encode the amino acid sequences of SEQ ID NOs: 41-46, respectively. SEQ ID NOs: 25-29 are a subset of the nucleic acid sequences of SEQ ID NOs: 30 and 33-36, respectively. SEQ ID NOs: 37-40 are a subset of the amino acid sequences of SEQ ID NOs: 43-46, respectively. SEQ ID NOs: 64-68 encode the amino acid sequences of SEQ ID NOs: 74-78, respectively. SEQ ID NOs: 59-63 are a subset of the nucleic acid sequences of SEQ ID NOs: 64-68, respectively. SEQ ID NOs: 69-73 are a subset of the amino acid sequences of SEQ ID NOs: 74-78, respectively.

The adenovirus can be modified in the same manner as previously known adenoviruses to be used as an adenoviral vector, e.g., a gene delivery vehicle.

The term “adenovirus,” as used herein, refers to an adenovirus that retains the ability to participate in the adenovirus life cycle and has not been physically inactivated by, for example, disruption (e.g., sonication), denaturing (e.g., using heat or solvents), or cross-linkage (e.g., via formalin cross-linking). The “adenovirus life cycle” includes (1) virus binding and entry into cells, (2) transcription of the adenoviral genome and translation of adenovirus proteins, (3) replication of the adenoviral genome, and (4) viral particle assembly (see, e.g., Fields Virology, 5^{th} ed., Knipe et al. (eds.), Lippincott, Williams & Wilkins, Philadelphia (2006)).

The term “adenoviral vector,” as used herein, refers to an adenovirus in which the adenoviral genome has been manipulated to accommodate one or more nucleic acid sequences that are non-native with respect to the adenoviral genome. Typically, an adenoviral vector is generated try introducing one or more mutations (e.g., a deletion, insertion, or substitution) into the adenoviral genome of the adenovirus so as to accommodate the insertion of a non-native nucleic acid sequence, for example, for gene transfer, into the adenovirus.

The adenovirus or adenoviral vector can be replication-competent, conditionally replication-competent, or replication-deficient.

A replication-competent adenovirus or adenoviral vector can replicate in typical host cells, i.e., cells typically capable of being infected by an adenovirus. A replication-competent adenovirus or adenoviral vector can have one or more mutations as compared to the wild-type adenovirus (e.g., one or more deletions, insertions, and/or substitutions) in the adenoviral genome that do not inhibit viral replication in host cells. For example, the adenovirus or adenoviral vector can have a partial or entire deletion of the adenoviral early region known as the E3 region, which is not essential for propagation of the adenovirus or adenoviral genome.

A conditionally-replicating adenovirus or adenoviral vector is an adenovirus or adenoviral vector that has been engineered to replicate under pre-determined conditions. For example, replication-essential gene functions, e.g., gene functions encoded by the adenoviral early regions, can be operably linked to an inducible, repressible, or tissue-specific transcription control sequence, e.g., promoter. In such an embodiment, replication requires the presence or absence of specific factors that interact with the transcription control sequence. Conditionally-replicating adenoviral vectors are further described in U.S. Pat. No. 5,998,205.

A replication-deficient adenovirus or adenoviral vector is an adenovirus or adenoviral vector that requires complementation of one or more gene functions or regions of the adenoviral genome that are required for replication, as a result of, for example, a deficiency in one or more replication-essential gene function or regions, such that the adenovirus or adenoviral vector does not replicate in typical host cells, especially those in a human to be infected by the adenovirus or adenoviral vector.

A deficiency in a gene function or genomic region, as used herein, is defined as a disruption (e.g., deletion) of sufficient genetic material of the adenoviral genome to obliterate or impair the function of the gene (e.g., such that the function of the gene product is reduced by at least about 2-fold, 5-fold, 10-fold, 20-fold, 30-fold, or 50-fold) whose nucleic acid sequence was disrupted (e.g., deleted) in whole or in part. Deletion of an entire gene region often is not required for disruption of a replication-essential gene function. However, for the purpose of providing sufficient space in the adenoviral genome for one or more transgenes, removal of a majority of one or more gene regions may be desirable. While deletion of genetic material is preferred, mutation of genetic material by addition or substitution also is appropriate for disrupting gene function. Replication-essential gene functions are those gene functions that are required for adenovirus replication (e.g., propagation) and are encoded by, for example, the adenoviral early regions (e.g., the E1, E2, and E4 regions), late regions (e.g., the L1, L2, L3, L4, and L5 regions), genes involved in viral packaging (e.g., the IVa2 gene), and virus-associated RNAs (e.g., VA-RNA-1 and/or VA-RNA-2).

Whether the adenovirus or adenoviral vector is replication-competent or replication-deficient, the adenovirus or adenoviral vector retains at least a portion of the adenoviral genome. The adenovirus or adenoviral vector can comprise any portion of the adenoviral genome, including protein coding and non-protein coding regions. Desirably, the adenovirus or adenoviral vector comprises at least one nucleic acid sequence that encodes an adenovirus protein. The adenovirus or adenoviral vector can comprise a nucleic acid sequence that encodes any suitable adenovirus protein, such as, for example, a protein encoded by any one of the early region genes (i.e., E1A, E1B, E2A, E2B, E3, and/or E4 regions), or a protein encoded by any one of the late region genes, which encode the virus structural proteins (i.e., L1, L2, L3, L4, and L5 regions).

The adenovirus or adenoviral vector desirably comprises one or more nucleic acid sequences that encode the pIX protein, the DNA polymerase protein, the penton protein, the hexon protein, and/or the fiber protein. The adenovirus or adenoviral vector can comprise a full-length nucleic acid sequence that encodes a full-length amino acid sequence of an adenovirus protein. Alternatively, the adenovirus or adenoviral vector can comprise a portion of a full-length nucleic acid sequence that encodes a portion of a full-length amino acid sequence of an adenovirus protein.

A “portion” of a nucleic acid sequence comprises at least ten nucleotides (e.g., about 10 to about 5000 nucleotides). Preferably, a “portion” of a nucleic acid sequence comprises 10 or more (e.g., 15 or more, 20 or more, 25 or more, 30 or more, 35 or more, 40 or more, 45 or more, 50 or more, or 100 or more) nucleotides, but less than 5,000 (e.g., 4900 or less, 4000 or less, 3000 or less, 2000 or less, 1000 or less, 800 or less, 500 or less, 300 or less, or 100 or less) nucleotides. Preferably, a portion of a nucleic acid sequence is about 10 to about 3500 nucleotides (e.g., about 10, 20, 30, 50, 100, 300, 500, 700, 1000, 1500, 2000, 2500, or 3000 nucleotides), about 10 to about 1000 nucleotides (e.g., about 25, 55, 125, 325, 525, 725, or 925 nucleotides), or about 10 to about 500 nucleotides (e.g., about 15, 30, 40, 50, 60, 70, 80, 90, 150, 175, 250, 275, 350, 375, 450, 475, 480, 490, 495, or 499 nucleotides), or a range defined by any two of the foregoing values. More preferably, a “portion” of a nucleic acid sequence comprises no more than about 3200 nucleotides (e.g., about 10 to about 3200 nucleotides, about 10 to about 3000 nucleotides, or about 30 to about 500 nucleotides, or a range defined by any two of the foregoing values).

A “portion” of an amino acid sequence comprises at least three amino acids (e.g., about 3 to about 1,200 amino acids). Preferably, a “portion” of an amino acid sequence comprises 3 or more (e.g., 5 or more, 10 or more, 15 or more, 20 or more, 25 or more, 30 or more, 40 or more, or 50 or more) amino acids, but less than 1,200 (e.g., 1,000 or less, 800 or less, 700 or less, 600 or less, 500 or less, 400 or less, 300 or less, 200 or less, or 100 or less) amino acids. Preferably, a portion of an amino acid sequence is about 3 to about 500 amino acids (e.g., about 10, 100, 200, 300, 400, or 500 amino acids), about 3 to about 300 amino acids (e.g., about 20, 50, 75, 95, 150, 175, or 200 amino acids), or about 3 to about 100 amino acids (e.g., about 15, 25, 35, 40, 45, 60, 65, 70, 80, 85, 90, 95, or 99 amino acids), or a range defined by any two of the foregoing values. More preferably, a “portion” of an amino acid sequence comprises no more than about 500 amino acids (e.g., about 3 to about 400 amino acids, about 10 to about 250 amino acids, or about 50 to about 100 amino acids, or a range defined by any two of the foregoing values).

The adenovirus pIX protein is present in the adenovirus capsid, has been shown to strengthen hexon nonamer interactions, and is essential for the packaging of full-length genomes (see, e.g., Boulanger et al., J. Gen. Virol., 44: 783-800 (1979); Horwitz M. S., “Adenoviridae and their replication” in Virology, 2^{nd} ed., B. N. Fields et al. (eds.), Raven Press, Ltd., New York, pp. 1679-1721 (1990), Ghosh-Choudhury et al., EMBO J., 6: 1733-1739 (1987), and van Oostrum et al, J. Virol., 56: 439-448 (1985)). In addition to its contribution to adenovirus structure, pIX also has been shown to exhibit transcriptional properties, such as stimulation of adenovirus major late promoter (MLP) activity (see, e.g., Lutz et al., J. Virol., 71(7): 5102-5109 (1997)). Nucleic acid sequences that encode all or a portion of an adenovirus pIX protein include, for example, SEQ ID NO: 6, SEQ ID NO: 1, SEQ ID NO: 30, SEQ ID NO: 31, SEQ ID NO: 32, SEQ ID NO: 64, and SEQ ID NO: 59. SEQ ID NO: 25 is a subset of SEQ ID NO: 30. Amino acid sequences that comprise a full-length pIX protein, or a portion thereof, include, for example, SEQ ID NO: 16, SEQ ID NO: 11, SEQ ID NO: 41, SEQ ID NO: 42, SEQ ID NO: 74, and SEQ ID NO: 69.

The adenovirus DNA polymerase protein is essential for viral DNA replication both in vitro and in vivo. The polymerase co-purifies in a complex with the precursor (pTP) of the terminal protein (TP), which is covalently attached to the 5′ ends of adenovirus DNA (Field et al., J. Biol. Chem., 259: 9487-9495 (1984)). Both the adenovirus DNA polymerase and pTP are encoded by the E2 region. The polymerase protein is required for the expression of all the structural proteins except for pIX. Without the gene sequence for polymerase protein, polymerase protein is not produced. As a result, the viral genome is not replicated, the Major Late Promoter is not activated, and the capsid proteins are not expressed. Nucleic acid sequences that encode all or a portion of an adenovirus DNA polymerase protein include, for example, SEQ ID NO: 7, SEQ ID NO: 2, SEQ ID NO: 33, and SEQ ID NO: 65. SEQ ID NO: 26 is a subset of SEQ ID NO: 33, and SEQ ID NO: 60 is a subset of SEQ ID NO: 65. Amino acid sequences that comprise a full-length adenovirus DNA polymerase, or a portion thereof, include, for example, SEQ ID NO: 17, SEQ ID NO: 12, SEQ ID NO: 43, SEQ ID NO: 37, SEQ ID NO: 75, and SEQ ID NO: 70.

The adenovirus hexon protein is the largest and most abundant protein in the adenovirus capsid. The hexon protein is essential for virus capsid assembly, determination of the icosahedral symmetry of the capsid (which in turn defines the limits on capsid volume and DNA packaging size), and integrity of the capsid. In addition, hexon is a primary target for modification in order to reduce neutralization of adenoviral vectors (see, e.g., Gall et al., J. Virol., 72: 10260-264 (1998), and Rux et al., J. Virol., 77(17): 9553-9566 (2003)). The major structural features of the hexon protein are shared by adenoviruses across serotypes, but the hexon protein differs in size and immunological properties between serotypes (Jornvall et al., J. Biol. Chem., 256(12): 6181-6186 (1981)). A comparison of 15 adenovirus hexon proteins revealed that the predominant antigenic and serotype-specific regions of the hexon appear to be in loops 1 and 2 (i.e., LI or l1, and LII or l2, respectively), within which are seven discrete hypervariable regions (HVR1 to HVR7) varying in length and sequence between adenoviral serotypes (Crawford-Miksza et al., J. Virol., 70(3): 1836-1844 (1996)). Nucleic acid sequences that encode all or a portion of an adenovirus hexon protein include, for example, SEQ ID NO: 9 and SEQ ID NO: 4, SEQ ID NO: 35, SEQ ID NO: 28, SEQ ID NO: 67, and SEQ ID NO: 62. Amino acid sequences that comprise a full-length adenovirus hexon protein, or a portion thereof, include, for example, SEQ ID NO: 19 and SEQ ID NO: 14, SEQ ID NO: 45, SEQ ID NO: 39, SEQ ID NO: 77, and SEQ ID NO: 72.

The adenovirus fiber protein is a homotrimer of the adenoviral polypeptide IV that has three domains: the tail, shaft, and knob. (Devaux et al., J. Molec. Biol., 215: 567-88 (1990), Yeh et al., Virus Res., 33: 179-98 (1991)). The fiber protein mediates primary viral binding to receptors on the cell surface via the knob and the shaft domains (Henry et al., J. Virol., 68(8): 5239-46 (1994)). The amino acid sequences for trimerization are located in the knob, which appears necessary for the amino terminus of the fiber (the tail) to properly associate with the penton base (Novelli et al., Virology, 185: 365-76 (1991)). In addition to recognizing cell receptors and binding the penton base, the fiber contributes to serotype identity. Fiber proteins from different adenoviral serotypes differ considerably (see, e.g., Green et al., EMBO J., 2: 1357-65 (1983), Chroboczek et al., Virology, 186: 280-85 (1992), and Signas et al., J. Virol., 53: 672-78 (1985)). Thus, the fiber protein has multiple functions key to the life cycle of adenovirus. Nucleic acid sequences that encode all or a portion of an adenovirus fiber protein include, for example, SEQ ID NO: 10 and SEQ ID NO: 5, SEQ ID NO: 36, SEQ ID NO: 29, and SEQ ID NO: 68. SEQ ID NO: 63 is a subset of SEQ ID NO: 68. Amino acid sequences that comprise a full-length adenovirus fiber protein, or a portion thereof, include, for example, SEQ ID NO: 20, SEQ ID NO: 15, SEQ ID NO: 46, SEQ ID NO: 40, SEQ ID NO: 78, and SEQ ID NO: 73.

The adenovirus penton base protein is located at the vertices of the icosahedral capsid and comprises five identical monomers. The penton base protein provides a structure for bridging the hexon proteins on multiple facets of the icosahedral capsid, and provides the essential interface for the fiber protein to be incorporated in the capsid. Each monomer of the penton base contains an RGD tripeptide motif (Neumann et al., Gene, 69: 153-157 (1988)). The RGD tripeptide mediates binding to αv integrins and adenoviruses that have point mutations in the RGD sequence of the penton base are restricted in their ability to infect cells (Bai et al., J. Virol., 67: 5198-5205 (1993)). Thus, the penton base protein is essential for the architecture of the capsid and for maximum efficiency of virus-cell interaction. Nucleic acid sequences that encode all or a portion of an adenovirus penton base protein include, for example, SEQ ID NO: 8 and SEQ ID NO: 3, SEQ ID NO: 34, SEQ ID NO: 27, SEQ ID NO: 66, and SEQ ID NO: 61. Amino acid sequences that comprise a full-length adenovirus penton base protein, or a portion thereof, include, for example, SEQ ID NO: 18, SEQ ID NO: 13, SEQ ID NO: 44, SEQ ID NO: 38, SEQ ID NO: 76, and SEQ ID NO: 71.

Nucleic acid or amino acid sequence “identity,” as described herein, can be determined by comparing a nucleic acid or amino acid sequence of interest to a reference nucleic acid or amino acid sequence. The number of nucleotides or amino acid residues that have been changed and/or modified (such as, e.g., by point mutations, insertions, or deletions) in the reference sequence so as to result in the sequence of interest are counted. The total number of such changes is subtracted from the total length of the sequence of interest, and the difference is divided by the length of the sequence of interest and expressed as a percentage. A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FASTM, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probalistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

In one embodiment, the adenovirus or adenoviral vector comprises one or more of the following nucleic acid sequences: (a) the nucleic acid sequence of SEQ ID NO: 1, (b) a nucleic acid sequence that is at least 98.5% identical (e.g., at least 98.73%, at least 98.96%, at least 99.18%, at least 99.41%, at least 99.64%, at least 99.87%, or 100% identical) to SEQ ID NO: 2, (c) a nucleic acid sequence that is at least 90% identical (e.g., at least 92.94%, at least 95.88%, 98.82%, or 100% identical) to SEQ ID NO: 3, (d) a nucleic acid sequence that is at least 80% identical (e.g., at least 80.83%, at least 83.06%, at least 85.28%, at least 87.50%, at least 89.72%, at least 91.94%, at least 94.17%, at least 96.39%, at least 98.61%, or 100% identical) to SEQ ID NO: 4, and (e) a nucleic acid sequence that is at least 89% identical (e.g., at least 92.33%, at least 95.67%, at least 99%, or 100% identical) to SEQ ID NO: 5.

The adenovirus or adenoviral vector can comprise one, two, three, four, or all five of the aforementioned sequences alone or in any combination. In this respect, the adenovirus or adenoviral vector can comprise any combination of any two of the aforementioned sequences, any combination of any three of the aforementioned sequences, any combination of any four of the aforementioned sequences, or all five of the aforementioned sequences. For example, the adenovirus or adenoviral vector can comprise the nucleic acid sequence of SEQ ID NO: 1. The adenovirus or adenoviral vector can comprise a nucleic acid sequence that is at least 98.5% identical to SEQ ID NO: 2 and a nucleic acid sequence that is at least 90% identical to SEQ ID NO: 3. The adenovirus or adenoviral vector can comprise the nucleic acid sequence of SEQ ID NO: 1, a nucleic acid sequence that is at least 90% identical to SEQ ID NO: 3, and a nucleic acid sequence that is at least 89% identical to SEQ ID NO: 5. The adenovirus or adenoviral vector can comprise (a) the nucleic acid sequence of SEQ ID NO: 1, (b) the nucleic acid sequence SEQ ID NO: 2, (c) the nucleic acid sequence of SEQ ID NO: 3, (d) the nucleic acid sequence of SEQ ID NO: 4, or (e) the nucleic acid sequence of SEQ ID NO: 5. The adenovirus or adenoviral vector can comprise (a) the nucleic acid sequence of SEQ ID NO: 1, (b) a nucleic acid sequence that is at least 98.5% identical to SEQ ID NO: 2, (c) a nucleic acid sequence that is at least 90% identical to SEQ ID NO: 3, (d) a nucleic acid sequence that is at least 80% identical to SEQ ID NO: 4, and (e) a nucleic acid sequence that is at least 89% identical to SEQ ID NO: 5. The adenovirus or adenoviral vector can comprise (a) the nucleic acid sequence of SEQ ID NO: 1, (b) the nucleic acid sequence SEQ ID NO: 2, (c) the nucleic acid sequence of SEQ ID NO: 3, (d) the nucleic acid sequence of SEQ ID NO: 4, and (e) the nucleic acid sequence of SEQ ID NO: 5.

In another embodiment, the adenovirus or adenoviral vector comprises one or more of the following nucleic acid sequences: (a) a nucleic acid sequence that is at least 98.6% identical (e.g., at least 98.85%, at least 99.10%, at least 99.35%, at least 99.60%, or 100% identical) to SEQ ID NO: 6, (b) a nucleic acid sequence that is at least 99.06% identical (e.g., at least 99.09%, at least 99.12%, at least 99.15%, at least 99.19%, at least 99.22%, at least 99.25%, at least 99.28%, at least 99.31%, at least 99.34%, at least 99.38%, at least 99.41%, at least 99.44%, at least 99.47%, at least 99.50%, at least 99.53%, at least 99.57%, at least 99.60%, at least 99.63%, at least 99.66%, at least 99.69%, at least 99.72%, at least 99.75%, at least 99.79%, at least 99.82%, at least 99.85%, at least 99.88%, at least 99.91%, at least 99.94%, at least 99.98%, or 100% identical) to SEQ ID NO: 7, (c) a nucleic acid sequence that is at least 97.13% identical (e.g., at least 97.18%, at least 97.23%, at least 97.28%, at least 97.33%, at least 97.38%, at least 97.43%, at least 97.48%, at least 97.5% at least 97.54%, at least 97.59%, at least 97.6%, at least 97.64%, at least 97.69%, at least 97.7%, at least 97.74%, at least 97.79%, at least 97.8%, at least 97.84%, at least 97.89%, at least 97.9%, at least 97.94%, at least 97.99%, at least 98%, at least 98.04%, at least 98.09%, at least 98.1%, at least 98.14%, at least 98.19%, at least 98.2%, at least 98.24%, at least 98.30%, at least 98.35%, at least 98.40%, at least 98.45%, at least 98.50%, at least 98.55%, at least 98.60%, at least 98.70%, at least 98.75%, at least 98.80%, at least 98.85%, at least 98.90%, at least 98.95%, at least 99.00%, at least 99.06%, at least 99.11%, at least 99.16%, at least 99.2%, at least 99.21%, at least 99.26%, at least 99.3%, at least 99.31%, at least 99.36%, at least 99.4%, at least 99.41%, at least 99.46%, at least 99.5%, at least 99.51%, at least 99.56%, at least 99.6%, at least 99.61%, at least 99.66%, at least 99.7%, at least 99.71%, at least 99.76%, at least 99.8%, at least 99.81%, at least 99.87%, at least 99.9%, at least 99.92%, at least 99.95%, at least 99.97%, or 100% identical) to SEQ ID NO: 8, (d) a nucleic acid sequence that is at least 90.7% identical (e.g., at least 90.73%, at least 90.77%, at least 90.80%, at least 90.84%, at least 90.87%, at least 90.91%, at least 90.94%, at least 90.98%, at least 91.01%, at least 91.05%, at least 91.08%, at least 91.12%, at least 91.15%, at least 91.19%, at least 91.22%, at least 91.26%, at least 91.29%, at least 91.33%, at least 91.36%, at least 91.40%, at least 91.43%, at least 91.46%, at least 91.50%, at least 91.53%, at least 91.57%, at least 91.60%, at least 91.64%, at least 91.67%, at least 91.71%, at least 91.74%, at least 91.78%, at least 91.81%, at least 91.85%, at least 91.88%, at least 91.92%, at least 91.95%, at least 91.99%, at least 92.02%, at least 92.06%, at least 92.09%, at least 92.13%, at least 92.16%, at least 92.19%, at least 92.23%, at least 92.26%, at least 92.30%, at least 92.33%, at least 92.37%, at least 92.40%, at least 92.44%, at least 92.47%, at least 92.51%, at least 92.54%, at least 92.58%, at least 92.61%, at least 92.65%, at least 92.68%, at least 92.72%, at least 92.75%, at least 92.79%, at least 92.82%, at least 92.86%, at least 92.89%, at least 92.92%, at least 92.96%, at least 92.99%, at least 93.03%, at least 93.06%, at least 93.10%, at least 93.13%, at least 93.17%, at least 93.20%, at least 93.24%, at least 93.27%, at least 93.31%, at least 93.34%, at least 93.38%, at least 93.41%, at least 93.45%, at least 93.48%, at least 93.52%, at least 93.55%, at least 93.58%, at least 93.62%, at least 93.65%, at least 93.69%, at least 93.72%, at least 93.76%, at least 93.79%, at least 93.83%, at least 93.86%, at least 93.90%, at least 93.93%, at least 93.97%, at least 94.00%, at least 94.04%, at least 94.07%, at least 94.11%, at least 94.14%, at least 94.18%, at least 94.21%, at least 94.25%, at least 94.28%, at least 94.31%, at least 94.35%, at least 94.38%, at least 94.42%, at least 94.45%, at least 94.49%, at least 94.52%, at least 94.56%, at least 94.59%, at least 94.63%, at least 94.66%, at least 94.70%, at least 94.73%, at least 94.77%, at least 94.80%, at least 94.84%, at least 94.87%, at least 94.91%, at least 94.94%, at least 94.98%, at least 95.01%, at least 95.04%, at least 95.08%, at least 95.11%, at least 95.15%, at least 95.18%, at least 95.22%, at least 95.25%, at least 95.29%, at least 95.32%, at least 95.36%, at least 95.39%, at least 95.43%, at least 95.46%, at least 95.50%, at least 95.53%, at least 95.57%, at least 95.60%, at least 95.64%, at least 95.67%, at least 95.71%, at least 95.74%, at least 95.77%, at least 95.81%, at least 95.84%, at least 95.88%, at least 95.91%, at least 95.95%, at least 95.98%, at least 96.02%, at least 96.05%, at least 96.09%, at least 96.12%, at least 96.16%, at least 96.19%, at least 96.23%, at least 96.26%, at least 96.30%, at least 96.33%, at least 96.37%, at least 96.40%, at least 96.44%, at least 96.47%, at least 96.50%, at least 96.54%, at least 96.57%, at least 96.61%, at least 96.64%, at least 96.68%, at least 96.71%, at least 96.75%, at least 96.78%, at least 96.82%, at least 96.85%, at least 96.89%, at least 96.92%, at least 96.96%, at least 96.99%, at least 97.03%, at least 97.06%, at least 97.10%, at least 97.13%, at least 97.17%, at least 97.20%, at least 97.23%, at least 97.27%, at least 97.30%, at least 97.34%, at least 97.37%, at least 97.41%, at least 97.44%, at least 97.48%, at least 97.51%, at least 97.55%, at least 97.58%, at least 97.62%, at least 97.65%, at least 97.69%, at least 97.72%, at least 97.76%, at least 97.79%, at least 97.83%, at least 97.86%, at least 97.89%, at least 97.93%, at least 97.96%, at least 98.00%, at least 98.03%, at least 98.07%, at least 98.10%, at least 98.14%, at least 98.17%, at least 98.21%, at least 98.24%, at least 98.28%, at least 98.31%, at least 98.35%, at least 98.38%, at least 98.42%, at least 98.45%, at least 98.49%, at least 98.52%, at least 98.56%, at least 98.59%, at least 98.62%, at least 98.66%, at least 98.69%, at least 98.73%, at least 98.76%, at least 98.80%, at least 98.83%, at least 98.87%, at least 98.90%, at least 98.94%, at least 98.97%, at least 99.01%, at least 99.04%, at least 99.08%, at least 99.11%, at least 99.15%, at least 99.18%, at least 99.22%, at least 99.25%, at least 99.29%, at least 99.32%, at least 99.35%, at least 99.39%, at least 99.42%, at least 99.46%, at least 99.49%, at least 99.53%, at least 99.56%, at least 99.60%, at least 99.63%, at least 99.67%, at least 99.70%, at least 99.74%, at least 99.77%, at least 99.81%, at least 99.84%, at least 99.88%, at least 99.91%, at least 99.95%, at least 99.98%, or 100% identical) to SEQ ID NO: 9, and (e) a nucleic acid sequence that is at least 96.6% identical (e.g., at least 96.66%, at least 96.71%, at least 96.77%, at least 96.83%, at least 96.89%, at least 96.94%, at least 97.00%, at least 97.06%, at least 97.11%, at least 97.17%, at least 97.23%, at least 97.29%, at least 97.34%, at least 97.40%, at least 97.46%, at least 97.51%, at least 97.57%, at least 97.63%, at least 97.69%, at least 97.74%, at least 97.80%, at least 97.86%, at least 97.92%, at least 97.97%, at least 98.03%, at least 98.09%, at least 98.14%, at least 98.20%/a, at least 98.26%, at least 98.32%, at least 98.37%, at least 98.43%, at least 98.49%, at least 98.54%, at least 98.60%, at least 98.66%, at least 98.72%, at least 98.77%, at least 98.83%, at least 98.89%, at least 98.94%, at least 99.000/%, at least 99.06%, at least 99.12%, at least 99.17%, at least 99.23%, at least 99.29%, at least 99.34%, at least 99.40%, at least 99.46%, at least 99.52%, at least 99.57%, at least 99.63%, at least 99.69%, at least 99.74%, at least at least 99.80%, at least 99.86%, at least 99.92%, at least 99.97%, or 100% identical) to SEQ ID NO: 10.

The adenovirus or adenoviral vector can comprise one, two, three, four, or all five of the aforementioned sequences alone or in any combination. In this respect, the adenovirus or adenoviral vector can comprise any combination of any two of the aforementioned sequences, any combination of any three of the aforementioned sequences, any combination of any four of the aforementioned sequences, or all five of the aforementioned sequences. For example, the adenovirus or adenoviral vector can comprise a nucleic acid sequence that is at least 98.6% identical to SEQ ID NO: 6. The adenovirus or adenoviral vector can comprise a nucleic acid sequence that is at least 99.06% identical to SEQ ID NO: 7 and a nucleic acid sequence that is at least 97.13% identical to SEQ ID NO: 8. The adenovirus or adenoviral vector can comprise a nucleic acid sequence that is at least 97.13% identical to SEQ ID NO: 8, a nucleic acid sequence that is at least 90.7% identical to SEQ ID NO: 9, and a nucleic acid sequence that is at least 96.6% identical to SEQ ID NO: 10. The adenovirus or adenoviral vector can comprise (a) the nucleic acid sequence of SEQ ID NO: 6, (b) the nucleic acid sequence SEQ ID NO: 7, (c) the nucleic acid sequence of SEQ ID NO: 8, (d) the nucleic acid sequence of SEQ ID NO: 9, or (e) the nucleic acid sequence of SEQ ID NO: 10. The adenovirus or adenoviral vector can comprise (a) a nucleic acid sequence that is at least 98.6% identical to SEQ ID NO: 6, (b) a nucleic acid sequence that is at least 99.06% identical to SEQ ID NO: 7, (c) a nucleic acid sequence that is at least 97.13% identical to SEQ ID NO: 8, (d) a nucleic acid sequence that is at least 90.7% identical to SEQ ID NO: 9, and (e) a nucleic acid sequence that is at least 96.6% identical to SEQ ID NO: 10. The adenovirus or adenoviral vector can comprise (a) the nucleic acid sequence of SEQ ID NO: 6, (b) the nucleic acid sequence SEQ ID NO: 7, (c) the nucleic acid sequence of SEQ ID NO: 8, (d) the nucleic acid sequence of SEQ ID NO: 9, and (e) the nucleic acid sequence of SEQ ID NO: 10.

In another embodiment, the adenovirus or adenoviral vector comprises one or more of the following nucleic acid sequences: (a) a nucleic acid sequence comprising at least 121 contiguous nucleotides of SEQ ID NO: 6, (b) a nucleic acid sequence comprising at least 462 contiguous nucleotides of SEQ ID NO: 7, (c) a nucleic acid sequence comprising at least 234 contiguous nucleotides of SEQ ID NO: 8, (d) a nucleic acid sequence comprising at least 606 contiguous nucleotides of SEQ ID NO: 9, or (e) a nucleic acid sequence comprising at least 188 contiguous nucleotides of SEQ ID NO: 10.

The adenovirus or adenoviral vector can comprise a nucleic acid sequence comprising at least 121 (e.g., 125 or more, 130 or more, 150 or more, 200 or more, 250 or more, or 300 or more) contiguous nucleotides of SEQ ID NO: 6, but no more than 399 (e.g., 398 or less, 350 or less, or 275 or less) contiguous nucleotides of SEQ ID NO: 6. Preferably, the adenovirus or adenoviral vector comprises a nucleic acid sequence comprising 121 to 300 contiguous nucleotides (e.g., 125, 150, 175, 200, 250, or 275 contiguous nucleotides), 121 to 200 contiguous nucleotides (e.g., 130, 140, 145, 160, 165, 170, 180, 185, 190, 195, or 199 contiguous nucleotides), or 121 to 150 contiguous nucleotides (e.g., 122, 123, 124, 125, 126, 127, 128, 129, 130, 131, 132, 133, 134, 135, 136, 137, 138, 139, 140, 141, 142, 143, 144, 145, 146, 147, 148, or 149 contiguous nucleotides) of SEQ ID NO: 6, or a range defined by any two of the foregoing values.

The adenovirus or adenoviral vector can comprise a nucleic acid sequence comprising at least 462 (e.g., 470 or more, 500 or more, 600 or more, 700 or more, 800 or more, 900 or more, or 1,000 or more) contiguous nucleotides of SEQ ID NO: 7, but no more than 3168 (e.g., 3,100 or less, 3,000 or less, 2,500 or less, 2,000 or less, or 1,500 or less) contiguous nucleotides of SEQ ID NO: 7. Preferably, the adenovirus or adenoviral vector comprises a nucleic acid sequence comprising 462 to 2,000 contiguous nucleotides (e.g., 475, 500, 700, 1,000, 1,200, 1,500, or 1,700 contiguous nucleotides), 462 to 1,000 contiguous nucleotides (e.g., 490, 525, 575, 600, 650, 675, 725, 750, 800, 850, 900, or 950 contiguous nucleotides), or 462 to 800 contiguous nucleotides (e.g., 480, 485, 490, 495, 499, 510, 515, 530, 540, 550, 560, 565, 570, 580, 585, 590, 595, 615, 625, 630, 640, 660, 665, 670, 680, 685, 690, 695, 705, 715, 730, 740, 755, 760, 765, 770, 775, 780, 785, 790, 795, or 799 contiguous nucleotides) of SEQ ID NO: 7, or a range defined by any two of the foregoing values.

The adenovirus or adenoviral vector can comprise a nucleic acid sequence comprising at least 234 (e.g., 235 or more, 250 or more, 300 or more, 350 or more, 400 or more, 450 or more, or 500 or more) contiguous nucleotides of SEQ ID NO: 8, but no more than 1,974 (e.g., 1,900 or less, 1,800 or less, 1,500 or less, 1,200 or less, 1,000 or less, 850 or less, 800 or less, 750 or less, or 700 or less) contiguous nucleotides of SEQ ID NO: 8. Preferably, the adenovirus or adenoviral vector comprises a nucleic acid sequence comprising 234 to 1,500 contiguous nucleotides (e.g., 290, 300, 400, 500, 600, 700, 800, 900, 1,000, or 1,200 contiguous nucleotides), 234 to 1,000 contiguous nucleotides (e.g., 295, 350, 450, 550, 650, 750, 850, or 950 contiguous nucleotides), or 234 to 500 contiguous nucleotides (e.g., 290, 305, 310, 315, 325, 340, 345, 360, 365, 370, 375, 380, 385, 390, 395, 405, 425, 430, 440, 455, 460, 465, 470, 475, 480, 485, 490, 495, or 499 contiguous nucleotides) of SEQ ID NO: 8, or a range defined by any two of the foregoing values.

The adenovirus or adenoviral vector can comprise a nucleic acid sequence comprising at least 606 (e.g., 610 or more, 650 or more, 700 or more, 800 or more, or 1,000 or more) contiguous nucleotides of SEQ ID NO: 9, but no more than 2877 (2,800 or less, 2,500 or less, 2,000 or less, 1,800 or less, or 1,500 or less) contiguous nucleotides of SEQ ID NO: 9.

Preferably, the adenovirus or adenoviral vector comprises a nucleic acid sequence comprising 606 to 2,000 contiguous nucleotides (e.g., 615, 650, 700, 800, 900, 1,000, 1,200, 1,500, 1,700, or 1,900 contiguous nucleotides), 606 to 1,000 contiguous nucleotides (e.g., 630, 645, 665, 675, 725, 750, 775, 825, 850, 875, 925, 950, or 975 contiguous nucleotides), or 606 to 800 contiguous nucleotides (e.g., 620, 635, 640, 655, 660, 670, 680, 685, 690, 695, 699, 705, 715, 730, 735, 740, 745, 755, 760, 765, 770, 785, 790, 795, or 799 contiguous nucleotides) of SEQ ID NO: 9, or a range defined by any two of the foregoing values.

The adenovirus or adenoviral vector can comprise a nucleic acid sequence comprising at least 188 (e.g., 189 or more, 200 or more, 300 or more, 500 or more, 700 or more, or 900 or more) contiguous nucleotides of SEQ ID NO: 10, but no more than 1,749 (1,700 or less, 1,500 or less, 1,200 or less, or 1,000 or less) contiguous nucleotides of SEQ ID NO: 10. Preferably, the adenovirus or adenoviral vector comprises a nucleic acid sequence comprising 188 to 1,500 contiguous nucleotides (e.g., 200, 400, 600, 800, 1,000, 1,200, or 1,400 contiguous nucleotides), 188 to 1,000 contiguous nucleotides (e.g., 195, 250, 350, 450, 550, 650, 750, 850, or 950 contiguous nucleotides), or 188 to 500 contiguous nucleotides (e.g., 190, 225, 230, 240, 255, 260, 265, 270, 275, 315, 325, 330, 340, 355, 360, 365, 370, 375, 380, 385, 390, 395, 415, 425, 430, 440, 455, 460, 465, 470, 475, 480, 485, 490, 495, or 499 contiguous nucleotides) of SEQ ID NO: 10, or a range defined by any two of the foregoing values.

The adenovirus or adenoviral vector can comprise one, two, three, four, or all five of the aforementioned sequences alone, or in any combination. In this respect, the adenovirus or adenoviral vector can comprise any combination of any two of the aforementioned sequences, any combination of any three of the aforementioned sequences, any combination of any four of the aforementioned sequences, or all five of the aforementioned sequences. For example, the adenovirus or adenoviral vector can comprise a nucleic acid sequence comprising at least 121 contiguous nucleotides of SEQ ID NO: 6. The adenovirus or adenoviral vector can comprise a nucleic acid sequence comprising at least 234 contiguous nucleotides of SEQ ID NO: 8, and a nucleic acid sequence comprising at least 188 contiguous nucleotides of SEQ ID NO: 10. The adenovirus or adenoviral vector can comprise a nucleic acid sequence comprising at least 462 contiguous nucleotides of SEQ ID NO: 7, a nucleic acid sequence comprising at least 606 contiguous nucleotides of SEQ ID NO: 9, and a nucleic acid sequence comprising at least 188 contiguous nucleotides of SEQ ID NO: 10. The adenovirus or adenoviral vector can comprise a nucleic acid sequence comprising at least 121 contiguous nucleotides of SEQ ID NO: 6, a nucleic acid sequence comprising at least 462 contiguous nucleotides of SEQ ID NO: 7, a nucleic acid sequence comprising at least 234 contiguous nucleotides of SEQ ID NO: 8, and a nucleic acid sequence comprising at least 606 contiguous nucleotides of SEQ ID NO: 9. The adenovirus or adenoviral vector can comprise (a) a nucleic acid sequence comprising at least 121 contiguous nucleotides of SEQ ID NO: 6, (b) a nucleic acid sequence comprising at least 462 contiguous nucleotides of SEQ ID NO: 7, (c) a nucleic acid sequence comprising at least 234 contiguous nucleotides of SEQ ID NO: 8, (d) a nucleic acid sequence comprising at least 606 contiguous nucleotides of SEQ ID NO: 9, and (e) a nucleic acid sequence comprising at least 188 contiguous nucleotides of SEQ ID NO: 10.

In another embodiment, the adenovirus or adenoviral vector comprises one or more of the following amino acid sequences: (a) the amino acid sequence of SEQ ID NO: 11, (b) an amino acid sequence that is at least 82% identical (e.g., at least 88.67%, at least 95.33%, or 100% identical) to SEQ ID NO: 13, (c) an amino acid sequence that is at least 80% identical (e.g., at least 81%, at least 82%, at least 83%, at least 83.06%, at least 84%, at least 85%, at least 85.28%, at least 86%, at least 87%, at least 87.5%, at least 88%, at least 88.67%, at least 89%, at least 89.72% at least 90%, at least 91%, at least 91.94%, at least 92%, at least 92.33%, at least 93%, at least 94%, at least 94.17%, at least 95%, at least 95.33%, at least 95.67%, at least 96%, at least 96.39%, at least 97%, at least 98%, at least 98.61%, at least 99%, at least 99.5%, or 100% identical) to SEQ ID NO: 14, and (d) an amino acid sequence that is at least 83% identical (e.g., at least 89.67%, at least 96.33%, or 100% identical) to SEQ ID NO: 15.

The adenovirus or adenoviral vector can comprise one, two, three, or all four of the aforementioned amino acid sequences alone or in any combination. In this respect, the adenovirus or adenoviral vector can comprise any combination of any two of the aforementioned sequences, any combination of any three of the aforementioned sequences, or all four of the aforementioned sequences. For example, the adenovirus or adenoviral vector can comprise the amino acid sequence of SEQ ID NO: 11. The adenovirus or adenoviral vector can comprise an amino acid sequence of SEQ ID NO: 11, and an amino acid sequence that is at least 82% identical to SEQ ID NO: 13. The adenovirus or adenoviral vector can comprise the amino acid sequence of SEQ ID NO: 11, an amino acid sequence that is at least 82% identical to SEQ ID NO: 13, and an amino acid sequence that is at least 83% identical to SEQ ID NO: 15. The adenovirus or adenoviral vector can comprise (a) the amino acid sequence of SEQ ID NO: 11, (b) the amino acid sequence of SEQ ID NO: 13, (c) the amino acid sequence of SEQ ID NO: 14, or (d) the amino acid sequence of SEQ ID NO: 15. The adenovirus or adenoviral vector can comprise (a) the amino acid sequence of SEQ ID NO: 11, (b) an amino acid sequence that is at least 82% identical to SEQ ID NO: 13, (c) an amino acid sequence that is at least 80% identical to SEQ ID NO: 14, and (d) an amino acid sequence that is at least 83% identical to SEQ ID NO: 15. The adenovirus or adenoviral vector can comprise (a) the amino acid sequence of SEQ ID NO: 11, (b) the amino acid sequence of SEQ ID NO: 13, (c) the amino acid sequence of SEQ ID NO: 14, and (d) the amino acid sequence of SEQ ID NO: 15.

In another embodiment, the adenovirus or adenoviral vector comprises one or more of the following amino acid sequences: (a) the amino acid sequence of SEQ ID NO: 16, (b) an amino acid sequence that is at least 97.8% identical (e.g., at least 97.95%, at least 98.10%, at least 98.26%, at least 98.41%, at least 98.56%, at least 98.710%, at least 98.86%, at least 99.02%, at least 99.17%, at least 99.32%, at least 99.47%, at least 99.62%, at least 99.78%, or 100% identical) to SEQ ID NO: 18, (c) an amino acid sequence that is at least 93.4% identical (e.g., at least 93.50%, at least 93.61%, at least 93.71%, at least 93.82%, at least 93.92%, at least 94.03%, at least 94.13%, at least 94.23%, at least 94.34%, at least 94.44%, at least 94.55%, at least 94.65%, at least 94.76%, at least 94.86%, at least 94.96%, at least 95.07%, at least 95.17%, at least 95.28%, at least 95.38%, at least 95.49%, at least 95.59%, at least 95.69%, at least 95.80%, at least 95.90%, at least 96.01%, at least 96.11%, at least 96.22%, at least 96.32%, at least 96.42%, at least 96.53%, at least 96.63%, at least 96.74%, at least 96.84%, at least 96.95%, at least 97.05%, at least 97.15%, at least 97.26%, at least 97.36%, at least 97.47%, at least 97.57%, at least 97.68%, at least 97.78%, at least 97.88%, at least 97.99%, at least 98.09%, at least 98.20%, at least 98.30%, at least 98.41%, at least 98.51%, at least 98.61%, at least 98.72%, at least 98.82%, at least 98.93%, at least 99.03%, at least 99.14%, at least 99.24%, at least 99.34%, at least 99.45%, at least 99.55%, at least 99.66%, at least 99.76%, at least 99.87%, at least 99.97%, or 100% identical) to SEQ ID NO: 19, and (d) an amino acid sequence that is at least 98.2% identical (e.g., at least 98.37%, at least 98.54%, at least 98.71%, at least 98.89%, at least 99.06%, at least 99.23%, at least 99.40%, at least 99.57%, at least 99.74%, at least 99.92%, or 100% identical) to SEQ ID NO: 20.

The adenovirus or adenoviral vector can comprise one, two, three, or all four of the aforementioned amino acid sequences alone or in any combination. In this respect, the adenovirus or adenoviral vector can comprise any combination of any two of the aforementioned sequences, any combination of any three of the aforementioned sequences, or all four of the aforementioned sequences. For example, the adenovirus or adenoviral vector can comprise the amino acid sequence of SEQ ID NO: 16. The adenovirus or adenoviral vector can comprise the amino acid sequence of SEQ ID NO: 16, and an amino acid sequence that is at least 93.4% identical to SEQ ID NO: 19. The adenovirus or adenoviral vector can comprise the amino acid sequence of SEQ ID NO: 16, an amino acid sequence that is at least 93.4% identical to SEQ ID NO: 19, and an amino acid sequence that is at least 98.2% identical to SEQ ID NO: 20. The adenovirus or adenoviral vector can comprise (a) the amino acid sequence of SEQ ID NO: 16, (b) the amino acid sequence of SEQ ID NO: 18, (c) the amino acid sequence of SEQ ID NO: 19, or (d) the amino acid sequence of SEQ ID NO: 20. The adenovirus or adenoviral vector can comprise (a) the amino acid sequence of SEQ ID NO: 16, (b) an amino acid sequence that is at least 97.8% identical to SEQ ID NO: 18, (c) an amino acid that is at least 93.4% identical to SEQ ID NO: 19, and (d) an amino acid sequence that is at least 98.2% identical to SEQ ID NO: 20.

The adenovirus or adenoviral vector can comprise (a) the amino acid sequence of SEQ ID NO: 16, (b) the amino acid sequence of SEQ ID NO: 18, (c) the amino acid sequence of SEQ ID NO: 19, and (d) the amino acid sequence of SEQ ID NO: 20.

In another embodiment, the adenovirus or adenoviral vector comprises one or more of the following amino acid sequences: (a) an amino acid sequence comprising at least 89 contiguous amino acid residues of SEQ ID NO: 16, (b) an amino acid sequence comprising at least 247 contiguous amino acid residues of SEQ ID NO: 18, (c) an amino acid sequence comprising at least 370 contiguous amino acid residues of SEQ ID NO: 19, and (d) an amino acid sequence comprising at least 192 contiguous amino acid residues of SEQ ID NO: 20.

The adenovirus or adenoviral vector can comprise an amino acid sequence comprising at least 89 (e.g., 90 or more, 100 or more, or 110 or more) contiguous amino acid residues of SEQ ID NO: 16, but no more than 133 (e.g., 130 or less, 125 or less, 120 or less, or 115 or less) contiguous amino acid residues of SEQ ID NO: 16. Preferably, the adenovirus or adenoviral vector comprises an amino acid sequence comprising 89 to 130 contiguous amino acid residues (e.g., 90, 100, 110, 115, 120, or 125 contiguous amino acid residues) of SEQ ID NO: 16, 89 to 115 contiguous amino acid residues of SEQ ID NO: 16 (e.g., 95, 110, or 112 contiguous amino acid residues), or 89 to 100 contiguous amino acid residues (e.g., 89, 90, 91, 92, 93, 94, 95, 96, 97, 98, or 99 contiguous amino acid residues) of SEQ ID NO: 16, or a range defined by any two of the foregoing values.

The adenovirus or adenoviral vector can comprise an amino acid sequence comprising at least 247 (e.g., 250 or more, 275 or more, 300 or more, or 400 or more) contiguous amino acid residues of SEQ ID NO: 18, but no more than 658 (e.g., 650 or less, 550 or less, or 450 or less) contiguous amino acid residues of SEQ ID NO: 18. Preferably, the adenovirus or adenoviral vector comprises an acid sequence comprising 247 to 600 contiguous amino acid residues (e.g., 255, 275, 300, 400, or 500 contiguous amino acid residues) of SEQ ID NO: 18, 247 to 500 contiguous amino acid residues of SEQ ID NO: 18 (e.g., 325, 350, 375, 425, 450, or 475 contiguous amino acid residues), or 247 to 400 contiguous amino acid residues (e.g., 265, 280, 285, 290, 295, 360, 365, 380, 385, 390, 395, or 399 contiguous amino acid residues) of SEQ ID NO: 18, or a range defined by any two of the foregoing values.

The adenovirus or adenoviral vector can comprise an amino acid sequence comprising at least 370 (e.g., 380 or more, 400 or more, or 500 or more) contiguous amino acid residues of SEQ ID NO: 19, but no more than 959 (e.g., 950 or less, 900 or less, 800 or less, 700 or less, or 600 or less) contiguous amino acid residues of SEQ ID NO: 19. Preferably, the adenovirus or adenoviral vector comprises an acid sequence comprising 370 to 800 contiguous amino acid residues (e.g., 390, 400, 500, 600, or 700 contiguous amino acid residues) of SEQ ID NO: 19, 370 to 600 contiguous amino acid residues (e.g., 375, 385, 395, 425, 445, 450, 465, 475, 525, 545, 550, 565 or 575 contiguous amino acid residues) of SEQ ID NO: 19, or 370 to 500 contiguous amino acid residues (e.g., 385, 389, 395, 399, 415, 435, 440, 460, 470, 480, or 499 contiguous amino acid residues) of SEQ ID NO: 19, or a range defined by any two of the foregoing values.

The adenovirus or adenoviral vector can comprise an amino acid sequence comprising at least 192 (e.g., 193 or more, 200 or more, or 300 or more) contiguous amino acid residues of SEQ ID NO: 20, but no more than 583 (e.g., 580 or less, 550 or less, 500 or less, 450 or less, or 400 or less) contiguous amino acid residues of SEQ ID NO: 20. Preferably, the adenovirus or adenoviral vector comprises an acid sequence comprising 192 to 500 contiguous amino acid residues (e.g., 198, 200, 300, or 400 contiguous amino acid residues) of SEQ ID NO: 20, 192 to 300 contiguous amino acid residues (e.g., 194, 196, 200, 210, 220, 230, 240, 250, 260, 270, 280, or 290 contiguous amino acid residues) of SEQ ID NO: 20, or 192 to 250 contiguous amino acid residues (e.g., 195, 199, 215, 225, 235, or 245 contiguous amino acid residues) of SEQ ID NO: 20, or a range defined by any two of the foregoing values.

The adenovirus or adenoviral vector can comprise one, two, three, or all four of the aforementioned amino acid sequences alone or in any combination. In this respect, the adenovirus or adenoviral vector can comprise any combination of any two of the aforementioned sequences, any combination of any three of the aforementioned sequences, or all four of the aforementioned sequences. For example, the adenovirus or adenoviral vector can comprise an amino acid sequence comprising at least 89 contiguous amino acid residues of SEQ ID NO: 16. The adenovirus or adenoviral vector can comprise an amino acid sequence comprising at least 89 contiguous amino acid residues of SEQ ID NO: 16, and an amino acid sequence comprising at least 370 contiguous amino acid residues of SEQ ID NO: 19. The adenovirus or adenoviral vector can comprise an amino acid sequence comprising at least 89 contiguous amino acid residues of SEQ ID NO: 16, an amino acid sequence comprising at least 247 contiguous amino acid residues of SEQ ID NO: 18, and an amino acid sequence comprising at least 370 contiguous amino acid residues of SEQ ID NO: 19. The adenovirus or adenoviral vector can comprise an amino acid sequence comprising at least 89 contiguous amino acid residues of SEQ ID NO: 16, an amino acid sequence comprising at least 247 contiguous amino acid residues of SEQ ID NO: 18, and an amino acid sequence comprising at least 192 contiguous amino acid residues of SEQ ID NO: 20. The adenovirus or adenoviral vector can comprise (a) an amino acid sequence comprising at least 89 contiguous amino acid residues of SEQ ID NO: 16, (b) an amino acid sequence comprising at least 247 contiguous amino acid residues of SEQ ID NO: 18, (c) an amino acid sequence comprising at least 370 contiguous amino acid residues of SEQ ID NO: 19, and (d) an amino acid sequence comprising at least 192 contiguous amino acid residues of SEQ ID NO: 20.

In other embodiments, the adenovirus or adenoviral vector comprises one or more nucleic acid sequences that encode one or more of any of the aforementioned amino acid sequences, e.g., the amino acid sequences of any of SEQ ID NOs: 11-20 or any of the variants and/or portions thereof as described herein. For example, the adenovirus or adenoviral vector can comprise a nucleic acid sequence encoding an amino acid sequence that is at least 99.78% identical (e.g., at least 99.87%, at least 99.97%, or 100% identical) to SEQ ID NO: 17, or a nucleic acid sequence encoding an amino acid sequence that is at least 99% identical (e.g., at least 99.68% or 100% identical) to SEQ ID NO: 12.

In one embodiment, the adenovirus or adenoviral vector comprises one or more of the following nucleic acid sequences: (a) a nucleic acid sequence that is at least 96% identical (e.g., at least 96% or 100% identical) to SEQ ID NO: 25, (b) a nucleic acid sequence that is at least 99% identical (e.g., at least 99.08%, at least 99.16%, at least 99.23%, at least 99.31%, at least 99.39%, at least 99.47%, at least 99.55%, at least 99.62%, at least 99.70%, at least 99.78%, at least 99.86%, at least 99.93%, or 100% identical) to SEQ ID NO: 26, (c) a nucleic acid sequence that is at least 80% identical (e.g., at least 82%, at least 84.22%, at least 86.44%, at least 88.67%, at least 90.89%, at least 93.11%, at least 95.33%, at least 97.56%, at least 99.78%, or 100% identical) to SEQ ID NO: 27, (d) a nucleic acid sequence that is at least 80% identical (e.g., at least 80.58%, at least 82.67%, at least 84.75%, at least 86.83%, at least 88.92%, at least 91.00%, at least 93.08%, at least 95.17%, at least 97.25%, at least 99.33%, or 100% identical) to SEQ ID NO: 28, and (e) a nucleic acid sequence that is at least 85.4% identical (e.g., at least 87.48%, at least 89.57%, at least 91.65%, at least 93.73%, at least 95.82%, at least 97.90%, at least 99.98%, or 100% identical) to SEQ ID NO: 29.

The adenovirus or adenoviral vector can comprise one, two, three, four, or all five of the aforementioned sequences alone or in any combination. In this respect, the adenovirus or adenoviral vector can comprise any combination of any two of the aforementioned sequences, any combination of any three of the aforementioned sequences, any combination of any four of the aforementioned sequences, or all five of the aforementioned sequences. For example, the adenovirus or adenoviral vector can comprise a nucleic acid sequence that is at least 96% identical to SEQ ID NO: 25. The adenovirus or adenoviral vector can comprise a nucleic acid sequence that is at least 99% identical to SEQ ID NO: 26 and a nucleic acid sequence that is at least 80% identical to SEQ ID NO: 27. The adenovirus or adenoviral vector can comprise a nucleic acid sequence that is at least 96% identical to SEQ ID NO: 25, a nucleic acid sequence that is at least 80% identical to SEQ ID NO: 27, and a nucleic acid sequence that is at least 85.4% identical to SEQ ID NO: 29. The adenovirus or adenoviral vector can comprise (a) the nucleic acid sequence of SEQ ID NO: 25, (b) the nucleic acid sequence SEQ ID NO: 26, (c) the nucleic acid sequence of SEQ ID NO: 27, (d) the nucleic acid sequence of SEQ ID NO: 28, or (e) the nucleic acid sequence of SEQ ID NO: 29. The adenovirus or adenoviral vector can comprise (a) a nucleic acid sequence that is at least 96% identical to SEQ ID NO: 25, (b) a nucleic acid sequence that is at least 99% identical to SEQ ID NO: 26, (c) a nucleic acid sequence that is at least 80% identical to SEQ ID NO: 27, (d) a nucleic acid sequence that is at least 80% identical to SEQ ID NO: 28, and (e) a nucleic acid sequence that is at least 85.4% identical to SEQ ID NO: 29. The adenovirus or adenoviral vector can comprise (a) the nucleic acid sequence of SEQ ID NO: 25, (b) the nucleic acid sequence SEQ ID NO: 26, (c) the nucleic acid sequence of SEQ ID NO: 27, (d) the nucleic acid sequence of SEQ ID NO: 28, and (e) the nucleic acid sequence of SEQ ID NO: 29.

In another embodiment, the adenovirus or adenoviral vector comprises one or more of the following nucleic acid sequences: (a) a nucleic acid sequence that is at least 98.7% identical (e.g., at least 98.95%, at least 99.20%, at least 99.45%, at least 99.70%, at least 99.95%, or 100% identical) to SEQ ID NO: 30, (b) a nucleic acid sequence that is at least 98.9% identical (e.g., at least 99.15%, at least 99.40%, at least 99.65%, at least 99.90%, or 100% identical) to SEQ ID NO: 31, (c) a nucleic acid sequence that is at least 99.4% identical (e.g., at least 99.65%, at least 99.90%, or 100% identical) to SEQ ID NO: 32, (d) a nucleic acid sequence that is at least 99.1% identical (e.g., at least 99.13%, at least 99.16%, at least 99.19%, at least 99.23%, at least 99.26%, at least 99.29%, at least 99.32%, at least 99.35%, at least 99.38%, at least 99.42%, at least 99.45%, at least 99.48%, at least 99.51%, at least 99.54%, at least 99.57%, at least 99.61%, at least 99.64%, at least 99.67%, at least 99.70%, at least 99.73%, at least 99.76%, at least 99.79%, at least 99.83%, at least 99.86%, at least 99.89%, at least 99.92%, at least 99.95%, at least 99.98%, or 100% identical) to SEQ ID NO: 33, (e) a nucleic acid sequence that is at least 81.25% identical (e.g., at least 81.30%, at least 81.36%, at least 81.41%, at least 81.46%, at least 81.52%, at least 81.57%, at least 81.63%, at least 81.68%, at least 81.73%, at least 81.79%, at least 81.84%, at least 81.89%, at least 81.95%, at least 82.00%, at least 82.06%, at least 82.11%, at least 82.16%, at least 82.22%, at least 82.27%, at least 82.32%, at least 82.38%, at least 82.43%, at least 82.48%, at least 82.54%, at least 82.59%, at least 82.65%, at least 82.70%, at least 82.75%, at least 82.81%, at least 82.86%, at least 82.91%, at least 82.97%, at least 83.02, at least 83.08%, at least 83.13%, at least 83.18%, at least 83.24%, at least 83.29%, at least 83.34%, at least 83.40%, at least 83.45%, at least 83.50%, at least 83.56%, at least 83.61%, at least 83.67%, at least 83.72%, at least 83.77%, at least 83.83%, at least 83.88%, at least 83.93%, at least 83.99%, at least 84.04%, at least 84.09%, at least 84.15%, at least 84.20%, at least 84.26%, at least 84.31%, at least 84.36%, at least 84.42%, at least 84.47%, at least 84.52%, at least 84.58%, at least 84.63%, at least 84.69%, at least 84.74%, at least 84.79%, at least 84.85%, at least 84.90%, at least 84.95%, at least 85.01%, at least 85.06%, at least 85.11%, at least 85.17%, at least 85.22%, at least 85.28%, at least 85.33%, at least 85.38%, at least 85.44%, at least 85.49%, at least 85.54%, at least 85.60%, at least 85.65%, at least 85.71%, at least 85.76%, at least 85.81%, at least 85.87%, at least 85.92%, at least 85.97%, at least 86.03%, at least 86.08%, at least 86.13%, at least 86.19%, at least 86.24%, at least 86.30%, at least 86.35%, at least 86.40%, at least 86.46%, at least 86.51%, at least 86.56%, at least 86.62%, at least 86.67%, at least 86.73%, at least 86.78%, at least 86.83%, at least 86.89%, at least 86.94%, at least 86.99%, at least 87.05%, at least 87.10%, at least 87.15%, at least 87.21%, at least 87.26%, at least 87.32%, at least 87.37%, at least 87.42%, at least 87.48%, at least 87.53%, at least 87.58%, at least 87.64%, at least 87.69%, at least 87.74%, at least 87.80%, at least 87.85%, at least 87.91%, at least 87.96%, at least 88.01%, at least 88.07%, at least 88.12%, at least 88.17%, at least 88.23%, at least 88.28%, at least 88.34%, at least 88.39%, at least 88.44%, at least 88.50%, at least 88.55%, at least 88.60%, at least 88.66%, at least 88.71%, at least 88.76%, at least 88.82%, at least 88.87%, at least 88.93%, at least at least 88.98%, at least 89.03%, at least 89.09%, at least 89.14%, at least 89.19%, at least 89.25%, at least 89.30%, 89.36%, at least 89.41%, at least 89.46%, at least 89.52%, at least 89.57%, at least 89.62%, at least 89.68%, at least 89.73%, at least 89.78%, at least 89.84%, at least 89.89%, at least 89.95%, at least 90.00%, at least 90.05%, at least 90.11%, at least 90.16%, at least 90.21%, at least 90.27%, at least 90.32%, at least 90.38%, at least 90.43%, at least 90.48%, at least 90.54%, at least 90.59%, at least 90.64%, at least 90.70%, at least 90.75%, at least 90.80%, at least 90.86%, at least 90.91%, at least 90.97%, at least 91.02%, at least 91.07%, at least 91.13%, at least 91.18%, at least 91.23%, at least 91.29%, at least 91.34%, at least 91.39%, at least 91.45%, at least 91.50%, at least 91.56%, at least 91.61%, at least 91.66%, at least 91.72%, at least 91.77%, at least 91.82%, at least 91.88%, at least 91.93%, at least 91.99%, at least 92.04%, at least 92.09%, at least 92.15%, at least 92.20%, at least 92.25%, at least 92.31%, at least 92.36%, at least 92.41%, at least 92.47%, at least 92.52%, at least 92.58%, at least 92.63%, at least 92.68%, at least 92.74%, at least 92.79%, at least 92.84%, at least 92.90%, at least 92.95%, at least 93.01%, at least 93.06%, at least 93.11%, at least 93.17%, at least 93.22%, at least 93.27%, at least 93.33%, at least 93.38%, at least 93.43%, at least 93.49%, at least 93.54%, at least 93.60%, at least 93.65%, at least 93.70%, at least 93.76%, at least 93.81%, at least 93.86%, at least 93.92%, at least 93.97%, at least 94.03%, at least 94.08%, at least 94.13%, at least 94.19%, at least 94.24%, at least 94.29%, at least 94.35%, at least 94.40%, at least 94.45%, at least 94.51%, at least 94.56%, at least 94.62%, at least 94.67%, at least 94.72%, at least 94.78%, at least 94.83%, at least 94.88%, at least 94.94%, at least 94.99%, at least 95.04%, at least 95.10%, at least 95.15%, at least 95.21%, at least 95.26%, at least 95.31%, at least 95.37%, at least 95.42%, at least 95.47%, at least 95.53%, at least 95.58%, at least 95.64%, at least 95.69%, at least 95.74%, at least 95.80%, at least 95.85%, at least 95.90%, at least 95.96%, at least 96.01%, at least 96.06%, at least 96.12%, at least 96.17%, at least 96.23%, at least 96.28%, at least 96.33%, at least 96.39%, at least 96.44%, at least 96.49%, at least 96.55%, at least 96.60%, at least 96.66%, at least 96.71%, at least 96.76%, at least 96.82%, at least 96.87%, at least 96.92%, at least 96.98%, at least 97.03%, at least 97.08%, at least 97.14%, at least 97.19%, at least 97.25%, at least 97.30%, at least 97.35%, at least 97.41%, at least 97.46%, at least 97.51%, at least 97.57%, at least 97.62%, at least 97.68%, at least 97.73%, at least 97.78%, at least 97.84%, at least 97.89%, at least 97.94%, at least 98.00%, at least 98.05%, at least 98.10%, at least 98.16%, at least 98.21%, at least 98.27%, at least 98.32%, at least 98.37%, at least 98.43%, at least 98.48%, at least 98.53%, at least 98.59%, at least 98.64%, at least 98.69%, at least 98.75%, at least 98.80%, at least 98.86%, at least 98.91%, at least 98.96%, at least 99.02%, at least 99.07%, at least 99.12%, at least 99.18%, at least 99.23%, at least 99.29%, at least 99.34%, at least 99.39%, at least 99.45%, at least 99.50%, at least 99.55%, at least 99.61%, at least 99.66%, at least 99.71%, at least 99.77%, at least 99.82%, at least 99.88%, at least 99.93%, at least 99.98%, or 100% identical) to SEQ ID NO: 34, (f) a nucleic acid sequence that is at least 90.83% identical (e.g., at least 90.87%, at least 90.90%, at least 90.94%, at least 90.97%, at least 91.01%, at least 91.04%, at least 91.08%, at least 91.11%, at least 91.15%, at least 91.18%, at least 91.22%, at least 91.25%, at least 91.29%, at least 91.32%, at least 91.36%, at least 91.39%, at least 91.43%, at least 91.46%, at least 91.50%, at least 91.53%, at least 91.57%, at least 91.60%, at least 91.64%, at least 91.67%, at least 91.71%, at least 91.75%, at least 91.78%, at least 91.82%, at least 91.85%, at least 91.89%, at least 91.92%, at least 91.96%, at least 91.99%, at least 92.03%, at least 92.06%, at least 92.10%, at least 92.13%, at least 92.17%, at least 92.20%, at least 92.24%, at least 92.27%, at least 92.31%, at least 92.34%, at least 92.38%, at least 92.41%, at least 92.45%, at least 92.48%, at least 92.52%, at least 92.55%, at least 92.59%, at least 92.63%, at least 92.66%, at least 92.70%, at least 92.73%, at least 92.77%, at least 92.80%, at least 92.84%, at least 92.87%, at least 92.91%, at least 92.94%, at least 92.98%, at least 93.01%, at least 93.05%, at least 93.08%, at least 93.12%, at least 93.15%, at least 93.19%, at least 93.22%, at least 93.26%, at least 93.29%, at least 93.33%, at least 93.36%, at least 93.40%, at least 93.43%, at least 93.47%, at least 93.51%, at least 93.54%, at least 93.58%, at least 93.61%, at least 93.65%, at least 93.68%, at least 93.72%, at least 93.75%, at least 93.79%, at least 93.82%, at least 93.86%, at least 93.89%, at least 93.93%, at least 93.96%, at least 94.00%, at least 94.03%, at least 94.07%, at least 94.10%, at least 94.14%, at least 94.17%, at least 94.21%, at least 94.24%, at least 94.28%, at least 94.31%, at least 94.35%, at least 94.39%, at least 94.42%, at least 94.46%, at least 94.49%, at least 94.53%, at least 94.56%, at least 94.60%, at least 94.63%, at least 94.67%, at least 94.70%, at least 94.74%, at least 94.77%, at least 94.81%, at least 94.84%, at least 94.88%, at least 94.91%, at least 94.95%, at least 94.98%, at least 95.02%, at least 95.05%, at least 95.09%, at least 95.12%, at least 95.16%, at least 95.19%, at least 95.23%, at least 95.27%, at least 95.30%, at least 95.34%, at least 95.37%, at least 95.41%, at least 95.44%, at least 95.48%, at least 95.51%, at least 95.55%, at least 95.58%, at least 95.62%, at least 95.65%, at least 95.69%, at least 95.72%, at least 95.76%, at least 95.79%, at least 95.83%, at least 95.86%, at least 95.90%, at least 95.93%, at least 95.97%, at least 96.00%, at least 96.04%, at least 96.07%, at least 96.11%, at least 96.15%, at least 96.18%, at least 96.22%, at least 96.25%, at least 96.29%, at least 96.32%, at least 96.36%, at least 96.39%, at least 96.43%, at least 96.46%, at least 96.50%, at least 96.53%, at least 96.57%, at least 96.60%, at least 96.64%, at least 96.67%, at least 96.71%, at least 96.74%, at least 96.78%, at least 96.81%, at least 96.85%, at least 96.88%, at least 96.92%, at least 96.95%, at least 96.99%, at least 97.03%, at least 97.06%, at least 97.10%, at least 97.13%, at least 97.17%, at least 97.20%, at least 97.24%, at least 97.27%, at least 97.31%, at least 97.34%, at least 97.38%, at least 97.41%, at least 97.45%, at least 97.48%, at least 97.52%, at least 97.55%, at least 97.59%, at least 97.62%, at least 97.66%, at least 97.69%, at least 97.73%, at least 97.76%, at least 97.80%, at least 97.83%, at least 97.87%, at least 97.90%, at least 97.94%, at least 97.98%, at least 98.01%, at least 98.05%, at least 98.08%, at least 98.12%, at least 98.15%, at least 98.19%, at least 98.22%, at least 98.26%, at least 98.29%, at least 98.33%, at least 98.36%, at least 98.40%, at least 98.43%, at least 98.47%, at least 98.50%, at least 98.54%, at least 98.57%, at least 98.61%, at least 98.64%, at least 98.68%, at least 98.71%, at least 98.75%, at least 98.78%, at least 98.82%, at least 98.86%, at least 98.89%, at least 98.93%, at least 98.96%, at least 99.00%, at least 99.03%, at least 99.07%, at least 99.10%, at least 99.14%, at least 99.17%, at least 99.21%, at least 99.24%, at least 99.28%, at least 99.31%, at least 99.35%, at least 99.38%, at least 99.42%, at least 99.45%, at least 99.49%, at least 99.52%, at least 99.56%, at least 99.59%, at least 99.63%, at least 99.66%, at least 99.70%, at least 99.74%, at least 99.77%, at least 99.81%, at least 99.84%, at least 99.88%, at least 99.91%, at least 99.95%, at least 99.98%, or 100% identical) to SEQ ID NO: 35, and (g) a nucleic acid sequence that is at least 82.5% identical (e.g., at least 82.56%, at least 82.61%, at least 82.67%, at least 82.73%, at least 82.79%, at least 82.84%, at least 82.90%, at least 82.96%, at least 83.02%, at least 83.07%, at least 83.13%, at least 83.19%, at least 83.25%, at least 83.30%, at least 83.36%, at least 83.42%, at least 83.48%, at least 83.53%, at least 83.59%, at least 83.65%, at least 83.71%, at least 83.76%, at least 83.82%, at least 83.88%, at least 83.94%, at least 83.99%, at least 84.05%, at least 84.11%, at least 84.17%, at least 84.22%, at least 84.28%, at least 84.34%, at least 84.40%, at least 84.45%, at least 84.51%, at least 84.57%, at least 84.63%, at least 84.68%, at least 84.74%, at least 84.80%, at least 84.86%, at least 84.91%, at least 84.97%, at least 85.03%, at least 85.09%, at least 85.14%, at least 85.20%, at least 85.26%, at least 85.32%, at least 85.37%, at least 85.43%, at least 85.49%, at least 85.55%, at least 85.60%, at least 85.66%, at least 85.72%, at least 85.78%, at least 85.83%, at least 85.89%, at least 85.95%, at least 86.01%, at least 86.06%, at least 86.12%, at least 86.18%, at least 86.24%, at least 86.29%, at least 86.35%, at least 86.41%, at least 86.47%, at least 86.52%, at least 86.58%, at least 86.64%, at least 86.70%, at least 86.75%, at least 86.81%, at least 86.87%, at least 86.93%, at least 86.98%, at least 87.04%, at least 87.10%, at least 87.16%, at least 87.21%, at least 87.27%, at least 87.33%, at least 87.39%, at least 87.44%, at least 87.50%, at least 87.56%, at least 87.61%, at least 87.67%, at least 87.73%, at least 87.79%, at least 87.84%, at least 87.90%, at least 87.96%, at least 88.02%, at least 88.07%, at least 88.13%, at least 88.19%, at least 88.25%, at least 88.30%, at least 88.36%, at least 88.42%, at least 88.48%, at least 88.53%, at least 88.59%, at least 88.65%, at least 88.71%, at least 88.76%, at least 88.82%, at least 88.88%, at least 88.94%, at least 88.99%, at least 89.05%, at least 89.11%, at least 89.17%, at least 89.22%, at least 89.28%, at least 89.34%, at least 89.40%, at least 89.45%, at least 89.51%, at least 89.57%, at least 89.63%, at least 89.68%, at least 89.74%, at least 89.80%, at least 89.86%, at least 89.91%, at least 89.97%, at least 90.03%, at least 90.09%, at least 90.14%, at least 90.20%, at least 90.26%, at least 90.32%, at least 90.37%, at least 90.43%, at least 90.49%, at least 90.55%, at least 90.60%, at least 90.66%, at least 90.72%, at least 90.78%, at least 90.83%, at least 90.89%, at least 90.95%, at least 91.01%, at least 91.06%, at least 91.12%, at least 91.18%, at least 91.24%, at least 91.29%, at least 91.35%, at least 91.41%, at least 91.47%, at least 91.52%, at least 91.58%, at least 91.64%, at least 91.70%, at least 91.75%, at least 91.81%, at least 91.87%, at least 91.93%, at least 91.98%, at least 92.04%, at least 92.10%, at least 92.16%, at least 92.21%, at least 92.27%, at least 92.33%, at least 92.39%, at least 92.44%, at least 92.50%, at least 92.56%, at least 92.61%, at least 92.67%, at least 92.73%, at least 92.79%, at least 92.84%, at least 92.90%, at least 92.96%, at least 93.02%, at least 93.07%, at least 93.13%, at least 93.19%, at least 93.25%, at least 93.30%, at least 93.36%, at least 93.42%, at least 93.48%, at least 93.53%, at least 93.59%, at least 93.65%, at least 93.71%, at least 93.76%, at least 93.82%, at least 93.88%, at least 93.94%, at least 93.99%, at least 94.05%, at least 94.11%, at least 94.17%, at least 94.22%, at least 94.28%, at least 94.34%, at least 94.40%, at least 94.45%, at least 94.51%, at least 94.57%, at least 94.63%, at least 94.68%, at least 94.74%, at least 94.80%, at least 94.86%, at least 94.91%, at least 94.97%, at least 95.03%, at least 95.09%, at least 95.14%, at least 95.20%, at least 95.26%, at least 95.32%, at least 95.37%, at least 95.43%, at least 95.49%, at least 95.55%, at least 95.60%, at least 95.66%, at least 95.72%, at least 95.78%, at least 95.83%, at least 95.89%, at least 95.95%, at least 96.01%, at least 96.06%, at least 96.12%, at least 96.18%, at least 96.24%, at least 96.29%, at least 96.35%, at least 96.41%, at least 96.47%, at least 96.52%, at least 96.58%, at least 96.64%, at least 96.70%, at least 96.75%, at least 96.81%, at least 96.87%, at least 96.93%, at least 96.98%, at least 97.04%, at least 97.10%, at least 97.16%, at least 97.21%, at least 97.27%, at least 97.33%, at least 97.39%, at least 97.44%, at least 97.50%, at least 97.56%, at least 97.61%, at least 97.67%, at least 97.73%, at least 97.79%, at least 97.84%, at least 97.90%, at least 97.96%, at least 98.02%, at least 98.07%, at least 98.13%, at least 98.19%, at least 98.25%, at least 98.30%, at least 98.36%, at least 98.42%, at least 98.48%, at least 98.53%, at least 98.59%, at least 98.65%, at least 98.71%, at least 98.76%, at least 98.82%, at least 98.88%, at least 98.94%, at least 98.99%, at least 99.05%, at least 99.11%, at least 99.17%, at least 99.22%, at least 99.28%, at least 99.34%, at least 99.40%, at least 99.45%, at least 99.51%, at least 99.57%, at least 99.63%, at least 99.68%, at least 99.74%, at least 99.80%, at least 99.86%, at least 99.91%, at least 99.97%, or 100% identical) to SEQ ID NO: 36.

The adenovirus or adenoviral vector can comprise one, two, three, four, five, six or all seven of the aforementioned sequences alone or in any combination. In this respect, the adenovirus or adenoviral vector can comprise any combination of any two of the aforementioned sequences, any combination of any three of the aforementioned sequences, any combination of any four of the aforementioned sequences, any combination of any five of the aforementioned sequences, or all six of the aforementioned sequences. For example, the adenovirus or adenoviral vector can comprise a nucleic acid sequence that is at least 98.7% identical to SEQ ID NO: 30. The adenovirus or adenoviral vector can comprise a nucleic acid sequence that is at least 81.25% identical to SEQ ID NO: 34 and a nucleic acid sequence that is at least 82.50% identical to SEQ ID NO: 36. The adenovirus or adenoviral vector can comprise a nucleic acid sequence that is at least 99.4% identical to SEQ ID NO: 32, a nucleic acid sequence that is at least 99.1% identical to SEQ ID NO: 33, a nucleic acid sequence that is at least 81.25% identical to SEQ ID NO: 34, and a nucleic acid sequence that is at least 82.5% identical to SEQ ID NO: 36. The adenovirus or adenoviral vector can comprise (a) the nucleic acid sequence of SEQ ID NO: 30, (b) the nucleic acid sequence of SEQ ID NO: 33, (c) the nucleic acid sequence of SEQ ID NO: 34, (d) the nucleic acid sequence of SEQ ID NO: 35, or (e) the nucleic acid sequence of SEQ ID NO: 36. The adenovirus or adenoviral vector can comprise (a) a nucleic acid sequence that is at least 99.4% identical to SEQ ID NO: 32, (b) a nucleic acid sequence that is at least 99.1% identical to SEQ ID NO: 33, (c) a nucleic acid sequence that is at least 81.25% identical to SEQ ID NO: 34, (d) a nucleic acid sequence that is at least 90.83% identical to SEQ ID NO: 35, and (e) a nucleic acid sequence that is at least 82.5% identical to SEQ ID NO: 36. The adenovirus or adenoviral vector can comprise (a) the nucleic acid sequence SEQ ID NO: 31, (b) the nucleic acid sequence of SEQ ID NO: 33, (c) the nucleic acid sequence of SEQ ID NO: 34, (d) the nucleic acid sequence of SEQ ID NO: 35, and (e) the nucleic acid sequence of SEQ ID NO: 36.

In another embodiment, the adenovirus or adenoviral vector comprises one or more of the following nucleic acid sequences: (a) a nucleic acid sequence comprising at least 162 contiguous nucleotides of SEQ ID NO: 30, (b) a nucleic acid sequence comprising at least 162 contiguous nucleotides of SEQ ID NO: 31, (c) a nucleic acid sequence comprising at least 121 contiguous nucleotides of SEQ ID NO: 32, (d) a nucleic acid sequence comprising at least 531 contiguous nucleotides of SEQ ID NO: 33, (e) a nucleic acid sequence comprising at least 156 contiguous nucleotides of SEQ ID NO: 34, (f) a nucleic acid sequence comprising at least 192 contiguous nucleotides of SEQ ID NO: 35, or (g) a nucleic acid sequence comprising at least 84 contiguous nucleotides of SEQ ID NO: 36.

The adenovirus or adenoviral vector can comprise a nucleic acid sequence comprising at least 162 (e.g., 165 or more, 170 or more, 190 or more, 200 or more, 250 or more, or 300 or more) contiguous nucleotides of SEQ ID NO: 30, but no more than 399 (e.g., 398 or less, 350 or less, or 275 or less) contiguous nucleotides of SEQ ID NO: 30. Preferably, the adenovirus or adenoviral vector comprises a nucleic acid sequence comprising 162 to 300 contiguous nucleotides (e.g., 163, 175, 200, 250, or 275 contiguous nucleotides), or 162 to 200 contiguous nucleotides (e.g., 164, 166, 167, 168, 169, 171, 172, 173, 174, 176, 177, 178, 179, 180, 181, 182, 183, 184, 185, 186, 187, 188, 189, 191, 192, 193, 194, 195, 196, 197, 198, or 199 contiguous nucleotides) of SEQ ID NO: 30, or a range defined by any two of the foregoing values.

The adenovirus or adenoviral vector can comprise a nucleic acid sequence comprising at least 162 (e.g., 165 or more, 170 or more, 190 or more, 200 or more, 250 or more, or 300 or more) contiguous nucleotides of SEQ ID NO: 31, but no more than 399 (e.g., 398 or less, 350 or less, or 275 or less) contiguous nucleotides of SEQ ID NO: 31. Preferably, the adenovirus or adenoviral vector comprises a nucleic acid sequence comprising 162 to 300 contiguous nucleotides (e.g., 163, 175, 200, 250, or 275 contiguous nucleotides), or 162 to 200 contiguous nucleotides (e.g., 164, 166, 167, 168, 169, 171, 172, 173, 174, 176, 177, 178, 179, 180, 181, 182, 183, 184, 185, 186, 187, 188, 189, 191, 192, 193, 194, 195, 196, 197, 198, or 199 contiguous nucleotides) of SEQ ID NO: 31, or a range defined by any two of the foregoing values.

The adenovirus or adenoviral vector can comprise a nucleic acid sequence comprising at least 121 (e.g., 125 or more, 130 or more, 150 or more, 200 or more, 250 or more, or 300 or more) contiguous nucleotides of SEQ ID NO: 32, but no more than 399 (e.g., 398 or less, 350 or less, or 275 or less) contiguous nucleotides of SEQ ID NO: 32. Preferably, the adenovirus or adenoviral vector comprises a nucleic acid sequence comprising 121 to 300 contiguous nucleotides (e.g., 125, 150, 175, 200, 250, or 275 contiguous nucleotides), 121 to 200 contiguous nucleotides (e.g., 130, 140, 145, 160, 165, 170, 180, 185, 190, 195, or 199 contiguous nucleotides), or 121 to 150 contiguous nucleotides (e.g., 122, 123, 124, 125, 126, 127, 128, 129, 130, 131, 132, 133, 134, 135, 136, 137, 138, 139, 140, 141, 142, 143, 144, 145, 146, 147, 148, or 149 contiguous nucleotides) of SEQ ID NO: 32, or a range defined by any two of the foregoing values.

The adenovirus or adenoviral vector can comprise a nucleic acid sequence comprising at least 531 (e.g., 540 or more, 600 or more, 700 or more, 800 or more, 900 or more, or 1,000 or more) contiguous nucleotides of SEQ ID NO: 33, but no more than 3168 (e.g., 3,100 or less, 3,000 or less, 2,500 or less, 2,000 or less, or 1,500 or less) contiguous nucleotides of SEQ ID NO: 33. Preferably, the adenovirus or adenoviral vector comprises a nucleic acid sequence comprising 531 to 2,000 contiguous nucleotides (e.g., 550, 600, 700, 1,000, 1,200, 1,500, or 1,700 contiguous nucleotides), 531 to 1,000 contiguous nucleotides (e.g., 535, 575, 600, 650, 675, 725, 750, 800, 850, 900, or 950 contiguous nucleotides), or 531 to 800 contiguous nucleotides (e.g., 540, 545, 550, 560, 565, 570, 580, 585, 590, 595, 615, 625, 630, 640, 660, 665, 670, 680, 685, 690, 695, 705, 715, 730, 740, 755, 760, 765, 770, 775, 780, 785, 790, 795, or 799 contiguous nucleotides) of SEQ ID NO: 33, or a range defined by any two of the foregoing values.

The adenovirus or adenoviral vector can comprise a nucleic acid sequence comprising at least 156 (e.g., 160 or more, 200 or more, 225 or more, 235 or more, 250 or more, 300 or more, 350 or more, 400 or more, 450 or more, or 500 or more) contiguous nucleotides of SEQ ID NO: 34, but no more than 1,863 (e.g., 1,800 or less, 1,500 or less, 1,200 or less, 1,000 or less, 850 or less, 800 or less, 750 or less, or 700 or less) contiguous nucleotides of SEQ ID NO: 34. Preferably, the adenovirus or adenoviral vector comprises a nucleic acid sequence comprising 156 to 1,500 contiguous nucleotides (e.g., 175, 210, 225, 245, 255, 265, 275, 290, 300, 400, 500, 600, 700, 800, 900, 1,000, or 1,200 contiguous nucleotides), 156 to 1,000 contiguous nucleotides (e.g., 165, 180, 185, 195, 205, 230, 240, 260, 270, 295, 350, 450, 550, 650, 750, 850, or 950 contiguous nucleotides), or 156 to 500 contiguous nucleotides (e.g., 199, 230, 235, 290, 305, 310, 315, 325, 340, 345, 360, 365, 370, 375, 380, 385, 390, 395, 405, 425, 430, 440, 455, 460, 465, 470, 475, 480, 485, 490, 495, or 499 contiguous nucleotides) of SEQ ID NO: 34, or a range defined by any two of the foregoing values.

The adenovirus or adenoviral vector can comprise a nucleic acid sequence comprising at least 192 (e.g., 200 or more, 300 or more, 400 or more, 500 or more, or 600 or more) contiguous nucleotides of SEQ ID NO: 35, but no more than 2841 (2,800 or less, 2,500 or less, 2,000 or less, 1,800 or less, or 1,500 or less) contiguous nucleotides of SEQ ID NO: 35. Preferably, the adenovirus or adenoviral vector comprises a nucleic acid sequence comprising 192 to 2,000 contiguous nucleotides (e.g., 200, 250, 300, 350, 400, 450, 500, 550, 600, 650, 700, 800, 900, 1,000, 1,200, 1,500, 1,700, or 1,900 contiguous nucleotides), 192 to 1,000 contiguous nucleotides (e.g., 275, 375, 475, 575, 675, 775, 875, or 975 contiguous nucleotides), or 192 to 500 contiguous nucleotides (e.g., 220, 235, 240, 255, 260, 270, 280, 285, 290, 295, 299, 305, 315, 330, 335, 340, 345, 355, 360, 365, 370, 385, 390, 395, 399, 405, 415, 430, 435, 440, 445, 455, 460, 465, 470, 485, 490, 495, 499 contiguous nucleotides) of SEQ ID NO: 35, or a range defined by any two of the foregoing values.

The adenovirus or adenoviral vector can comprise a nucleic acid sequence comprising at least 84 (e.g., 90 or more, 100 or more, 200 or more, 300 or more, 500 or more, 700 or more, or 900 or more) contiguous nucleotides of SEQ ID NO: 36, but no more than 1,740 (1,700 or less, 1,500 or less, 1,200 or less, or 1,000 or less) contiguous nucleotides of SEQ ID NO: 36. Preferably, the adenovirus or adenoviral vector comprises a nucleic acid sequence comprising 84 to 1,200 contiguous nucleotides (e.g., 95, 100, 200, 400, 600, 800, 1,000, or 1,200 contiguous nucleotides), 84 to 1,000 contiguous nucleotides (e.g., 95, 150, 195, 250, 350, 450, 550, 650, 750, 850, or 950 contiguous nucleotides), or 84 to 500 contiguous nucleotides (e.g., 90, 99, 115, 125, 130, 145, 155, 165, 175, 190, 225, 230, 240, 255, 260, 265, 270, 275, 315, 325, 330, 340, 355, 360, 365, 370, 375, 380, 385, 390, 395, 415, 425, 430, 440, 455, 460, 465, 470, 475, 480, 485, 490, 495, or 499 contiguous nucleotides) of SEQ ID NO: 36, or a range defined by any two of the foregoing values.

The adenovirus or adenoviral vector can comprise one, two, three, four, five, six, or all seven of the aforementioned sequences alone, or in any combination. In this respect, the adenovirus or adenoviral vector can comprise any combination of any two of the aforementioned sequences, any combination of any three of the aforementioned sequences, any combination of any four of the aforementioned sequences, any combination of any five of the aforementioned sequences, any combination of any six of the aforementioned sequences, or all seven of the aforementioned sequences. For example, the adenovirus or adenoviral vector can comprise a nucleic acid sequence comprising at least 162 contiguous nucleotides of SEQ ID NO: 30. The adenovirus or adenoviral vector can comprise a nucleic acid sequence comprising at least 121 contiguous nucleotides of SEQ ID NO: 32, a nucleic acid sequence comprising at least 156 contiguous nucleotides of SEQ ID NO: 34, and a nucleic acid sequence comprising at least 84 contiguous nucleotides of SEQ ID NO: 36. The adenovirus or adenoviral vector can comprise a nucleic acid sequence comprising at least 162 contiguous nucleotides of SEQ ID NO: 31, a nucleic acid sequence comprising at least 531 contiguous nucleotides of SEQ ID NO: 33, and a nucleic acid sequence comprising at least 192 contiguous nucleotides of SEQ ID NO: 35. The adenovirus or adenoviral vector can comprise a nucleic acid sequence comprising at least 121 contiguous nucleotides of SEQ ID NO: 32, and a nucleic acid sequence comprising at least 531 contiguous nucleotides of SEQ ID NO: 33. The adenovirus or adenoviral vector can comprise (a) a nucleic acid sequence comprising at least 162 contiguous nucleotides of SEQ ID NO: 30 or SEQ ID NO: 31, (b) a nucleic acid sequence comprising at least 531 contiguous nucleotides of SEQ ID NO: 33, (c) a nucleic acid sequence comprising at least 156 contiguous nucleotides of SEQ ID NO: 34, (d) a nucleic acid sequence comprising at least 192 contiguous nucleotides of SEQ ID NO: 35, and (e) a nucleic acid sequence comprising at least 84 contiguous nucleotides of SEQ ID NO: 36.

In another embodiment, the adenovirus or adenoviral vector comprises one or more of the following amino acid sequences: (a) an amino acid sequence that is at least 85% identical (e.g., at least 91.67%, at least 98.33%, or 100% identical) to SEQ ID NO: 38, (b) an amino acid sequence that is at least 80% identical (e.g., at least 86.25%, at least 92.50%, at least 98.75%, or 100% identical) to SEQ ID NO: 39, and (c) an amino acid sequence that is at least 80% identical (e.g., at least 80.25%, at least 86.50%, at least 92.75%, at least 99.00%, or 100% identical) to SEQ ID NO: 40.

The adenovirus or adenoviral vector can comprise one, two, or all three of the aforementioned amino acid sequences alone or in any combination. In this respect, the adenovirus or adenoviral vector can comprise one of the aforementioned sequences, any combination of any two of the aforementioned sequences, or all three of the aforementioned sequences. For example, the adenovirus or adenoviral vector can comprise an amino acid sequence that is at least 85% identical to SEQ ID NO: 38. The adenovirus or adenoviral vector can comprise an amino acid sequence that is at least 85% identical to SEQ ID NO: 38, and an amino acid sequence that is at least 80% identical to SEQ ID NO: 39. The adenovirus or adenoviral vector can comprise (a) an amino acid sequence that is at least 85% identical to SEQ ID NO: 38, (b) an amino acid sequence that is at least 80% identical to SEQ ID NO: 39, and (c) an amino acid sequence that is at least 80% identical to SEQ ID NO: 40. The adenovirus or adenoviral vector can comprise (a) the amino acid sequence of SEQ ID NO: 38, (b) the amino acid sequence of SEQ ID NO: 39, or (c) the amino acid sequence of SEQ ID NO: 40. The adenovirus or adenoviral vector can comprise (a) the amino acid sequence of SEQ ID NO: 38, (b) the amino acid sequence of SEQ ID NO: 39, and (c) the amino acid sequence of SEQ ID NO: 40.

In another embodiment, the adenovirus or adenoviral vector comprises one or more of the following amino acid sequences: (a) an amino acid sequence that is at least 99% identical (e.g., at least 99% or 100% identical) to SEQ ID NO: 42, (b) an amino acid sequence that is at least 81.4% identical (e.g., at least 81.56%, at least 81.72%, at least 81.88%, at least 82.04%, at least 82.21%, at least 82.37%, at least 82.53%, at least 82.69%, at least 82.85%, at least 83.01%, at least 83.17%, at least 83.33%, at least 83.49%, at least 83.65%, at least 83.82%, at least 83.98%, at least 84.14%, at least 84.30%, at least 84.46%, at least 84.62%, at least 84.78%, at least 84.94%, at least 85.10%, at least 85.26%, at least 85.43%, at least 85.59%, at least 85.75%, at least 85.91%, at least 86.07%, at least 86.23%, at least 86.39%, at least 86.55%, at least 86.71%, at least 86.88%, at least 87.04%, at least 87.20%, at least 87.36%, at least 87.52%, at least 87.68%, at least 87.84%, at least 88.00%, at least 88.16%, at least 88.32%, at least 88.49%, at least 88.65%, at least 88.81%, at least 88.97%, at least 89.13%, at least 89.29%, at least 89.45%, at least 89.61%, at least 89.77%, at least 89.93%, at least 90.10%, at least 90.26%, at least 90.42%, at least 90.58%, at least 90.74%, at least 90.90%, at least 91.06%, at least 91.22%, at least 91.38%, at least 91.54%, at least 91.71%, at least 91.87%, at least 92.03%, at least 92.19%, at least 92.35%, at least 92.51%, at least 92.67%, at least 92.83%, at least 92.99%, at least 93.16%, at least 93.32%, at least 93.48%, at least 93.64%, at least 93.80%, at least 93.96%, at least 94.12%, at least 94.28%, at least 94.44%, at least 94.60%, at least 94.77%, at least 94.93%, at least 95.09%, at least 95.25%, at least 95.41%, at least 95.57%, at least 95.73%, at least 95.89%, at least 96.05%, at least 96.21%, at least 96.38%, at least 96.54%, at least 96.70%, at least 96.86%, at least 97.02%, at least 97.18%, at least 97.34%, at least 97.50%, at least 97.66%, at least 97.83%, at least 97.99%, at least 98.15%, at least 98.31%, at least 98.47%, at least 98.63%, at least 98.79%, at least 98.95%, at least 99.11%, at least 99.27%, at least 99.44%, at least 99.60%, at least 99.76%, at least 99.92%, or 100% identical to) to SEQ ID NO: 44, (c) an amino acid sequence that is at least 91.3% identical (e.g., at least 91.41%, at least 91.51%, at least 91.62%, at least 91.72%, at least 91.83%, at least 91.93%, at least 92.04%, at least 92.14%, at least 92.25%, at least 92.36%, at least 92.46%, at least 92.57%, at least 92.67%, at least 92.78%, at least 92.88%, at least 92.99%, at least 93.10%, at least 93.20%, at least 93.31%, at least 93.41%, at least 93.52%, at least 93.62%, at least 93.73%, at least 93.83%, at least 93.94%, at least 94.05%, at least 94.15%, at least 94.26%, at least 94.36%, at least 94.47%, at least 94.57%, at least 94.68%, at least 94.78%, at least 94.89%, at least 95.00%, at least 95.10%, at least 95.21%, at least 95.31%, at least 95.42%, at least 95.52%, at least 95.63%, at least 95.74%, at least 95.84%, at least 95.95%, at least 96.05%, at least 96.16%, at least 96.26%, at least 96.37%, at least 96.47%, at least 96.58%, at least 96.69%, at least 96.79%, at least 96.90%, at least 97.00%, at least 97.11%, at least 97.21%, at least 97.32%, at least 97.42%, at least 97.53%, at least 97.64%, at least 97.74%, at least 97.85%, at least 97.95%, at least 98.06%, at least 98.16%, at least 98.27%, at least 98.37%, at least 98.48%, at least 98.59%, at least 98.69%, at least 98.80%, at least 98.90%, at least 99.01%, at least 99.11%, at least 99.22%, at least 99.33%, at least 99.43%, at least 99.54%, at least 99.64%, at least 99.75%, at least 99.85%, at least 99.96%, or 100% identical) to SEQ ID NO: 45, and (d) an amino acid sequence that is at least 83.4% identical (e.g., at least 83.57%, at least 83.74%, at least 83.92%, at least 84.09%, at least 84.26%, at least 84.43%, at least 84.61%, at least 84.78%, at least 84.95%, at least 85.12%, at least 85.30%, at least 85.47%, at least 85.64%, at least 85.81%, at least 85.99%, at least 86.16%, at least 86.33%, at least 86.50%, at least 86.68%, at least 86.85%, at least 87.02%, at least 87.19%, at least 87.37%, at least 87.54%, at least 87.71%, at least 87.88%, at least 88.06%, at least 88.23%, at least 88.40%, at least 88.57%, at least 88.74%, at least 88.92%, at least 89.09%, at least 89.26%, at least 89.43%, at least 89.61%, at least 89.78%, at least 89.95%, at least 90.12%, at least 90.30%, at least 90.47%, at least 90.64%, at least 90.81%, at least 90.99%, at least 91.16%, at least 91.33%, at least 91.50%, at least 91.68%, at least 91.85%, at least 92.02%, at least 92.19%, at least 92.37%, at least 92.54%, at least 92.71%, at least 92.88%, at least 93.06%, at least 93.23%, at least 93.40%, at least 93.57%, at least 93.74%, at least 93.92%, at least 94.09%, at least 94.26%, at least 94.43%, at least 94.61%, at least 94.78%, at least 94.95%, at least 95.12%, at least 95.30%, at least 95.47%, at least 95.64%, at least 95.81%, at least 95.99%, at least 96.16%, 96.33%, at least 96.50%, at least 96.68%, at least 96.85%, at least 97.02%, at least 97.19%, at least 97.37%, at least 97.54%, at least 97.71%, at least 97.88%, at least 98.06%, at least 98.23%, at least 98.40%, at least 98.57%, at least 98.74%, at least 98.92%, at least 99.09%, at least 99.26%, at least 99.43%, at least 99.61%, at least 99.78%, at least 99.95%, or 100% identical) to SEQ ID NO: 46.

The adenovirus or adenoviral vector can comprise one, two, three, or all four of the aforementioned amino acid sequences alone or in any combination. In this respect, the adenovirus or adenoviral vector can comprise any combination of any two of the aforementioned sequences, any combination of any three of the aforementioned sequences, or all four of the aforementioned sequences. For example, the adenovirus or adenoviral vector can comprise an amino acid sequence that is at least 99% identical to SEQ ID NO: 42. The adenovirus or adenoviral vector can comprise an amino acid sequence that is at least 99% identical to SEQ ID NO: 42, and an amino acid sequence that is at least 91.3% identical to SEQ ID NO: 45. The adenovirus or adenoviral vector can comprise an amino acid sequence that is at least 99% identical to SEQ ID NO: 42, an amino acid sequence that is at least 81.4% identical to SEQ ID NO: 44, and an amino acid sequence that is at least 91.3% identical to SEQ ID NO: 45. The adenovirus or adenoviral vector can comprise (a) the amino acid sequence of SEQ ID NO: 42, (b) the amino acid sequence of SEQ ID NO: 44, (c) the amino acid sequence of SEQ ID NO: 45, or (d) the amino acid sequence of SEQ ID NO: 46. The adenovirus or adenoviral vector can comprise (a) an amino acid sequence that is at least 99% identical to SEQ ID NO: 42, (b) an amino acid sequence that is at least 81.4% identical to SEQ ID NO: 44, (c) an amino acid sequence that is at least 91.3% identical to SEQ ID NO: 45, and (d) an amino acid sequence that is at least 83.4% identical to SEQ ID NO: 46. The adenovirus or adenoviral vector can comprise (a) the amino acid sequence of SEQ ID NO: 42, (b) the amino acid sequence of SEQ ID NO: 44, (c) the amino acid sequence of SEQ ID NO: 45, and (d) the amino acid sequence of SEQ ID NO: 46.

In another embodiment, the adenovirus or adenoviral vector comprises one or more of the following amino acid sequences: (a) an amino acid sequence comprising at least 89 contiguous amino acid residues of SEQ ID NO: 42, (b) an amino acid sequence comprising at least 114 contiguous amino acid residues of SEQ ID NO: 44, (c) an amino acid sequence comprising at least 231 contiguous amino acid residues of SEQ ID NO: 45, and (d) an amino acid sequence comprising at least 30 contiguous amino acid residues of SEQ ID NO: 46.

The adenovirus or adenoviral vector can comprise an amino acid sequence comprising at least 89 (e.g., 90 or more, 100 or more, or 110 or more) contiguous amino acid residues of SEQ ID NO: 42, but no more than 133 (e.g., 130 or less, 125 or less, 120 or less, or 115 or less) contiguous amino acid residues of SEQ ID NO: 42. Preferably, the adenovirus or adenoviral vector comprises an amino acid sequence comprising 89 to 130 contiguous amino acid residues (e.g., 90, 100, 110, 115, 120, or 125 contiguous amino acid residues) of SEQ ID NO: 42, 89 to 115 contiguous amino acid residues of SEQ ID NO: 42 (e.g., 95, 110, or 112 contiguous amino acid residues), or 89 to 100 contiguous amino acid residues (e.g., 89, 90, 91, 92, 93, 94, 95, 96, 97, 98, or 99 contiguous amino acid residues) of SEQ ID NO: 42, or a range defined by any two of the foregoing values.

The adenovirus or adenoviral vector can comprise an amino acid sequence comprising at least 114 (e.g., 125 or more, 150 or more, 175 or more, 200 or more, 250 or more 250 or more, 275 or more, 300 or more, or 400 or more) contiguous amino acid residues of SEQ ID NO: 44, but no more than 621 (e.g., 620 or less, 600 or less, 550 or less, or 450 or less) contiguous amino acid residues of SEQ ID NO: 44. Preferably, the adenovirus or adenoviral vector comprises an acid sequence comprising 114 to 600 contiguous amino acid residues (e.g., 155, 180, 200, 250, 275, 300, 400, or 500 contiguous amino acid residues) of SEQ ID NO: 44, 114 to 500 contiguous amino acid residues of SEQ ID NO: 44 (e.g., 130, 160, 190, 220, 250, 280, 310, 340, 370, 375, 400, 425, 450, or 475 contiguous amino acid residues), or 114 to 300 contiguous amino acid residues (e.g., 165, 175, 185, 195, 199, 15, 225, 235, 245, 255, 265, 285, 295, 299, 315, 325, 335, 345, 355, 360, 365, 380, 385, 390, 395, or 399 contiguous amino acid residues) of SEQ ID NO: 44, or a range defined by any two of the foregoing values.

The adenovirus or adenoviral vector can comprise an amino acid sequence comprising at least 231 (e.g., 250 or more, 300 or more, 400 or more, or 500 or more) contiguous amino acid residues of SEQ ID NO: 45, but no more than 947 (e.g., 940 or less, 900 or less, 800 or less, 700 or less, or 600 or less) contiguous amino acid residues of SEQ ID NO: 45. Preferably, the adenovirus or adenoviral vector comprises an acid sequence comprising 231 to 800 contiguous amino acid residues (e.g., 250, 275, 290, 325, 350, 375, 390, 400, 500, 600, or 700 contiguous amino acid residues) of SEQ ID NO: 45, 231 to 600 contiguous amino acid residues (e.g., 235, 260, 285, 300, 335, 360, 375, 385, 395, 425, 445, 450, 465, 475, 525, 545, 550, 565 or 575 contiguous amino acid residues) of SEQ ID NO: 45, or 231 to 400 contiguous amino acid residues (e.g., 245, 255, 265, 285, 295, 299, 315, 345, 355, 365, 385, 389, 395, or 399 contiguous amino acid residues) of SEQ ID NO: 45, or a range defined by any two of the foregoing values.

The adenovirus or adenoviral vector can comprise an amino acid sequence comprising at least 30 (e.g., 50 or more, 75 or more, 100 or more, 200 or more, or 300 or more) contiguous amino acid residues of SEQ ID NO: 46, but no more than 580 (e.g., 575 or less, 550 or less, 500 or less, 450 or less, or 400 or less) contiguous amino acid residues of SEQ ID NO: 46. Preferably, the adenovirus or adenoviral vector comprises an acid sequence comprising 30 to 500 contiguous amino acid residues (e.g., 35, 55, 85, 105, 135, 155, 175, 195, 200, 205, 235, 250, 275, 295, 300, 305, 335, 350, 375, 395, 400, 405, 435, 450, 475, 495, or 499 contiguous amino acid residues) of SEQ ID NO: 46, 30 to 300 contiguous amino acid residues (e.g., 40, 60, 70, 90, 125, 140, 160, 170, 180, 190, 200, 210, 220, 230, 240, 250, 260, 270, 280, or 290 contiguous amino acid residues) of SEQ ID NO: 46, or 30 to 100 contiguous amino acid residues (e.g., 33, 34, 39, 42, 43, 44, 49, 52, 58, 59, 62, 68, 69, 72, 78, 79, 81, 84, 87, 88, 91, 92, 93, 94, 97, 98, or 99 contiguous amino acid residues) of SEQ ID NO: 46, or a range defined by any two of the foregoing values.

The adenovirus or adenoviral vector can comprise one, two, three, or all four of the aforementioned amino acid sequences alone or in any combination. In this respect, the adenovirus or adenoviral vector can comprise any combination of any two of the aforementioned sequences, any combination of any three of the aforementioned sequences, or all four of the aforementioned sequences. For example, the adenovirus or adenoviral vector can comprise an amino acid sequence comprising at least 89 contiguous amino acid residues of SEQ ID NO: 42. The adenovirus or adenoviral vector can comprise an amino acid sequence comprising at least 89 contiguous amino acid residues of SEQ ID NO: 42, and an amino acid sequence comprising at least 114 contiguous amino acid residues of SEQ ID NO: 44. The adenovirus or adenoviral vector can comprise (a) an amino acid sequence comprising at least 89 contiguous amino acid residues of SEQ ID NO: 42, (b) an amino acid sequence comprising at least 231 contiguous amino acid residues of SEQ ID NO: 45, and (c) an amino acid sequence comprising at least 30 contiguous amino acid residues of SEQ ID NO: 46. The adenovirus or adenoviral vector can comprise (a) an amino acid sequence comprising at least 114 contiguous amino acid residues of SEQ ID NO: 44, (b) an amino acid sequence comprising at least 231 contiguous amino acid residues of SEQ ID NO: 45, and (c) an amino acid sequence comprising at least 30 contiguous amino acid residues of SEQ ID NO: 46. The adenovirus or adenoviral vector can comprise (a) an amino acid sequence comprising at least 89 contiguous amino acid residues of SEQ ID NO: 42, (b) an amino acid sequence comprising at least 114 contiguous amino acid residues of SEQ ID NO: 44, (c) an amino acid sequence comprising at least 231 contiguous amino acid residues of SEQ ID NO: 45, and (d) an amino acid sequence comprising at least 30 contiguous amino acid residues of SEQ ID NO: 46.

In other embodiments, the adenovirus or adenoviral vector comprises one or more nucleic acid sequences that encode one or more of any of the aforementioned amino acid sequences, e.g., the amino acid sequences of any of SEQ ID NOs: 37-46 or any of the variants and/or portions thereof as described herein. For example, the adenovirus or adenoviral vector can comprise a nucleic acid sequence encoding an amino acid sequence that is at least 99.75% identical (e.g., at least 99.84%, at least 99.94%, or 100% identical) to SEQ ID NO: 43, or a nucleic acid sequence encoding an amino acid sequence that is at least 99.7% identical (e.g., at least 99.93% or 100% identical) to SEQ ID NO: 37.

In one embodiment, the adenovirus or adenoviral vector comprises one or more of the following nucleic acid sequences: (a) a nucleic acid sequence that is at least 97% identical (e.g., at least 98.20%, at least 99.41%, or 100% identical) to SEQ ID NO: 59, (b) a nucleic acid sequence that is at least 97.5% identical (e.g., at least 98.5%, at least 99.5%, or 100% identical) to SEQ ID NO: 60, (c) a nucleic acid sequence that is at least 80% identical (e.g., at least 82.22%, at least 84.44%, at least 86.67%, at least 88.89%, at least 91.11%, at least 93.33%, at least 95.56%, at least 97.78%, or 100% identical) to SEQ ID NO: 61, (d) a nucleic acid sequence that is at least 96% identical (e.g., at least 96.9%, at least 97.8%, at least 98.7%, at least 99.6%, or 100% identical) to SEQ ID NO: 62, and (e) a nucleic acid sequence that is at least 96% identical (e.g., at least 96.83% or 100% identical) to SEQ ID NO: 63.

The adenovirus or adenoviral vector can comprise one, two, three, four, or all five of the aforementioned sequences alone or in any combination. In this respect, the adenovirus or adenoviral vector can comprise any combination of any two of the aforementioned sequences, any combination of any three of the aforementioned sequences, any combination of any four of the aforementioned sequences, or all five of the aforementioned sequences. For example, the adenovirus or adenoviral vector can comprise a nucleic acid sequence that is at least 97% identical to SEQ ID NO: 59. The adenovirus or adenoviral vector can comprise a nucleic acid sequence that is at least 97.5% identical to SEQ ID NO: 60 and a nucleic acid sequence that is at least 80% identical to SEQ ID NO: 61. The adenovirus or adenoviral vector can comprise a nucleic acid sequence that is at least 97% identical to SEQ ID NO: 59, a nucleic acid sequence that is at least 80% identical to SEQ ID NO: 61, and a nucleic acid sequence that is at least 96% identical to SEQ ID NO: 63. The adenovirus or adenoviral vector can comprise (a) the nucleic acid sequence of SEQ ID NO: 59, (b) the nucleic acid sequence SEQ ID NO: 60, (c) the nucleic acid sequence of SEQ ID NO: 61, (d) the nucleic acid sequence of SEQ ID NO: 62, or (e) the nucleic acid sequence of SEQ ID NO: 63. The adenovirus or adenoviral vector can comprise (a) a nucleic acid sequence that is at least 97% identical to SEQ ID NO: 59, (b) a nucleic acid sequence that is at least 97.5% identical to SEQ ID NO: 60, (c) a nucleic acid sequence that is at least 80% identical to SEQ ID NO: 61, (d) a nucleic acid sequence that is at least 96% identical to SEQ ID NO: 62, and (e) a nucleic acid sequence that is at least 96% identical to SEQ ID NO: 63. The adenovirus or adenoviral vector can comprise (a) the nucleic acid sequence of SEQ ID NO: 59, (b) the nucleic acid sequence SEQ ID NO: 60, (c) the nucleic acid sequence of SEQ ID NO: 61, (d) the nucleic acid sequence of SEQ ID NO: 62, and (e) the nucleic acid sequence of SEQ ID NO: 63.

In another embodiment, the adenovirus or adenoviral vector comprises one or more of the following nucleic acid sequences: (a) a nucleic acid sequence that is at least 98.4% identical (e.g., at least 98.65%, at least 98.9%, at least 99.15%, at least 99.4%, at least 99.65%, at least 99.9%, or 100% identical) to SEQ ID NO: 64, (b) a nucleic acid sequence that is at least 99.01% identical (e.g., at least 99.04%, at least 99.07%, at least 99.10%, at least 99.14%, at least 99.17%, at least 99.20%, at least 99.23%, at least 99.26%, at least 99.29%, at least 99.33%, at least 99.36%, at least 99.39%, at least 99.42%, at least 99.45%, at least 99.48%, at least 99.52%, at least 99.55%, at least 99.58%, at least 99.61%, at least 99.64%, at least 99.67%, at least 99.70%, at least 99.74%, at least 99.77%, at least 99.80%, at least 99.83%, at least 99.86%, at least 99.89%, at least 99.93%, at least 99.96%, at least 99.99%, or 100% identical) to SEQ ID NO: 65, (c) a nucleic acid sequence that is at least 97.08% identical (e.g., at least 97.13%, at least 97.18%, at least 97.23%, at least 97.28%, at least 97.33%, at least 97.38%, at least 97.43%, at least 97.49%, at least 97.54%, at least 97.59%, at least 97.64%, at least 97.69%, at least 97.74%, at least 97.79%, at least 97.84%, at least 97.89%, at least 97.94%, at least 97.99%, at least 98.04%, at least 98.09%, at least 98.14%, at least 98.19%, at least 98.25%, at least 98.30%, at least 98.35%, at least 98.40%, at least 98.45%, at least 98.50%, at least 98.55%, at least 98.60%, at least 98.65%, at least 98.70%, at least 98.75%, at least 98.80%, at least 98.85%, at least 98.90%, at least 98.95%, at least 99.01%, at least 99.06%, at least 99.11%, at least 99.16%, at least 99.21%, at least 99.26%, at least 99.31%, at least 99.36%, at least 99.41%, at least 99.46%, at least 99.5%1, at least 99.56%, at least 99.61%, at least 99.66%, at least 99.71%, at least 99.76%, at least 99.82%, at least 99.87%, at least 99.92%, at least 99.97%, or 100% identical) to SEQ ID NO: 66, (d) a nucleic acid sequence that is at least 96.52% identical (e.g., at least 96.55%, at least 96.59%, at least 96.62%, at least 96.66%, at least 96.69%, at least 96.73%, at least 96.76%, at least 96.80%, at least 96.83%, at least 96.87%, at least 96.90%, at least 96.94%, at least 96.97%, at least 97.01%, at least 97.04%, at least 97.08%, at least 97.11%, at least 97.15%, at least 97.18%, at least 97.22%, at least 97.25%, at least 97.29%, at least 97.32%, at least 97.36%, at least 97.39%, at least 97.43%, at least 97.46%, at least 97.50%, at least 97.53%, at least 97.57%, at least 97.60%, at least 97.64%, at least 97.67%, at least 97.71%, at least 97.74%, at least 97.78%, at least 97.81%, at least 97.85%, at least 97.88%, at least 97.92%, at least 97.95%, at least 97.99%, at least 98.02%, at least 98.06%, at least 98.09%, at least 98.13%, at least 98.16%, at least at least 98.20%, at least 98.23%, at least 98.27%, at least 98.30%, at least 98.34%, at least 98.37%, at least at least 98.40%, at least 98.44%, at least 98.47%, at least 98.51%, at least 98.54%, at least 98.58%, at least 98.61%, at least 98.65%, at least 98.68%, at least 98.72%, at least 98.75%, at least 98.79%, at least 98.82%, at least 98.86%, at least 98.89%, at least 98.93%, at least 98.96%, at least 99.00%, at least 99.03%, at least 99.07%, at least 99.10%, at least 99.14%, at least 99.17%, at least 99.21%, at least 99.24%, at least 99.28%, at least 99.31%, at least 99.35%, at least 99.38%, at least 99.42%, at least 99.45%, at least 99.49%, at least 99.52%, at least 99.56%, at least 99.59%, at least 99.63%, at least 99.66%, at least 99.70%, at least 99.73%, at least 99.77%, at least 99.80%, at least 99.84%, at least 99.87%, at least 99.91%, at least 99.94%, at least 99.98%, or 100% identical) to SEQ ID NO: 67, and (e) a nucleic acid sequence that is at least 98.49% identical (e.g., at least 98.55%, at least 98.60%, at least 98.66%, at least 98.72%, at least 98.78%, at least 98.83%, at least 98.89%, at least 98.95%, at least 99.01%, at least 99.06%, at least 99.12%, at least 99.18%, at least 99.24%, at least 99.29%, at least 99.35%, at least 99.41%, at least 99.47%, at least 99.52%, at least 99.58%, at least 99.64%, at least 99.70%, at least 99.75%, at least 99.81%, at least 99.87%, at least 99.93%, at least 99.98%, or 100% identical) to SEQ ID NO: 68.

The adenovirus or adenoviral vector can comprise one, two, three, four, or all five of the aforementioned sequences alone or in any combination. In this respect, the adenovirus or adenoviral vector can comprise any combination of any two of the aforementioned sequences, any combination of any three of the aforementioned sequences, any combination of any four of the aforementioned sequences, or all five of the aforementioned sequences. For example, the adenovirus or adenoviral vector can comprise a nucleic acid sequence that is at least 98.4% identical to SEQ ID NO: 64. The adenovirus or adenoviral vector can comprise a nucleic acid sequence that is at least 99.01% identical to SEQ ID NO: 65 and a nucleic acid sequence that is at least 97.08% identical to SEQ ID NO: 66. The adenovirus or adenoviral vector can comprise a nucleic acid sequence that is at least 97.08% identical to SEQ ID NO: 66, a nucleic acid sequence that is at least 96.52% identical to SEQ ID NO: 67, and a nucleic acid sequence that is at least 98.49% identical to SEQ ID NO: 68. The adenovirus or adenoviral vector can comprise (a) the nucleic acid sequence of SEQ ID NO: 64, (b) the nucleic acid sequence SEQ ID NO: 65, (c) the nucleic acid sequence of SEQ ID NO: 66, (d) the nucleic acid sequence of SEQ ID NO: 67, or (e) the nucleic acid sequence of SEQ ID NO: 68. The adenovirus or adenoviral vector can comprise (a) a nucleic acid sequence that is at least 98.4% identical to SEQ ID NO: 64, (b) a nucleic acid sequence that is at least 99.01% identical to SEQ ID NO: 65, (c) a nucleic acid sequence that is at least 97.08% identical to SEQ ID NO: 66, (d) a nucleic acid sequence that is at least 96.52% identical to SEQ ID NO: 67, and (e) a nucleic acid sequence that is at least 98.49% identical to SEQ ID NO: 68. The adenovirus or adenoviral vector can comprise (a) the nucleic acid sequence of SEQ ID NO: 64, (b) the nucleic acid sequence SEQ ID NO: 65, (c) the nucleic acid sequence of SEQ ID NO: 66, (d) the nucleic acid sequence of SEQ ID NO: 67, and (e) the nucleic acid sequence of SEQ ID NO: 68.

In another embodiment, the adenovirus or adenoviral vector comprises one or more of the following nucleic acid sequences: (a) a nucleic acid sequence comprising at least 121 contiguous nucleotides of SEQ ID NO: 64, (b) a nucleic acid sequence comprising at least 462 contiguous nucleotides of SEQ ID NO: 65, (c) a nucleic acid sequence comprising at least 234 contiguous nucleotides of SEQ ID NO: 66, (d) a nucleic acid sequence comprising at least 606 contiguous nucleotides of SEQ ID NO: 67, or (e) a nucleic acid sequence comprising at least 188 contiguous nucleotides of SEQ ID NO: 68.

The adenovirus or adenoviral vector can comprise a nucleic acid sequence comprising at least 121 (e.g., 125 or more, 130 or more, 150 or more, 200 or more, 250 or more, or 300 or more) contiguous nucleotides of SEQ ID NO: 64, but no more than 399 (e.g., 398 or less, 350 or less, or 275 or less) contiguous nucleotides of SEQ ID NO: 64. Preferably, the adenovirus or adenoviral vector comprises a nucleic acid sequence comprising 121 to 300 contiguous nucleotides (e.g., 125, 150, 175, 200, 250, or 275 contiguous nucleotides), 121 to 200 contiguous nucleotides (e.g., 130, 140, 145, 160, 165, 170, 180, 185, 190, 195, or 199 contiguous nucleotides), or 121 to 150 contiguous nucleotides (e.g., 122, 123, 124, 125, 126, 127, 128, 129, 130, 131, 132, 133, 134, 135, 136, 137, 138, 139, 140, 141, 142, 143, 144, 145, 146, 147, 148, or 149 contiguous nucleotides) of SEQ ID NO: 64, or a range defined by any two of the foregoing values.

The adenovirus or adenoviral vector can comprise a nucleic acid sequence comprising at least 462 (e.g., 470 or more, 500 or more, 600 or more, 700 or more, 800 or more, 900 or more, or 1,000 or more) contiguous nucleotides of SEQ ID NO: 65, but no more than 3168 (e.g., 3,100 or less, 3,000 or less, 2,500 or less, 2,000 or less, or 1,500 or less) contiguous nucleotides of SEQ ID NO: 65. Preferably, the adenovirus or adenoviral vector comprises a nucleic acid sequence comprising 462 to 2,000 contiguous nucleotides (e.g., 475, 500, 700, 1,000, 1,200, 1,500, or 1,700 contiguous nucleotides), 462 to 1,000 contiguous nucleotides (e.g., 490, 525, 575, 600, 650, 675, 725, 750, 800, 850, 900, or 950 contiguous nucleotides), or 462 to 800 contiguous nucleotides (e.g., 480, 485, 490, 495, 499, 510, 515, 530, 540, 550, 560, 565, 570, 580, 585, 590, 595, 615, 625, 630, 640, 660, 665, 670, 680, 685, 690, 695, 705, 715, 730, 740, 755, 760, 765, 770, 775, 780, 785, 790, 795, or 799 contiguous nucleotides) of SEQ ID NO: 65, or a range defined by any two of the foregoing values.

The adenovirus or adenoviral vector can comprise a nucleic acid sequence comprising at least 234 (e.g., 235 or more, 250 or more, 300 or more, 350 or more, 400 or more, 450 or more, or 500 or more) contiguous nucleotides of SEQ ID NO: 66, but no more than 1,974 (e.g., 1,900 or less, 1,800 or less, 1,500 or less, 1,200 or less, 1,000 or less, 850 or less, 800 or less, 750 or less, or 700 or less) contiguous nucleotides of SEQ ID NO: 66. Preferably, the adenovirus or adenoviral vector comprises a nucleic acid sequence comprising 234 to 1,500 contiguous nucleotides (e.g., 290, 300, 400, 500, 600, 700, 800, 900, 1,000, or 1,200 contiguous nucleotides), 234 to 1,000 contiguous nucleotides (e.g., 295, 350, 450, 550, 650, 750, 850, or 950 contiguous nucleotides), or 234 to 500 contiguous nucleotides (e.g., 290, 305, 310, 315, 325, 340, 345, 360, 365, 370, 375, 380, 385, 390, 395, 405, 425, 430, 440, 455, 460, 465, 470, 475, 480, 485, 490, 495, or 499 contiguous nucleotides) of SEQ ID NO: 66, or a range defined by any two of the foregoing values.

The adenovirus or adenoviral vector can comprise a nucleic acid sequence comprising at least 606 (e.g., 610 or more, 650 or more, 700 or more, 800 or more, or 1,000 or more) contiguous nucleotides of SEQ ID NO: 67, but no more than 2865 (2,800 or less, 2,500 or less, 2,000 or less, 1,800 or less, or 1,500 or less) contiguous nucleotides of SEQ ID NO: 67. Preferably, the adenovirus or adenoviral vector comprises a nucleic acid sequence comprising 606 to 2,000 contiguous nucleotides (e.g., 615, 650, 700, 800, 900, 1,000, 1,200, 1,500, 1,700, or 1,900 contiguous nucleotides), 606 to 1,000 contiguous nucleotides (e.g., 630, 645, 665, 675, 725, 750, 775, 825, 850, 875, 925, 950, or 975 contiguous nucleotides), or 606 to 800 contiguous nucleotides (e.g., 620, 635, 640, 655, 660, 670, 680, 685, 690, 695, 699, 705, 715, 730, 735, 740, 745, 755, 760, 765, 770, 785, 790, 795, or 799 contiguous nucleotides) of SEQ ID NO: 67, or a range defined by any two of the foregoing values.

The adenovirus or adenoviral vector can comprise a nucleic acid sequence comprising at least 188 (e.g., 189 or more, 200 or more, 300 or more, 500 or more, 700 or more, or 900 or more) contiguous nucleotides of SEQ ID NO: 68, but no more than 1,740 (1,700 or less, 1,500 or less, 1,200 or less, or 1,000 or less) contiguous nucleotides of SEQ ID NO: 68. Preferably, the adenovirus or adenoviral vector comprises a nucleic acid sequence comprising 188 to 1,500 contiguous nucleotides (e.g., 200, 400, 600, 800, 1,000, 1,200, or 1,400 contiguous nucleotides), 188 to 1,000 contiguous nucleotides (e.g., 195, 250, 350, 450, 550, 650, 750, 850, or 950 contiguous nucleotides), or 188 to 500 contiguous nucleotides (e.g., 190, 225, 230, 240, 255, 260, 265, 270, 275, 315, 325, 330, 340, 355, 360, 365, 370, 375, 380, 385, 390, 395, 415, 425, 430, 440, 455, 460, 465, 470, 475, 480, 485, 490, 495, or 499 contiguous nucleotides) of SEQ ID NO: 68, or a range defined by any two of the foregoing values.

The adenovirus or adenoviral vector can comprise one, two, three, four, or all five of the aforementioned sequences alone, or in any combination. In this respect, the adenovirus or adenoviral vector can comprise any combination of any two of the aforementioned sequences, any combination of any three of the aforementioned sequences, any combination of any four of the aforementioned sequences, or all five of the aforementioned sequences. For example, the adenovirus or adenoviral vector can comprise a nucleic acid sequence comprising at least 121 contiguous nucleotides of SEQ ID NO: 64. The adenovirus or adenoviral vector can comprise a nucleic acid sequence comprising at least 234 contiguous nucleotides of SEQ ID NO: 66, and a nucleic acid sequence comprising at least 188 contiguous nucleotides of SEQ ID NO: 68. The adenovirus or adenoviral vector can comprise a nucleic acid sequence comprising at least 462 contiguous nucleotides of SEQ ID NO: 65, a nucleic acid sequence comprising at least 606 contiguous nucleotides of SEQ ID NO: 67, and a nucleic acid sequence comprising at least 188 contiguous nucleotides of SEQ ID NO: 68. The adenovirus or adenoviral vector can comprise a nucleic acid sequence comprising at least 121 contiguous nucleotides of SEQ ID NO: 64, a nucleic acid sequence comprising at least 462 contiguous nucleotides of SEQ ID NO: 65, a nucleic acid sequence comprising at least 234 contiguous nucleotides of SEQ ID NO: 66, and a nucleic acid sequence comprising at least 606 contiguous nucleotides of SEQ ID NO: 67. The adenovirus or adenoviral vector can comprise (a) a nucleic acid sequence comprising at least 121 contiguous nucleotides of SEQ ID NO: 64, (b) a nucleic acid sequence comprising at least 462 contiguous nucleotides of SEQ ID NO: 65, (c) a nucleic acid sequence comprising at least 234 contiguous nucleotides of SEQ ID NO: 66, (d) a nucleic acid sequence comprising at least 606 contiguous nucleotides of SEQ ID NO: 67, and (e) a nucleic acid sequence comprising at least 188 contiguous nucleotides of SEQ ID NO: 68.

In another embodiment, the adenovirus or adenoviral vector comprises one or more of the following amino acid sequences: (a) an amino acid sequence that is at least 93% identical (e.g., at least 96.57% or 100% identical) to SEQ ID NO: 69, (b) an amino acid sequence that is at least 80% identical (e.g., at least 86.67%, at least 93.33%, or 100% identical) to SEQ ID NO: 71, (c) an amino acid sequence that is at least 92% identical (e.g., at least 97.56% or 100% identical) to SEQ ID NO: 72, and (d) an amino acid sequence that is at least 88% identical (e.g., at least 94.67% or 100% identical) to SEQ ID NO: 73.

The adenovirus or adenoviral vector can comprise one, two, three, or all four of the aforementioned amino acid sequences alone or in any combination. In this respect, the adenovirus or adenoviral vector can comprise any combination of any two of the aforementioned sequences, any combination of any three of the aforementioned sequences, or all four of the aforementioned sequences. For example, the adenovirus or adenoviral vector can comprise an amino acid sequence that is at least 93% identical to SEQ ID NO: 69. The adenovirus or adenoviral vector can comprise an amino acid sequence that is at least 93% identical to SEQ ID NO: 69, and an amino acid sequence that is at least 80% identical to SEQ ID NO: 71. The adenovirus or adenoviral vector can comprise an amino acid sequence that is at least 93% identical to SEQ ID NO: 69, an amino acid sequence that is at least 80% identical to SEQ ID NO: 71, and an amino acid sequence that is at least 88% identical to SEQ ID NO: 73. The adenovirus or adenoviral vector can comprise (a) the amino acid sequence of SEQ ID NO: 69, (b) the amino acid sequence of SEQ ID NO: 71, (c) the amino acid sequence of SEQ ID NO: 72, or (d) the amino acid sequence of SEQ ID NO: 73. The adenovirus or adenoviral vector can comprise (a) an amino acid sequence that is at least 93% identical to SEQ ID NO: 69, (b) an amino acid sequence that is at least 80% identical to SEQ ID NO: 71, (c) an amino acid sequence that is at least 92% identical to SEQ ID NO: 72, and (d) an amino acid sequence that is at least 88% identical to SEQ ID NO: 73. The adenovirus or adenoviral vector can comprise (a) the amino acid sequence of SEQ ID NO: 69, (b) the amino acid sequence of SEQ ID NO: 71, (c) the amino acid sequence of SEQ ID NO: 72, and (d) the amino acid sequence of SEQ ID NO: 73.

In another embodiment, the adenovirus or adenoviral vector comprises one or more of the following amino acid sequences: (a) an amino acid sequence that is at least 99% identical (e.g., at least 99.75% or 100% identical) to SEQ ID NO: 74, (b) an amino acid sequence that is at least 97.8% identical (e.g., at least 97.95%, at least 98.10%, at least 98.26%, at least 98.41%, at least 98.56%, at least 98.71%, at least 98.86%, at least 99.02%, at least 99.17%, at least 99.32%, at least 99.47%, at least 99.62%, at least 99.78%, at least 99.93%, or 100% identical) to SEQ ID NO: 76, (c) an amino acid sequence that is at least 99.1% identical (e.g., at least 99.20%, at least 99.31%, at least 99.41%, at least 99.52%, at least 99.62%, at least 99.73%, at least 99.83%, at least 99.94%, or 100% identical) to SEQ ID NO: 77, and (d) an amino acid sequence that is at least 99.2% identical (e.g., at least 99.37%, at least 99.54%, at least 99.72%, at least 99.89%, or 100% identical) to SEQ ID NO: 78.

The adenovirus or adenoviral vector can comprise one, two, three, or all four of the aforementioned amino acid sequences alone or in any combination. In this respect, the adenovirus or adenoviral vector can comprise any combination of any two of the aforementioned sequences, any combination of any three of the aforementioned sequences, or all four of the aforementioned sequences. For example, the adenovirus or adenoviral vector can comprise an amino acid sequence that is at least 99% identical to SEQ ID NO: 74. The adenovirus or adenoviral vector can comprise an amino acid sequence that is at least 99% identical to SEQ ID NO: 74, and an amino acid sequence that is at least 99.1% identical to SEQ ID NO: 77. The adenovirus or adenoviral vector can comprise an amino acid sequence that is at least 99% identical to SEQ ID NO: 74, an amino acid sequence that is at least 99.1% identical to SEQ ID NO: 77, and an amino acid sequence that is at least 99.2% identical to SEQ ID NO: 78. The adenovirus or adenoviral vector can comprise (a) the amino acid sequence of SEQ ID NO: 74, (b) the amino acid sequence of SEQ ID NO: 76, (c) the amino acid sequence of SEQ ID NO: 77, or (d) the amino acid sequence of SEQ ID NO: 78. The adenovirus or adenoviral vector can comprise (a) an amino acid sequence that is at least 99% identical to SEQ ID NO: 74, (b) an amino acid sequence that is at least 97.8% identical to SEQ ID NO: 76, (c) an amino acid that is at least 99.1% identical to SEQ ID NO: 77, and (d) an amino acid sequence that is at least 99.2% identical to SEQ ID NO: 78. The adenovirus or adenoviral vector can comprise (a) the amino acid sequence of SEQ ID NO: 74, (b) the amino acid sequence of SEQ ID NO: 76, (c) the amino acid sequence of SEQ ID NO: 77, and (d) the amino acid sequence of SEQ ID NO: 78.

In another embodiment, the adenovirus or adenoviral vector comprises one or more of the following amino acid sequences: (a) an amino acid sequence comprising at least 89 contiguous amino acid residues of SEQ ID NO: 74, (b) an amino acid sequence comprising at least 247 contiguous amino acid residues of SEQ ID NO: 76, (c) an amino acid sequence comprising at least 230 contiguous amino acid residues of SEQ ID NO: 77, and (d) an amino acid sequence comprising at least 231 contiguous amino acid residues of SEQ ID NO: 78.

The adenovirus or adenoviral vector can comprise an amino acid sequence comprising at least 89 (e.g., 90 or more, 100 or more, or 110 or more) contiguous amino acid residues of SEQ ID NO: 74, but no more than 133 (e.g., 130 or less, 125 or less, 120 or less, or 115 or less) contiguous amino acid residues of SEQ ID NO: 74. Preferably, the adenovirus or adenoviral vector comprises an amino acid sequence comprising 89 to 130 contiguous amino acid residues (e.g., 90, 100, 110, 115, 120, or 125 contiguous amino acid residues) of SEQ ID NO: 74, 89 to 115 contiguous amino acid residues of SEQ ID NO: 74 (e.g., 95, 110, or 112 contiguous amino acid residues), or 89 to 100 contiguous amino acid residues (e.g., 89, 90, 91, 92, 93, 94, 95, 96, 97, 98, or 99 contiguous amino acid residues) of SEQ ID NO: 74, or a range defined by any two of the foregoing values.

The adenovirus or adenoviral vector can comprise an amino acid sequence comprising at least 247 (e.g., 250 or more, 275 or more, 300 or more, or 400 or more) contiguous amino acid residues of SEQ ID NO: 76, but no more than 658 (e.g., 650 or less, 550 or less, or 450 or less) contiguous amino acid residues of SEQ ID NO: 76. Preferably, the adenovirus or adenoviral vector comprises an acid sequence comprising 247 to 600 contiguous amino acid residues (e.g., 255, 275, 300, 400, or 500 contiguous amino acid residues) of SEQ ID NO: 76, 247 to 500 contiguous amino acid residues of SEQ ID NO: 76 (e.g., 325, 350, 375, 425, 450, or 475 contiguous amino acid residues), or 247 to 400 contiguous amino acid residues (e.g., 265, 280, 285, 290, 295, 360, 365, 380, 385, 390, 395, or 399 contiguous amino acid residues) of SEQ ID NO: 76, or a range defined by any two of the foregoing values.

The adenovirus or adenoviral vector can comprise an amino acid sequence comprising at least 230 (e.g., 250 or more, 300 or more, 350 or more, 400 or more, or 500 or more) contiguous amino acid residues of SEQ ID NO: 77, but no more than 955 (e.g., 950 or less, 900 or less, 800 or less, 700 or less, or 600 or less) contiguous amino acid residues of SEQ ID NO: 77. Preferably, the adenovirus or adenoviral vector comprises an acid sequence comprising 230 to 800 contiguous amino acid residues (e.g., 240, 260, 270, 280, 290, 300, 350, 390, 400, 500, 600, or 750 contiguous amino acid residues) of SEQ ID NO: 77, 230 to 600 contiguous amino acid residues (e.g., 255, 265, 275, 285, 295, 305, 325, 335, 345, 355, 365, 375, 385, 395, 425, 445, 450, 465, 475, 525, 545, 550, 565 or 575 contiguous amino acid residues) of SEQ ID NO: 77, or 230 to 500 contiguous amino acid residues (e.g., 235, 245, 299, 320, 330, 340, 360, 370, 385, 389, 395, 399, 415, 435, 440, 460, 470, 480, or 499 contiguous amino acid residues) of SEQ ID NO: 77, or a range defined by any two of the foregoing values.

The adenovirus or adenoviral vector can comprise an amino acid sequence comprising at least 231 (e.g., 250 or more, 300 or more, or 350 or more) contiguous amino acid residues of SEQ ID NO: 78, but no more than 580 (e.g., 575 or less, 550 or less, 500 or less, 450 or less, or 400 or less) contiguous amino acid residues of SEQ ID NO: 78. Preferably, the adenovirus or adenoviral vector comprises an acid sequence comprising 231 to 500 contiguous amino acid residues (e.g., 245, 255, 275, 300, 350, 375, or 400 contiguous amino acid residues) of SEQ ID NO: 78, 231 to 400 contiguous amino acid residues (e.g., 235, 265, 280, 285, 295, 305, 315, 325, 335, 345, 355, 365, 375, 385, 395, or 399 contiguous amino acid residues) of SEQ ID NO: 78, or 231 to 300 contiguous amino acid residues (e.g., 240, 250, 260, 270, or 299 contiguous amino acid residues) of SEQ ID NO: 78, or a range defined by any two of the foregoing values.

The adenovirus or adenoviral vector can comprise one, two, three, or all four of the aforementioned amino acid sequences alone or in any combination. In this respect, the adenovirus or adenoviral vector can comprise any combination of any two of the aforementioned sequences, any combination of any three of the aforementioned sequences, or all four of the aforementioned sequences. For example, the adenovirus or adenoviral vector can comprise an amino acid sequence comprising at least 89 contiguous amino acid residues of SEQ ID NO: 74. The adenovirus or adenoviral vector can comprise an amino acid sequence comprising at least 89 contiguous amino acid residues of SEQ ID NO: 74, and an amino acid sequence comprising at least 230 contiguous amino acid residues of SEQ ID NO: 77. The adenovirus or adenoviral vector can comprise an amino acid sequence comprising at least 89 contiguous amino acid residues of SEQ ID NO: 74, an amino acid sequence comprising at least 247 contiguous amino acid residues of SEQ ID NO: 76, and an amino acid sequence comprising at least 230 contiguous amino acid residues of SEQ ID NO: 77. The adenovirus or adenoviral vector can comprise an amino acid sequence comprising at least 89 contiguous amino acid residues of SEQ ID NO: 74, an amino acid sequence comprising at least 247 contiguous amino acid residues of SEQ ID NO: 76, and an amino acid sequence comprising at least 231 contiguous amino acid residues of SEQ ID NO: 78. The adenovirus or adenoviral vector can comprise (a) an amino acid sequence comprising at least 89 contiguous amino acid residues of SEQ ID NO: 74, (b) an amino acid sequence comprising at least 247 contiguous amino acid residues of SEQ ID NO: 76, (c) an amino acid sequence comprising at least 230 contiguous amino acid residues of SEQ ID NO: 77, and (d) an amino acid sequence comprising at least 231 contiguous amino acid residues of SEQ ID NO: 78.

In other embodiments, the adenovirus or adenoviral vector comprises one or more nucleic acid sequences that encode one or more of any of the aforementioned amino acid sequences, e.g., the amino acid sequences of any of SEQ ID NOs: 69-78 or any of the variants and/or portions thereof as described herein. For example, the adenovirus or adenoviral vector can comprise a nucleic acid sequence encoding an amino acid sequence that is at least 99.5% identical (e.g., at least 99.59%, at least 99.69%, at least 99.78%, at least 99.88%, at least 99.97%, or 100% identical) to SEQ ID NO: 75, or a nucleic acid sequence encoding an amino acid sequence that is at least 95% identical (e.g., at least 97.94% or 100% identical) to SEQ ID NO: 70.

The adenovirus or adenoviral vector can comprise the nucleic acid sequence of, for example, SEQ ID NO: 21, SEQ ID NO: 22, SEQ ID NO: 23, SEQ ID NO: 24, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 79, SEQ ID NO: 80, SEQ ID NO: 81, or SEQ ID NO: 82.

As discussed herein, the adenovirus or adenoviral vector can be replication-competent, conditionally-replicating, or replication-deficient. Preferably, the adenovirus or adenoviral vector is replication-deficient, such that the replication-deficient adenovirus or adenoviral vector requires complementation of at least one replication-essential gene function of one or more regions of the adenoviral genome for propagation (e.g., to form adenoviral vector particles).

The replication-deficient adenovirus or adenoviral vector can be modified in any suitable manner to cause the deficiencies in the one or more replication-essential gene functions in one or more regions of the adenoviral genome for propagation. The complementation of the deficiencies in the one or more replication-essential gene functions of one or more regions of the adenoviral genome refers to the use of exogenous means to provide the deficient replication-essential gene functions. Such complementation can be effected in any suitable manner, for example, by using complementing cells and/or exogenous DNA (e.g., helper adenovirus) encoding the disrupted replication-essential gene functions.

The adenovirus or adenoviral vector can be deficient in one or more replication-essential gene functions of only the early regions (i.e., E1-E4 regions) of the adenoviral genome, only the late regions (i.e., L1-L5 regions) of the adenoviral genome, both the early and late regions of the adenoviral genome, or all adenoviral genes (i.e., a high capacity adenovector (HC-Ad). See Morsy et al., Proc. Natl. Acad. Sci. USA, 95: 965-976 (1998); Chen et al., Proc. Natl. Acad. Sci. USA, 94: 1645-1650 (1997); and Kochanek et al., Hum. Gene Ther., 10: 2451-2459 (1999). Examples of replication-deficient adenoviral vectors are disclosed in U.S. Pat. Nos. 5,837,511; 5,851,806; 5,994,106; 6,127,175; 6,482,616; and 7,195,896, and International Patent Application Publications WO 1994/028152, WO 1995/002697, WO 1995/016772, WO 1995/034671, WO 1996/022378, WO 1997/012986, WO 1997/021826, and WO 2003/022311.

The early regions of the adenoviral genome include the E1, E2, E3, and E4 regions. The E1 region comprises the E1A and E1B subregions, and one or more deficiencies in replication-essential gene functions in the E1 region can include one or more deficiencies in replication-essential gene functions in either or both of the E1A and E1B subregions, thereby requiring complementation of the E1A subregion and/or the E1B subregion of the adenoviral genome for the adenovirus or adenoviral vector to propagate (e.g., to form adenoviral vector particles). The E2 region comprises the E2A and E2B subregions, and one or more deficiencies in replication-essential gene functions in the E2 region can include one or more deficiencies in replication-essential gene functions in either or both of the E2A and E2B subregions, thereby requiring complementation of the E2A subregion and/or the E2B subregion of the adenoviral genome for the adenovirus or adenoviral vector to propagate (e.g., to form adenoviral vector particles).

The E3 region does not include any replication-essential gene functions, such that a deletion of the E3 region in part or in whole does not require complementation of any gene functions in the E3 region for the adenovirus or adenoviral vector to propagate (e.g., to form adenoviral vector particles). In the context of the invention, the E3 region is defined as the region that initiates with the open reading frame that encodes a protein with high homology to the 12.5K protein from the E3 region of human adenovirus 5 (NCBI reference sequence AP_{—}000218) and ends with the open reading frame that encodes a protein with high homology to the 14.7K protein from the E3 region of human adenovirus 5 (NCBI reference sequence AP_{—}000224.1). The E3 region may be deleted in whole or in part, or retained in whole or in part. The size of the deletion may be tailored so as to retain an adenovirus or adenoviral vector whose genome closely matches the optimum genome packaging size. A larger deletion will accommodate the insertion of larger heterologous nucleic acid sequences in the adenovirus or adenoviral genome. In one embodiment of the invention, the LA polyadenylation signal sequences, which reside in the E3 region, are retained.

The E4 region comprises multiple open reading frames (ORFs). An adenovirus or adenoviral vector with a deletion of all of the open reading frames of the E4 region except ORF6, and in some cases ORF3, does not require complementation of any gene functions in the E4 region for the adenovirus or adenoviral vector to propagate (e.g., to form adenoviral vector particles). Conversely, an adenovirus or adenoviral vector with a disruption or deletion of ORF6, and in some cases ORF3, of the E4 region (e.g., with a deficiency in a replication-essential gene function based in ORF6 and/or ORF3 of the E4 region), with or without a disruption or deletion of any of the other open reading frames of the E4 region or the native E4 promoter, polyadenylation sequence, and/or the right-side inverted terminal repeat (ITR), requires complementation of the E4 region (specifically, of ORF6 and/or ORF3 of the E4 region) for the adenovirus or adenoviral vector to propagate (e.g., to form adenoviral vector particles).

The late regions of the adenoviral genome include the L1, L2, L3, LA, and L5 regions. The adenovirus or adenoviral vector also can have a mutation in the major late promoter (MLP), as discussed in International Patent Application Publication WO 2000/000628, which can render the adenovirus or adenoviral vector replication-deficient if desired.

The one or more regions of the adenoviral genome that contain one or more deficiencies in replication-essential gene functions desirably are one or more early regions of the adenoviral genome, i.e., the E1, E2, and/or E4 regions, optionally with the deletion in part or in whole of the E3 region.

The replication-deficient adenovirus or adenoviral vector also can have one or more mutations as compared to the wild-type adenovirus (e.g., one or more deletions, insertions, and/or substitutions) in the adenoviral genome that do not inhibit viral replication in host cells. Thus, in addition to one or more deficiencies in replication-essential gene functions, the adenovirus or adenoviral vector can be deficient in other respects that are not replication-essential. For example, the adenovirus or adenoviral vector can have a partial or entire deletion of the adenoviral early region known as the E3 region, which is not essential for propagation of the adenovirus or adenoviral genome.

In one embodiment, the adenovirus or adenoviral vector is replication-deficient and requires, at most, complementation of the E1 region or the E4 region of the adenoviral genome, for propagation (e.g., to form adenoviral vector particles). Thus, the replication-deficient adenovirus or adenoviral vector requires complementation of at least one replication-essential gene function of the E1A subregion and/or the E1B region of the adenoviral genome (denoted an E1-deficient adenoviral vector) or the E4 region of the adenoviral genome (denoted an E4-deficient adenoviral vector) for propagation (e.g., to form adenoviral vector particles). The adenovirus or adenoviral vector can be deficient in at least one replication-essential gene function (desirably all replication-essential gene functions) of the E1 region of the adenoviral genome and at least one gene function of the nonessential E3 region of the adenoviral genome (denoted an E1/E3-deficient adenoviral vector). The adenovirus or adenoviral vector can be deficient in at least one replication-essential gene function (desirably all replication-essential gene functions) of the E4 region of the adenoviral genome and at least one gene function of the nonessential E3 region of the adenoviral genome (denoted an E3/E4-deficient adenoviral vector).

In one embodiment, the adenovirus or adenoviral vector is replication-deficient and requires, at most, complementation of the E2 region, preferably the E2A subregion, of the adenoviral genome, for propagation (e.g., to form adenoviral vector particles). Thus, the replication-deficient adenovirus or adenoviral vector requires complementation of at least one replication-essential gene function of the E2A subregion of the adenoviral genome (denoted an E2A-deficient adenoviral vector) for propagation (e.g., to form adenoviral vector particles). The adenovirus or adenoviral vector can be deficient in at least one replication-essential gene function (desirably all replication-essential gene functions) of the E2A region of the adenoviral genome and at least one gene function of the nonessential E3 region of the adenoviral genome (denoted an E2A/E3-deficient adenoviral vector).

In one embodiment, the adenovirus or adenoviral vector is replication-deficient and requires, at most, complementation of the E1 and E4 regions of the adenoviral genome for propagation (e.g., to form adenoviral vector particles). Thus, the replication-deficient adenovirus or adenoviral vector requires complementation of at least one replication-essential gene function of both the E1 and E4 regions of the adenoviral genome (denoted an E1/E4-deficient adenoviral vector) for propagation (e.g., to form adenoviral vector particles). The adenovirus or adenoviral vector can be deficient in at least one replication-essential gene function (desirably all replication-essential gene functions) of the E1 region of the adenoviral genome, at least one replication-essential gene function of the E4 region of the adenoviral genome, and at least one gene function of the nonessential E3 region of the adenoviral genome (denoted an E1/E3/E4-deficient adenoviral vector). The adenovirus or adenoviral vector preferably requires, at most, complementation of the E1 region of the adenoviral genome for propagation, and does not require complementation of any other deficiency of the adenoviral genome for propagation. More preferably, the adenovirus or adenoviral vector requires, at most, complementation of the E1 and E4 regions of the adenoviral genome for propagation, and does not require complementation of any other deficiency of the adenoviral genome for propagation.

The adenovirus or adenoviral vector, when deficient in multiple replication-essential gene functions of the adenoviral genome (e.g., an E1/E4-deficient adenoviral vector), can include a spacer sequence to provide viral growth in a complementing cell line similar to that achieved by adenoviruses or adenoviral vectors deficient in a single replication-essential gene function (e.g., an E1-deficient adenoviral vector). The spacer sequence can contain any nucleotide sequence or sequences which are of a desired length, such as sequences at least about 15 base pairs (e.g., between about 15 nucleotides and about 12,000 nucleotides), preferably about 100 nucleotides to about 10,000 nucleotides, more preferably about 500 nucleotides to about 8,000 nucleotides, even more preferably about 1,500 nucleotides to about 6,000 nucleotides, and most preferably about 2,000 to about 3,000 nucleotides in length, or a range defined by any two of the foregoing values. The spacer sequence can be coding or non-coding and native or non-native with respect to the adenoviral genome, but does not restore the replication-essential function to the deficient region. The spacer also can contain an expression cassette. More preferably, the spacer comprises a polyadenylation sequence and/or a gene that is non-native with respect to the adenovirus or adenoviral vector. The use of a spacer in an adenoviral vector is further described in, for example, U.S. Pat. No. 5,851,806 and International Patent Application Publication WO 1997/021826.

By removing all or part of the adenoviral genome, for example, the E1, E3, and E4 regions of the adenoviral genome, the resulting adenovirus or adenoviral vector is able to accept inserts of exogenous nucleic acid sequences while retaining the ability to be packaged into adenoviral capsids. An exogenous nucleic acid sequence can be inserted at any position in the adenoviral genome so long as insertion in the position allows for the formation of adenovirus or the adenoviral vector particle. The exogenous nucleic acid sequence preferably is positioned in the E1 region, the E3 region, or the E4 region of the adenoviral genome.

The replication-deficient adenovirus or adenoviral vector of the invention can be produced in complementing cell lines that provide gene functions not present in the replication-deficient adenovirus or adenoviral vector, but required for viral propagation, at appropriate levels in order to generate high titers of viral vector stock. Such complementing cell lines are known and include, but are not limited to, 293 cells (described in, e.g., Graham et al., J. Gen. Virol., 36: 59-72 (1977)), PER.C6 cells (described in, e.g., International Patent Application Publication WO 1997/000326, and U.S. Pat. Nos. 5,994,128 and 6,033,908), and 293-ORF6 cells (described in, e.g., International Patent Application Publication WO 1995/034671 and Brough et al., J. Virol., 71: 9206-9213 (1997)). Other suitable complementing cell lines to produce the replication-deficient adenovirus or adenoviral vector of the invention include complementing cells that have been generated to propagate adenoviral vectors encoding transgenes whose expression inhibits viral growth in host cells (see, e.g., U.S. Patent Application Publication 2008/0233650). Additional suitable complementing cells are described in, for example, U.S. Pat. Nos. 6,677,156 and 6,682,929, and International Patent Application Publication WO 2003/020879. In some instances, the cellular genome need not comprise nucleic acid sequences, the gene products of which complement for all of the deficiencies of a replication-deficient adenoviral vector. One or more replication-essential gene functions lacking in a replication-deficient adenoviral vector can be supplied by a helper virus, e.g., an adenoviral vector that supplies in trans one or more essential gene functions required for replication of the replication-deficient adenovirus or adenoviral vector. Alternatively, the inventive adenovirus or adenoviral vector can comprise a non-native replication-essential gene that complements for the one or more replication-essential gene functions lacking in the inventive replication-deficient adenovirus or adenoviral vector. For example, an E1/E4-deficient adenoviral vector can be engineered to contain a nucleic acid sequence encoding E4 ORF 6 that is obtained or derived from a different adenovirus (e.g., an adenovirus of a different serotype than the inventive adenovirus or adenoviral vector, or an adenovirus of a different species than the inventive adenovirus or adenoviral vector).

The adenovirus or adenoviral vector can further comprise a transgene. The term “transgene” is defined herein as a non-native nucleic acid sequence that is operably linked to appropriate regulatory elements (e.g., a promoter), such that the non-native nucleic acid sequence can be expressed to produce a protein (e.g., peptide or polypeptide). The regulatory elements (e.g., promoter) can be native or non-native to the adenovirus or adenoviral vector.

A “non-native” nucleic acid sequence is any nucleic acid sequence (e.g., DNA, RNA, or cDNA sequence) that is not a naturally occurring nucleic acid sequence of an adenovirus in a naturally occurring position. Thus, the non-native nucleic acid sequence can be naturally found in an adenovirus, but located at a non-native position within the adenoviral genome and/or operably linked to a non-native promoter. The terms “non-native nucleic acid sequence,” “heterologous nucleic acid sequence,” and “exogenous nucleic acid sequence” are synonymous and can be used interchangeably in the context of the invention. The non-native nucleic acid sequence preferably is DNA and preferably encodes a protein (i.e., one or more nucleic acid sequences encoding one or more proteins).

The adenovirus or adenoviral vector preferably comprises at least one non-native nucleic acid that encodes an antigen of Plasmodium. An “antigen” is a molecule that triggers an immune response in a mammal. An “immune response” can entail, for example, antibody production and/or the activation of immune effector cells. An antigen in the context of the invention can comprise any subunit, fragment, or epitope of any proteinaceous or non-proteinaceous (e.g., carbohydrate or lipid) molecule which provokes an immune response in a mammal. By “epitope” is meant a sequence of an antigen that is recognized by an antibody or an antigen receptor. Epitopes also are referred to in the art as “antigenic determinants.”

A Plasmodium antigen in the context of the invention can comprise any proteinaceous Plasmodium molecule or portion thereof that provokes a Plasmodium-related immune response in a mammal. A “Plasmodium molecule” is a molecule that is a part of a Plasmodium parasite, is encoded by a nucleic acid sequence of a Plasmodium parasite, or is derived from or synthetically based upon any such molecule. Administration of a Plasmodium antigen that provokes an immune response in accordance with the invention preferably leads to protective immunity against Plasmodium. In this regard, an “immune response” to Plasmodium is an immune response to any one or more Plasmodium antigens.

The antigen can be from any suitable Plasmodium species, but preferably is from a Plasmodium species that infects humans and causes malaria. Human-infecting Plasmodium species include P. malariae, P. ovale, P. vivax, and P. falciparum. P. vivax and P. falciparum are the most common, and P. falciparum is the most deadly, species of Plasmodium in human. Alternatively, the antigen can be from a species of Plasmodium that infects non-human animals. For example, P. vinckei, P. chabaudi, P. yoelii, and P. berghei infect rodents. P. knowlesi, P. cynomolgi, P. simiovale, P. fieldi, P. inui, and P. brasilianum infect non-human primates. P. gallinaceum infects birds. In order to advance vaccine discovery, the genomes of a number of Plasmodium species have been sequenced. For example, the complete P. falciparum genome has been sequenced and is disclosed in Gardner et al., Nature, 419: 498-511 (2002). In addition, the complete P. yoelii genome sequence is disclosed in Carlton et al., Nature, 419: 512-9 (2002). Thus, one of ordinary skill in the art can identify and isolate an appropriate Plasmodium antigen using routine methods known in the art.

The heterologous nucleic acid sequence can encode any suitable P. falciparum antigen, but preferably encodes an antigen that is expressed during the blood-stage of infection (a “blood-stage antigen”) and/or an antigen that is expressed during the pre-erythrocytic stage of infection (a “pre-erythrocytic stage antigen”). Blood-stage antigens and pre-erythrocytic stage antigens activate the humoral (i.e., antibody-mediated) and cell-mediated (i.e., T cell) arms of the immune system. Suitable pre-erythrocytic stage antigens include, but are not limited to, circumsporozoite protein (CSP), sporozoite surface protein 2 (SSP2) (also known as thrombospondin-related anonymous protein (TRAP)), liver-stage antigen 1 (LSA-1), Pf exported protein 1 (P/Exp-1), and Pf Antigen 2 (also known as CelTos). Preferred pre-erythrocytic stage antigens include CSP, SSP2, and CelTos. Nucleic acid sequences encoding P. falciparum CSP are disclosed in, e.g., Jongwutiwes et al., Am. J. Trop. Med. Hyg., 51(5): 659-668 (1994), Dame et al., Science, 225: 593-599 (1984), and del Portillo et al., Mol. Biochem., Parasitol., 24(3): 289-294 (1987), and are publicly available (see, e.g., GenBank Accession Nos. M83161.1, M83156.1, M83152.1, M83149.1, M83173.1, M83170.1, M83168.1, M83166.1, M83164.1, and M60972.1). Nucleic acid sequences encoding P. falciparum SSP2 protein are publicly available (see, e.g., GenBank Accession Nos. AF249739.1 and XM_{—}001350052). Nucleic acid sequences encoding P. falciparum CelTos protein are publicly available (see, e.g., GenBank Accession No. XM_{—}001350533.1).

Suitable blood-stage antigens include, but are not limited to, merozoite surface protein 1 (MSP-1), merozoite surface protein 2 (MSP-2), erythrocyte binding antigen 175 (EBA-175), ring-infected erythrocyte surface antigen (RESA), serine repeat antigen (SERA), glycophorin binding protein (GBP-130), histidine rich protein 2 (HRP-2), rhoptry-associated proteins 1 and 2 (RAP-1 and RAP-2), erythrocyte membrane protein I (PEMP 1), and apical membrane antigen 1 (AMA-1). Preferably, the blood-stage antigen is AMA-1. Nucleic acid sequences encoding P. falciparum AMA-1 protein are publicly available (see, e.g., GenBank Accession No. AF277003.1)

In a preferred embodiment, the Plasmodium-encoding nucleic acid sequence comprises codons expressed more frequently (and preferably, most frequently) in humans than in Plasmodium. While the genetic code is generally universal across species, the choice among synonymous codons is often species-dependent. One of ordinary skill in the art would appreciate that, to achieve maximum protection against Plasmodium infection, the adenovirus or adenoviral vector must be capable of expressing high levels of Plasmodium antigens in a mammalian, preferably a human, host. In this respect, the nucleic acid sequence preferably encodes the native amino acid sequence of a Plasmodium antigen, but comprises codons that are expressed more frequently in mammals (e.g., humans) than in Plasmodium, and lacks cis signals that are inhibitory to high level expression of the Plasmodium antigen. Changing native Plasmodium codons to the most frequently used in mammals will increase expression of the Plasmodium antigen in a mammal (e.g., a human). Such modified nucleic acid sequences are commonly described in the art as “humanized,” as “codon-optimized,” or as utilizing “mammalian-preferred” or “human-preferred” codons.

In the context of the invention, a Plasmodium nucleic acid sequence is said to be “codon-optimized” if at least about 60% (e.g., at least about 70%, at least about 80%, or at least about 90%) of the wild-type codons in the nucleic acid sequence are modified to encode mammalian-preferred codons. That is, a Plasmodium nucleic acid sequence is codon-optimized if at least about 60% of the codons in the nucleic acid sequence are mammalian-preferred codons. Additionally and alternatively, the codon-optimized nucleic acid sequence encoding a Plasmodium antigen can be any sequence that hybridizes to an above-described sequence under at least moderate, preferably high, stringency conditions, such as described in, e.g., Sambrook et al., Molecular Cloning, a Laboratory Manual, 3rd edition, Cold Spring Harbor Press, Cold Spring Harbor, N.Y. (2001). Determining the degree of homology can be accomplished using any suitable method known in the art, such as those described herein.

In addition to the nucleic acid encoding a Plasmodium antigen, the adenovirus or adenoviral vector preferably comprises expression control sequences, such as promoters, enhancers, polyadenylation signals, transcription terminators, internal ribosome entry sites (IRES), and the like, that provide for the expression of the nucleic acid sequence in a host cell. Exemplary expression control sequences are known in the art and are described in, for example, Goeddel, Gene Expression Technology: Methods in Enzymology, Vol. 185, Academic Press, San Diego, Calif. (1990). Ideally, the Plasmodium antigen-encoding nucleic acid sequence is operably linked to a promoter and a polyadenylation sequence. The promoter desirably is a constitutive or inducible promoter, preferably a constitutive promoter, A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter (human or mouse), the β-actin promoter (human or chicken), the EF1-α promoter, the ubiquitin promoter, the SV40 promoter, and the Rous Sarcoma Virus promoter. Inducible promoters include, for example, the Tet system (U.S. Pat. Nos. 5,464,758 and 5,814,618), the Ecdysone inducible system (No et al., Proc. Natl. Acad. Sci., 93: 3346-3351 (1996)), the T-REx™ system (Invitrogen, Carlsbad, Calif.), LACSWITCH™ system (Stratagene, San Diego, Calif.), and the Cre-ERT tamoxifen inducible recombinase system (Indra et al., Nuc. Acid. Res., 27: 4324-4327 (1999); Nuc. Acid. Res., 28: e99 (2000); U.S. Pat. No. 7,112,715; and Kramer & Fussenegger, Methods Mol. Biol., 308: 123-144 (2005)).

A promoter can be selected by matching its particular pattern of activity with the desired pattern and level of expression of an antigen(s). For example, the adenovirus or adenoviral vector can comprise two or more nucleic acid sequences that encode the same or different antigens and are operably linked to different promoters displaying distinct expression profiles. In this regard, a first promoter can be selected to mediate an initial peak of antigen production, thereby priming the immune system against an encoded antigen. A second promoter can be selected to drive production of the same or different antigen such that expression peaks several days after that of the first promoter, thereby “boosting” the immune system against the antigen. Alternatively, a hybrid promoter can be constructed which combines the desirable aspects of multiple promoters. For example, a CMV-Rous Sarcoma Virus hybrid promoter combining the CMV promoter's initial rush of activity with the Rous Sarcoma Virus promoter's high maintenance level of activity can be employed. In that antigens can be toxic to eukaryotic cells, it may be advantageous to modify the promoter to decrease activity in complementing cell lines used to propagate the adenovirus or adenoviral vector.

To optimize protein production, preferably the antigen-encoding nucleic acid sequence further comprises a polyadenylation site following the coding sequence. Any suitable polyadenylation sequence can be used, including a synthetic optimized sequence, as well as the polyadenylation sequence of BGH (Bovine Growth Hormone), polyoma virus, TK (Thymidine Kinase), EBV (Epstein Barr Virus), mouse globin D protein, and the papillomaviruses, including human papillomaviruses and BPV (Bovine Papilloma Virus). A preferred polyadenylation sequence is the SV40 (Human Sarcoma Virus-40) polyadenylation sequence. Also, preferably all the proper transcription signals (and translation signals, where appropriate) are correctly arranged such that the nucleic acid sequence is properly expressed in the cells into which it is introduced. If desired, the nucleic acid sequence also can incorporate splice sites (i.e., splice acceptor and splice donor sites) to facilitate mRNA production.

If the Plasmodium antigen-encoding nucleic acid sequence encodes a processed or secreted protein or peptide, or a protein that acts intracellularly, preferably the Plasmodium antigen-encoding nucleic acid sequence further comprises the appropriate sequences for processing, secretion, intracellular localization, and the like. The Plasmodium antigen-encoding nucleic acid sequence can be operably linked to a signal sequence, which targets a protein to cellular machinery for secretion. Appropriate signal sequences include, but are not limited to, leader sequences for immunoglobulin heavy chains and cytokines (see, for example, Ladunga et al., Current Opinions in Biotechnology, 11: 13-18 (2000)). Other protein modifications can be required to secrete a protein from a host cell, which can be determined using routine laboratory techniques. Preparing expression constructs encoding antigens and signal sequences is further described in, for example, U.S. Pat. No. 6,500,641. Methods of secreting non-secretable proteins are further described in, for example, U.S. Pat. No. 6,472,176, and International Patent Application Publication WO 2002/048377.

A Plasmodium antigen encoded by the nucleic acid sequence of the adenovirus or adenoviral vector also can be modified to attach or incorporate the antigen on a host cell surface. In this respect, the antigen can comprise a membrane anchor, such as a gpi-anchor, for conjugation onto a cell surface. A transmembrane domain can be fused to the antigen to incorporate a terminus of the antigen protein into the cell membrane. Other strategies for displaying peptides on a cell surface are known in the art and are appropriate for use in the context of the invention.

The invention provides a composition comprising the adenovirus or adenoviral vector described herein and a carrier therefor (e.g., a pharmaceutically acceptable carrier). The composition desirably is a physiologically acceptable (e.g., pharmaceutically acceptable) composition, which comprises a carrier, preferably a physiologically (e.g., pharmaceutically) acceptable carrier, and the adenovirus or adenoviral vector. Any suitable carrier can be used within the context of the invention, and such carriers are well known in the art. The choice of carrier will be determined, in part, by the particular use of the composition (e.g., administration to an animal) and the particular method used to administer the composition. Ideally, in the context of replication-deficient adenoviral vectors, the pharmaceutical composition preferably is free of replication-competent adenovirus. The pharmaceutical composition optionally can be sterile.

Suitable compositions include aqueous and non-aqueous isotonic sterile solutions, which can contain anti-oxidants, buffers, and bacteriostats, and aqueous and non-aqueous sterile suspensions that can include suspending agents, solubilizers, thickening agents, stabilizers, and preservatives. The composition can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets. Preferably, the carrier is a buffered saline solution. More preferably, the adenovirus or adenoviral vector is part of a composition formulated to protect the adenovirus or adenoviral vector from damage prior to administration. For example, the composition can be formulated to reduce loss of the adenovirus or adenoviral vector on devices used to prepare, store, or administer the adenovirus or adenoviral vector, such as glassware, syringes, or needles. The composition can be formulated to decrease the light sensitivity and/or temperature sensitivity of the adenovirus or adenoviral vector. To this end, the composition preferably comprises a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a composition will extend the shelf life of the adenovirus or adenoviral vector, and facilitate its administration. Formulations for adenovirus or adenoviral vector-containing compositions are further described in, for example, U.S. Pat. No. 6,225,289, U.S. Pat. No. 6,514,943, and International Patent Application Publication WO 2000/034444.

The composition also can be formulated to enhance transduction efficiency. In addition, one of ordinary skill in the art will appreciate that the adenovirus or adenoviral vector can be present in a composition with other therapeutic or biologically-active agents. For example, factors that control inflammation, such as ibuprofen or steroids, can be part of the composition to reduce swelling and inflammation associated with in vivo administration of the adenovirus or adenoviral vector. Antibiotics, i.e., microbicides and fungicides, can be present to treat existing infection and/or reduce the risk of future infection, such as infection associated with gene transfer procedures.

The invention further provides a method of inducing an immune response against Plasmodium falciparum in a mammal. The method comprises administering to the mammal the inventive adenovirus or adenoviral vector alone, preferably the inventive composition comprising the adenovirus or adenoviral vector and a pharmaceutically acceptable carrier, whereupon the nucleic acid sequence encoding the Plasmodium antigen is expressed in the mammal to produce the Plasmodium antigen and thereby induce an immune response against Plasmodium falciparum in the mammal. In accordance with the invention, the composition is administered to a mammal, most preferably a human, wherein the Plasmodium antigen-encoding nucleic acid sequence is expressed to induce an immune response against Plasmodium falciparum in the mammal. The human preferably is in a population that has a high risk of acquiring Plasmodium falciparum. Such high-risk populations include residents of parts of Africa, Asia, the Middle East, Central and South America, Hispaniola, and Oceania, as well as military personnel deployed to these areas.

The immune response can be a humoral immune response, a cell-mediated immune response, or, desirably, a combination of humoral and cell-mediated immunity. Ideally, the immune response provides protection to the animal, typically a mammal such as a human, upon subsequent challenge with Plasmodium. However, protective immunity is not required in the context of the invention. The inventive method also can be used for antibody production and harvesting.

To enhance the immune response generated against a Plasmodium antigen, the composition also can comprise an immune stimulator, or a nucleic acid sequence that encodes an immune stimulator. Immune stimulators also are referred to in the art as “adjuvants,” and include, for example, cytokines, chemokines, or chaperones. Cytokines include, for example, Macrophage Colony Stimulating Factor (e.g., GM-CSF), Interferon Alpha (IFN-α), Interferon Beta (IFN-β), Interferon Gamma (IFN-γ), interleukins (IL-1, IL-2, IL-4, IL-5, IL-6, IL-8, IL-10, IL-12, IL-13, IL-15, IL-16, and IL-18), the TNF family of proteins, Intercellular Adhesion Molecule-1 (ICAM-1), Lymphocyte Function-Associated antigen-3 (LFA-3), B7-1, B7-2, FMS-related tyrosine kinase 3 ligand, (Flt3L), vasoactive intestinal peptide (VIP), and CD40 ligand. Chemokines include, for example, B Cell-Attracting chemokine-1 (BCA-1), Fractalkine, Melanoma Growth Stimulatory Activity (MGSA) protein, Hemofiltrate CC chemokine 1 (HCC-1), Interleukin 8 (IL-8), Interferon-stimulated T-cell alpha chemoattractant (I-TAC), Lymphotactin, Monocyte Chemotactic Protein I (MCP-1), Monocyte Chemotactic Protein 3 (MCP-3), Monocyte Chemotactic Protein 4 (CP-4), Macrophage-Derived Chemokine (MDC), a macrophage inflammatory protein (MIP), Platelet Factor 4 (PF4), RANTES, BRAK, eotaxin, exodus 1-3, and the like. Chaperones include, for example, the heat shock proteins Hsp170, Hsc70, and Hsp40.

The composition ideally comprises an “effective amount” of adenoviral vector, i.e., a dose of adenoviral vector which provokes a desired immune response in the mammal. Desirably, a single dose of adenoviral vector comprises about 1×10⁵ or more particles (which also are referred to as particle units (pu)) of the adenoviral vector, e.g., about 1×10⁶ or more particles, about 1×10⁷ or more particles, about 1×10⁸ or more particles, about 1×10⁹ or more particles, or about 3×10⁸ or more particles of the adenoviral vector. Alternatively, or in addition, a single dose of adenoviral vector comprises about 3×10¹⁴ particles or less of the adenoviral vector, e.g., about 1×10¹³ particles or less, about 1×10¹² particles or less, about 3×10¹¹ particles or less, about 1×10¹¹ particles or less, about 1×10¹⁰ particles or less, or about 1×10⁹ particles or less of the adenoviral vector. Thus, a single dose of adenoviral vector can comprise a quantity of particles of the adenoviral vector in a range defined by any two of the aforementioned values. For example, a single dose of adenoviral vector can comprise 1×10⁵-1×10⁴ particles, 1×107-1×10¹² particles, 1×10⁸-1×10¹¹ particles, 3×10⁸-3×10¹¹ particles, 1×10⁹-1×10¹² particles, 1×10⁹-1×10¹¹ particles, 1×10⁹-1×10¹⁰ particles, or 1×10¹⁰-1×10¹² particles, of the adenoviral vector. In other words, a single dose of adenoviral vector can comprise, for example, about 1×10⁶ pu, 2×10⁶ pu, 4×10⁶ pu, 1×10⁷ pu, 2×10⁷ pu, 4×10⁷ pu, 1×10⁸ pu, 2×10⁸ pu, 3×10⁸ pu, 4×10⁸ pu, 1×10⁹ pu, 2×10⁹ pu, 3×10⁹ pu, 4×10⁹ pu, 1×10¹⁰ pu, 2×10¹⁰ pu, 3×10¹⁰ pu, 4×10¹⁰ pu, 1×10¹¹ pu, 2×10¹¹ pu, 3×10¹¹ pu, 4×10¹¹ pu, 1×10¹² pu, 2×10¹² pu, 3×10¹² pu, or 4×10¹² pu of the adenoviral vector.

Administration of the composition containing the adenovirus or adenoviral vector can be one component of a multistep regimen for inducing an immune response against Plasmodium falciparum in a mammal. In this respect, the method of inducing an immune response can further comprise administering to the mammal a boosting composition after administering the adenovirus or adenoviral vector to the mammal. In this embodiment, therefore, the immune response is “primed” upon administration of the composition containing the adenovirus or adenoviral vector, and is “boosted” upon administration of the boosting composition. Alternatively, the inventive method further comprises administering to the mammal a priming composition to the mammal prior to administering the adenovirus or adenoviral vector to the mammal. In this embodiment, therefore, the immune response is “primed” upon administration of the priming composition, and is “boosted” upon administration of the composition containing the adenovirus or adenoviral vector.

At least one of the priming composition and the boosting composition comprises an inventive adenovirus or adenoviral vector, while the other of the priming composition and the boosting composition can comprise the inventive adenovirus or adenoviral vector (which can be the same or different) or a different effective agent, though desirably a gene transfer vector that comprises a nucleic acid sequence encoding a Plasmodium antigen. Any gene transfer vector can be employed, including viral and non-viral gene transfer vectors. Examples of suitable viral gene transfer vectors include, but are not limited to, retroviral vectors, adeno-associated virus vectors, vaccinia virus vectors, herpesvirus vectors, parainfluenza-RSV chimeric vectors (PIV-RSV), and adenoviral vectors. Examples of suitable non-viral vectors include, but are not limited to, plasmids, liposomes, and molecular conjugates (e.g., transferrin). Preferably, the priming composition or the boosting composition comprises a plasmid or an adenoviral vector. Alternatively, an immune response can be primed or boosted by administration of a Plasmodium protein itself (e.g., an antigenic Plasmodium protein) with or without a suitable adjuvant (e.g., alum, QS-21, insulin-derived adjuvant, etc.), a live-attenuated Plasmodium parasite, and the like. When the priming composition or the boosting composition comprises an adenovirus or an adenoviral vector other than the inventive adenovirus or adenoviral vector, the adenovirus or adenoviral vector can be, or can be derived from, any adenovirus that infects a human or non-human animal, which are well known in the art. In this respect, the priming composition or the boosting composition can comprise a human adenoviral vector (e.g., serotype 5, 28, or 35), a simian adenoviral vector (such as described in, e.g., International Patent Application Publication WO 2011/057254), or a gorilla adenoviral vector. For example, a priming composition containing a human serotype 5 adenoviral vector can be administered to a human, followed by administration of a boosting composition containing the inventive adenovirus or adenoviral vector described herein (i.e., a “heterologous” prime-boost regimen). Alternatively, a priming composition containing the inventive adenovirus or adenoviral vector described herein can be administered to a human, followed by administration of a boosting composition containing a human serotype 5 adenoviral vector. In another embodiment, a priming composition containing the inventive adenovirus or adenoviral vector described herein can be administered to a human, followed by a second administration of the same composition (i.e., a “homologous” prime-boost regimen). One of ordinary skill in the art will appreciate that any combination of human and/or non-human adenoviruses and adenoviral vectors encoding one or more Plasmodium antigens can be employed as the priming and/or boosting compositions in conjunction with the inventive adenovirus or adenoviral vector described herein.

When a gene transfer vector other than the inventive adenovirus or adenoviral vector is utilized in the priming composition and/or the boosting composition, such gene transfer vector comprises at least one nucleic acid sequence encoding a Plasmodium antigen. The Plasmodium antigen encoded by the nucleic acid sequence of such a gene transfer vector can be the same as the Plasmodium antigen encoded by the inventive adenovirus or adenoviral vector. Alternatively, the Plasmodium antigen encoded by the nucleic acid sequence of such a gene transfer vector can be different from the Plasmodium antigen encoded by the inventive adenovirus or adenoviral vector. In one embodiment, such a gene transfer vector of the priming composition and/or the boosting composition comprises multiple (i.e., two or more) nucleic acid sequences encoding the same Plasmodium antigen, as described herein. In another embodiment, such a gene transfer vector of the priming composition and/or the boosting composition can comprise multiple nucleic acid sequences encoding two or more different Plasmodium antigens, as described herein.

Administration of the priming composition and the boosting composition can be separated by any suitable timeframe (e.g., at least any of about 1 week, 2 weeks, 4 weeks, 8 weeks, 12 weeks, 16 weeks, 24 weeks, 52 weeks, 2 years, and 5 years, or any range defined by any two of the foregoing values). The boosting composition preferably is administered to a mammal (e.g., a human) at least about 2 weeks (e.g., at least any of about 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 20 weeks, 24 weeks, 28 weeks, 35 weeks, 40 weeks, 50 weeks, 52 weeks, 2 years, and 5 years, or any range defined by any two of the foregoing values) following administration of the priming composition. More than one dose of priming composition and/or boosting composition can be provided in any suitable timeframe. The dose of the priming composition and boosting composition administered to the mammal depends on a number of factors, including the extent of any side-effects, the particular route of administration, and the like.

The following examples further illustrate the invention but, of course, should not be construed as in any way limiting its scope.

### Example 1

This example demonstrates the immunogenicity of an inventive adenoviral vector encoding a Plasmodium yoelii circumsporozoite protein (PyCSP) in mice.

Three adenoviruses were prepared. Specifically, adenoviruses having the nucleic acid sequence of SEQ ID NO: 22, SEQ ID NO: 52, or SEQ ID NO: 80 were modified by genetic engineering to (1) be rendered replication-deficient by deletion of the E1 region, and (2) express a codon-optimized version of the Plasmodium yoelii circumsporozoite protein which lacks the C-terminal gpi anchor domain (PyCSPo).

More specifically, a nucleic acid sequence encoding PyCSP was introduced between a CMVtetO promoter and the SV40 early polyA. The CMVtetO promoter combines the CMV immediate early high expression enhancer/promoter with tetracycline operator sites. Within this sequence is the viral enhancer, CAAT box, TATA box, two copies of the 20 nucleotide tetracycline operator sequence (tetO) from transposon Tn10, and the CMV transcription start site. The tetO sites are inactive in mammalian cells since tetracycline-based gene expression regulation is specific for a prokaryotic system (see, e.g., Blau et al., Proc. Natl. Acad. Sci USA., 96(3): 797-799 (1999)). The tetO sites inhibit transgene expression when the viral vector is propagated in a cell line in the presence of the tetracycline repressor. To further optimize the expression of the PyCSP nucleic acid sequence from the CMVtetO promoter, an artificial intron was created in the sequence by placing a splice donor and a splice acceptor sequence upstream of the PyCSP initiation codon. The resulting adenoviral vectors were designated GC44PyCSP.o, GC45PyCSP.o, and GC46PyCSP.o.

Balb/c mice were divided into groups (six mice per group), and each group was administered one of three doses of each of GC44PyCSP.o, GC45PyCSP.o, and GC46PyCSP.o (i.e., 1×10⁷ pu, 1×10⁸ pu, or 1×10⁹ pu), or an E1/E3/E4-deficient serotype 5 adenoviral vector comprising the PyCSP.o construct (“Ad5PyCSP.o”). Animals receiving an E1/E3/E4-deficient serotype 5 adenoviral vector lacking a transgene (“AdNull”) served as a negative control.

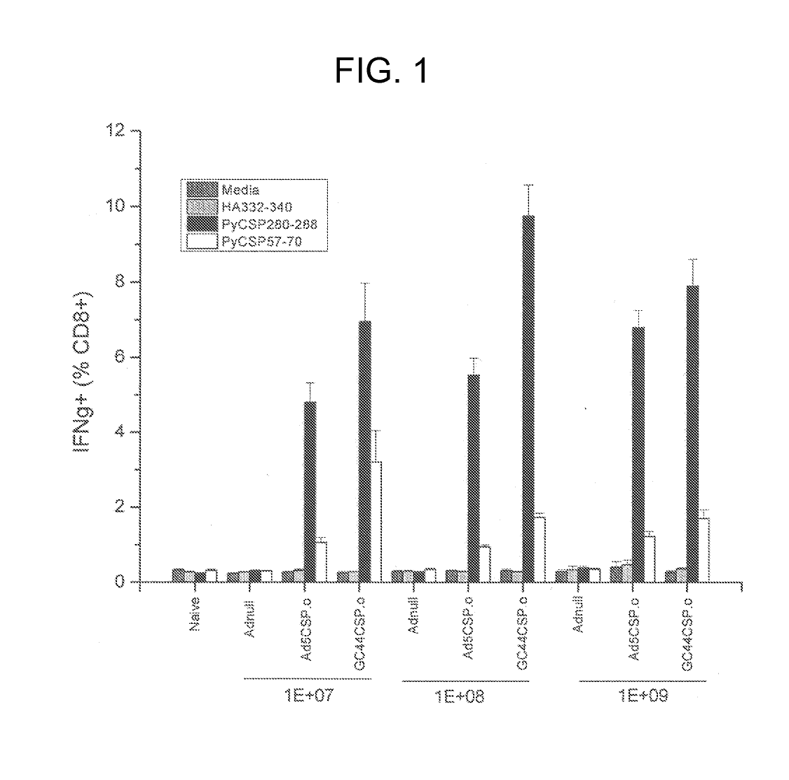

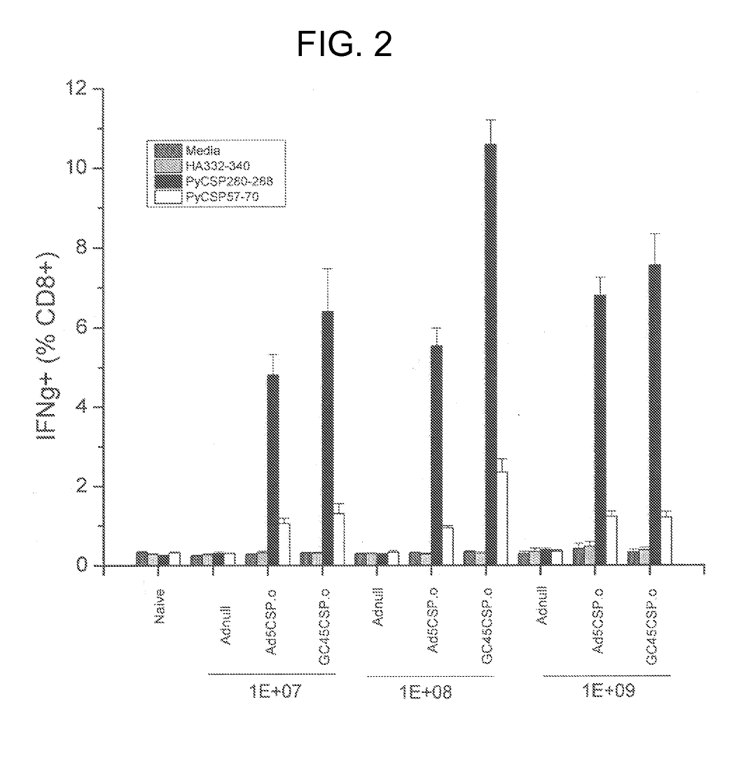

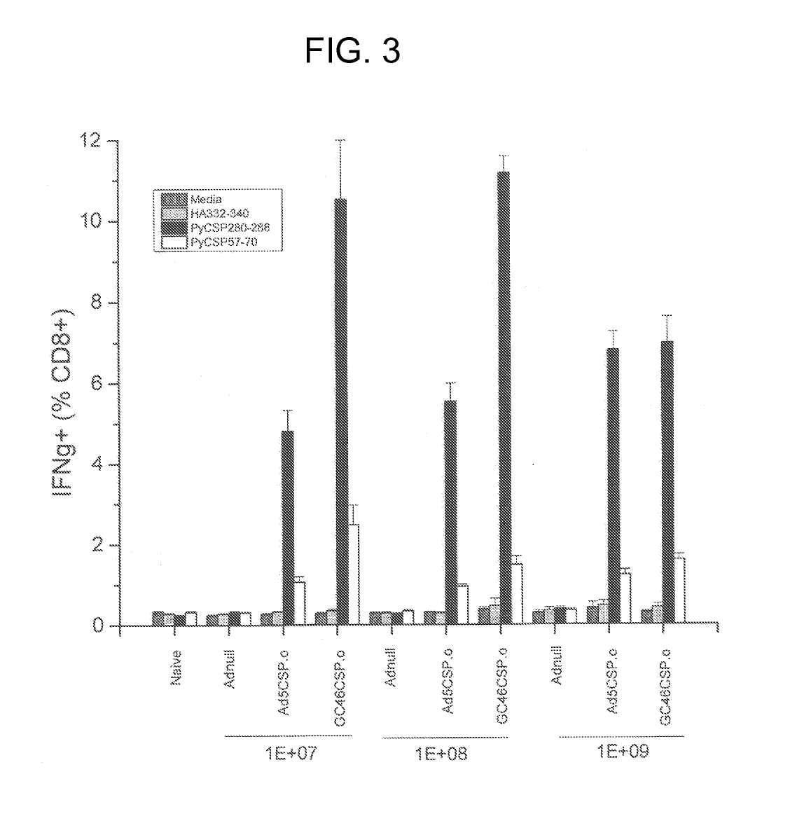

Three weeks following vector administration, T-cell responses were assessed by intracellular cytokine staining (ICS) of individual mice splenocytes. Targets were MHC-matched A20.2J cells pulsed with synthetic peptides representing the immunodominant CD8+ T cell epitope (PyCSP280-288), a subdominant epitope (PyCSP57-70), or a defined CD8+ T cell epitope for hemagglutinin (HA 332-340). The results of this analysis are set forth in FIGS. 1-3. At the 1×10⁸ pu dose level, GC44PyCSP.o and GC45PyCSP.o induced significantly higher CD8+ T cell levels in mice than did Ad5PyCSP.o. At the 1×10⁷ pu and 1×10⁸ pu dose levels, GC46PyCSP.o induced significantly higher CD8+ T cell levels in mice than did Ad5PyCSP.o. At the 1×10⁹ pu dose, GC46PyCSP.o and Ad5PyCSP.o induced similar CD8+ T cell responses in mice.

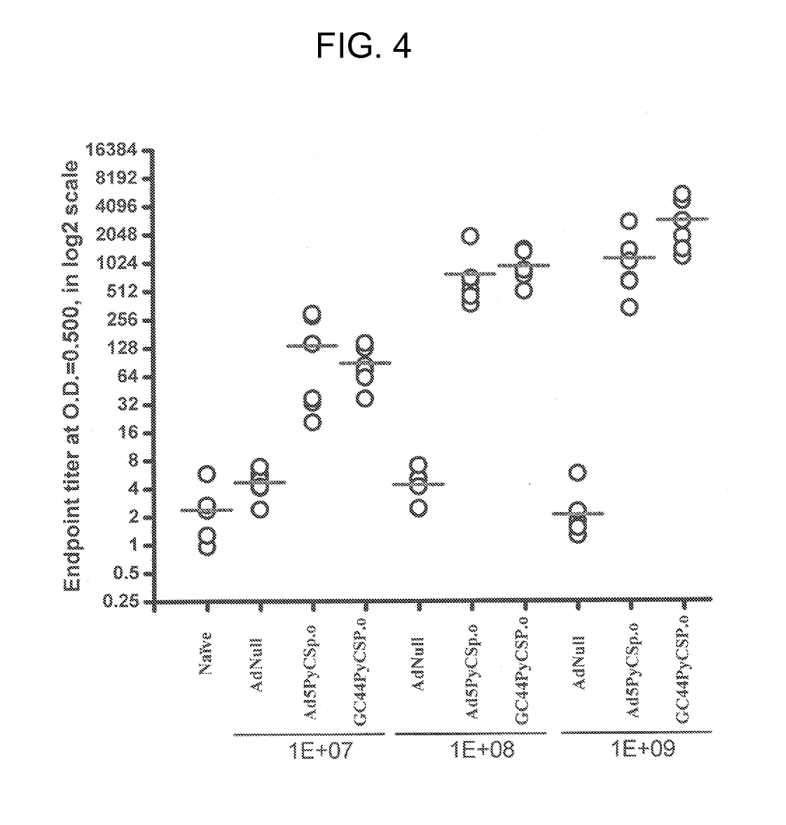

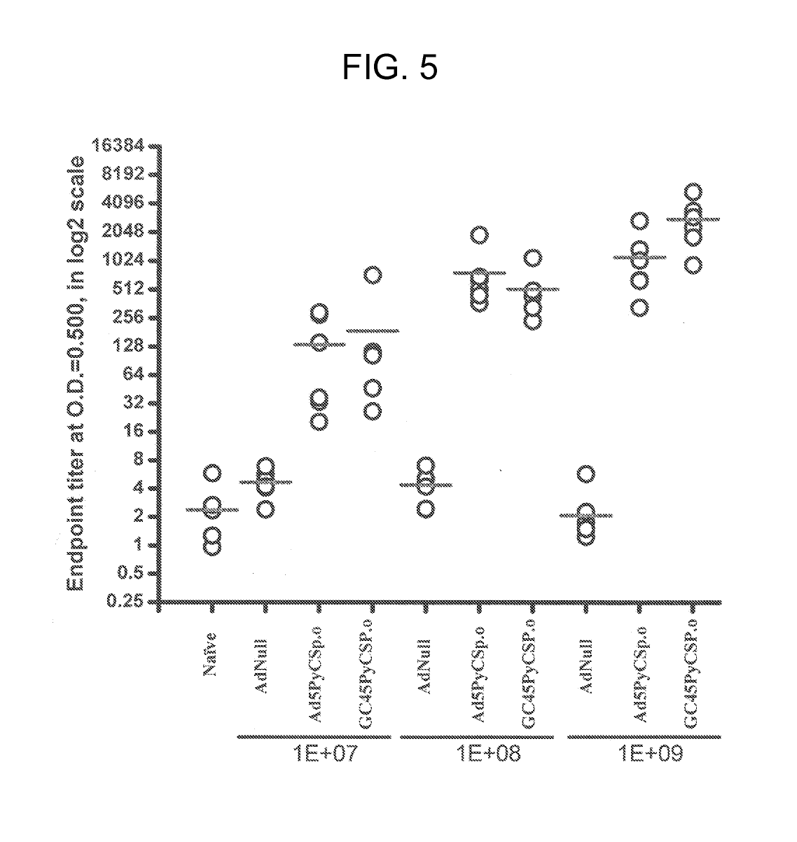

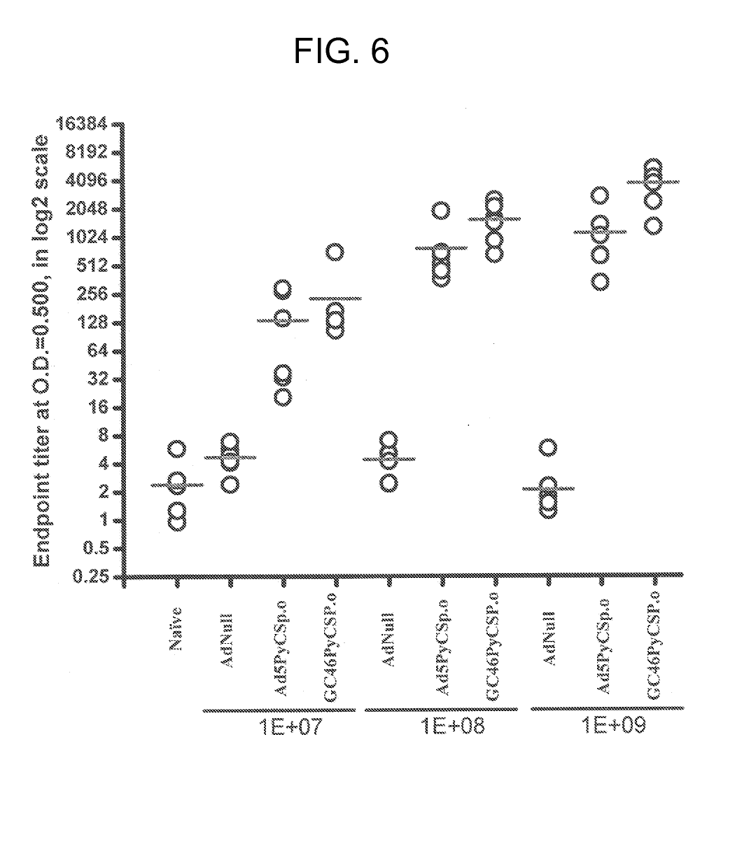

Antigen-specific antibody titers were evaluated in serum from the treated mice via ELISA. In particular, full length PyCSP protein was adsorbed to 96-well Immulon 4HBX plates at a concentration of 0.1 g/ml. Bound antibodies were detected using alkaline phosphatase labeled anti-mouse IgG (H+L), KPL diluted 1:1000. The antibody endpoint titer was calculated as the serum dilution that produced an OD₄₀₅ of 0.5 absorbance units in the ELISA assay using a four parameter curve fitting model in the Softmax software (Molecular Dynamics, Inc., Sunnyvale, Calif.). The result of this analysis are set forth in FIGS. 4-6. PyCSP-specific antibody titers induced by the GC44PyCSP.o, GC45PyCSP.o, and GC46PyCSP.o vectors at the 1×10⁹ pu dose level were significantly greater than the antibody titers induced by the Ad5PyCSP.o vector.

The results of this example demonstrate that the inventive adenoviral vector encoding a Plasmodium antigen is immunogenic in mice.

### Example 2

This example demonstrates that prime-boost immunization strategizes utilizing the inventive adenoviral vectors can protect against malaria infection in a mouse model.

Groups of Balb/c mice (n=14 per group) were injected IM with either 100 μg of a plasmid DNA expressing PyCSP (pExec.PyCSP) or with 1×10⁹ pu of one of the following adenoviral vectors: Ad5PyCSP.o, GC44PyCSP.o, GC45PyCSP.o, GC46PyCSP.o, or an E1/E3/E4-deficient serotype 28 adenoviral vector comprising the PyCSP.o construct (Ad28.PyCSP.o). Animals receiving an E1/E3/E4-deficient serotype 35 adenoviral vector lacking a transgene (“AdNull”) served as a negative control. 42 days following the priming injections, mice were boosted via intramuscular injection with 1×10⁹ pu of one of the aforementioned vectors according to the schedule set forth in Table 1.

TABLE 1

| Priming Vector | Dose | | Boosting Vector | Dose |
|---|---|---|---|---|
| Adnull | 1E+09 | pu | Adnull | 1E+09 pu |
| Ad5PyCSP.o | 1E+09 | pu | Ad5PyCSP.o | 1E+09 pu |
| pExec.PyCSP | 100 | μg | Ad5PyCSP.o | 1E+09 pu |
| Ad28.PyCSP.o | 1E+09 | pu | Ad5PyCSP.o | 1E+09 pu |
| GC44PyCSP.o | 1E+09 | pu | GC44PyCSP.o | 1E+09 pu |
| pExec.PyCSP | 100 | μg | GC44PyCSP.o | 1E+09 pu |
| Ad28.PyCSP.o | 1E+09 | pu | GC44PyCSP.o | 1E+09 pu |
| GC45PyCSP.o | 1E+09 | pu | GC45PyCSP.o | 1E+09 pu |
| pExec.PyCSP | 100 | μg | GC45PyCSP.o | 1E+09 pu |
| Ad28.PyCSP.o | 1E+09 | pu | GC45PyCSP.o | 1E+09 pu |
| GC46PyCSP.o | 1E+09 | pu | GC46PyCSP.o | 1E+09 pu |
| pExec.PyCSP | 100 | μg | GC46PyCSP.o | 1E+09 pu |
| Ad28.PyCSP.o | 1E+09 | pu | GC46PyCSP.o | 1E+09 pu |

Immunized mice were challenged with 100 live sporozoites/mouse two weeks after the boosting injection (Day 56) by tail vein injection (see, e.g., Limbach et al., Malaria Journal, 10: 65 (2011)). Tail vein bleeds were performed from day 61 to 70 (i.e., 5-14 days after live sporozoites challenge) to examine the clearance of parasite in the blood via parasitemia analysis. Due to evidence of a weak first challenge, mice were re-challenged nine weeks after the boost with 200 live sporozoites/mouse, and parasitemia analysis was performed from day 111 through day 119 (i.e., 5-14 days after second live sporozoites challenge) to confirm sterile protection. The results of the first and second challenge experiments are set forth in Table 2.

TABLE 2

| | First Challenge - 2 weeks post boost | | | | | Second Challenge - 9 weeks post boost | | | | | Single |
|---|---|---|---|---|---|---|---|---|---|---|---|
| | (# protected/#mice) | | | | | (# protected/#mice) | | | | | Challenge |
| Regimen | Ad5 | GC44 | GC45 | GC46 | Controls | Ad5 | GC44 | GC45 | GC46 | Controls | Controls |
| Ad Prime - | 14/14 | 13/14 | 14/14 | 14/14 | | 11/14 | 13/13 | 14/14 | 12/14 | | |
| Ad Boost | | | | | | | | | | | |
| (homologous) | | | | | | | | | | | |
| DNA Prime - | 14/14 | 14/14 | 13/14 | 14/14 | | 12/14 | 13/14 | 7/13 | 12/14 | | |
| Ad Boost | | | | | | | | | | | |
| Ad28 Prime - | 14/14 | 14/14 | 13/14 | 13/14 | | 12/14 | 13/14 | 9/13 | 11/13 | | |
| Ad Boost | | | | | | | | | | | |
| (heterologous) | | | | | | | | | | | |
| Naïve | | | | | 5/14 | | | | | 1/5 | 3/14 |
| Null | | | | | 5/14 | | | | | 0/5 | 3/14 |

The results of this example demonstrate that gorilla adenoviral vectors protected against malaria infection as well as human serotype 5 adenoviral vectors, and that homologous and heterologous prime-boost regimens provided comparable protection against malaria infection.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An adenovirus or adenoviral vector comprising (1) a nucleic acid sequence encoding one or more Plasmodium antigens and (2) one or more of the nucleic acid sequences selected from the group consisting of:
(a) the nucleic acid sequence of SEQ ID NO: 1,
(b) a nucleic acid sequence that is at least 98.5% identical to SEQ ID NO: 2,
(c) a nucleic acid sequence that is at least 90% identical to SEQ ID NO: 3,
(d) a nucleic acid sequence that is at least 80% identical to SEQ ID NO: 4, and
(e) a nucleic acid sequence that is at least 89% identical to SEQ ID NO: 5.

2. The adenovirus or adenoviral vector of claim 1, which comprises the nucleic acid sequence of SEQ ID NO: 1.

3. The adenovirus or adenoviral vector of claim 1, which comprises a nucleic acid sequence that is at least 98.5% identical to SEQ ID NO: 2.

4. The adenovirus or adenoviral vector of claim 3, which comprises the nucleic acid sequence of SEQ ID NO: 2.

5. The adenovirus or adenoviral vector of claim 1, which comprises a nucleic acid sequence that is at least 90% identical to SEQ ID NO: 3.

6. The adenovirus or adenoviral vector of claim 5, which comprises the nucleic acid sequence of SEQ ID NO: 3.

7. The adenovirus or adenoviral vector of claim 1, which comprises a nucleic acid sequence that is at least 80% identical to SEQ ID NO: 4.

8. The adenovirus or adenoviral vector of claim 7, which comprises the nucleic acid sequence of SEQ ID NO: 4.

9. The adenovirus or adenoviral vector of claim 1, which comprises a nucleic acid sequence that is at least 89% identical to SEQ ID NO: 5.

10. The adenovirus or adenoviral vector of claim 9, which comprises the nucleic acid sequence of SEQ ID NO: 5.

11. An adenovirus or adenoviral vector comprising (1) a nucleic acid sequence encoding one or more Plasmodium antigens and (2) one or more of the nucleic acid sequences selected from the group consisting of:
(a) a nucleic acid sequence that is at least 98.6% identical to SEQ ID NO: 6,
(b) a nucleic acid sequence that is at least 99.06% identical to SEQ ID NO: 7,
(c) a nucleic acid sequence that is at least 97.13% identical to SEQ ID NO: 8,
(d) a nucleic acid sequence that is at least 90.7% identical to SEQ ID NO: 9, and
(e) a nucleic acid sequence that is at least 96.6% identical to SEQ ID NO: 10.

12. The adenovirus or adenoviral vector of claim 11, which comprises a nucleic acid sequence that is at least 98.6% identical to SEQ ID NO: 6.

13. The adenovirus or adenoviral vector of claim 12, which comprises the nucleic acid sequence of SEQ ID NO: 6.

14. The adenovirus or adenoviral vector of claim 11, which comprises a nucleic acid sequence that is at least 99.06% identical to SEQ ID NO: 7.

15. The adenovirus or adenoviral vector of claim 14, which comprises the nucleic acid sequence of SEQ ID NO: 7.

16. The adenovirus or adenoviral vector of claim 11, which comprises a nucleic acid sequence that is at least 97.13% identical to SEQ ID NO: 8.

17. The adenovirus or adenoviral vector of claim 16, which comprises the nucleic acid sequence of SEQ ID NO: 8.

18. The adenovirus or adenoviral vector of claim 11, which comprises a nucleic acid sequence that is at least 90.7% identical to SEQ ID NO: 9.

19. The adenovirus or adenoviral vector of claim 18, which comprises the nucleic acid sequence of SEQ ID NO: 9.

20. The adenovirus or adenoviral vector of claim 1, which comprises a nucleic acid sequence that is at least 96.6% identical to SEQ ID NO: 10.

21. The adenovirus or adenoviral vector of claim 20, which comprises the nucleic acid sequence of SEQ ID NO: 10.

22. An adenovirus or adenoviral vector comprising (1) a nucleic acid sequence encoding one or more Plasmodium antigens and (2) one or more of the nucleic acid sequences selected from the group consisting of:
(a) a nucleic acid sequence comprising at least 121 contiguous nucleotides of SEQ ID NO: 6,
(b) a nucleic acid sequence comprising at least 462 contiguous nucleotides of SEQ ID NO: 7,
(c) a nucleic acid sequence comprising at least 234 contiguous nucleotides of SEQ ID NO: 8,
(d) a nucleic acid sequence comprising at least 606 contiguous nucleotides of SEQ ID NO: 9, and
(e) a nucleic acid sequence comprising at least 188 contiguous nucleotides of SEQ ID NO: 10.

23. The adenovirus or adenoviral vector of claim 22, which comprises a nucleic acid sequence comprising at least 121 contiguous nucleotides of SEQ ID NO: 6.

24. The adenovirus or adenoviral vector of claim 23, which comprises the nucleic acid sequence of SEQ ID NO: 6.

25. The adenovirus or adenoviral vector of claim 22, which comprises a nucleic acid sequence comprising at least 462 contiguous nucleotides of SEQ ID NO: 7

26. The adenovirus or adenoviral vector of claim 25, which comprises the nucleic acid sequence of SEQ ID NO: 7.

27. The adenovirus or adenoviral vector of claim 22, which comprises a nucleic acid sequence comprising at least 234 contiguous nucleotides of SEQ ID NO: 8.

28. The adenovirus or adenoviral vector of claim 27, which comprises the nucleic acid sequence of SEQ ID NO: 8.

29. The adenovirus or adenoviral vector of claim 22, which comprises a nucleic acid sequence comprising at least 606 contiguous nucleotides of SEQ ID NO: 9.

30. The adenovirus or adenoviral vector of claim 29, which comprises the nucleic acid sequence of SEQ ID NO: 9.

31. The adenovirus or adenoviral vector of claim 22, which comprises a nucleic acid sequence comprising at least 188 contiguous nucleotides of SEQ ID NO: 10.

32. The adenovirus or adenoviral vector of claim 31, which comprises the nucleic acid sequence of SEQ ID NO: 10.

33. An adenovirus or adenoviral vector comprising (1) a nucleic acid sequence encoding one or more Plasmodium antigens and (2) one or more of the amino acid sequences selected from the group consisting of:
(a) the amino acid sequence of SEQ ID NO: 11,
(b) an amino acid sequence that is at least 82% identical to SEQ ID NO: 13,
(c) an amino acid sequence that is at least 80% identical to SEQ ID NO: 14, and
(d) an amino acid sequence that is at least 83% identical to SEQ ID NO: 15.

34. The adenovirus or adenoviral vector of claim 33, which comprises the amino acid sequence of SEQ ID NO: 11.

35. The adenovirus or adenoviral vector of claim 33, which comprises an amino acid sequence that is at least 82% identical to SEQ ID NO: 13.

36. The adenovirus or adenoviral vector of claim 35, which comprises the amino acid sequence of SEQ ID NO: 13.

37. The adenovirus or adenoviral vector of claim 33, which comprises an amino acid sequence that is at least 80% identical to SEQ ID NO: 14.

38. The adenovirus or adenoviral vector of claim 37, which comprises the amino acid sequence of SEQ ID NO: 14.

39. The adenovirus or adenoviral vector of claim 33, which comprises an amino acid sequence that is at least 83% identical to SEQ ID NO: 15.

40. The adenovirus or adenoviral vector of claim 39, which comprises the amino acid sequence of SEQ ID NO: 15.

41. An adenovirus or adenoviral vector comprising (1) a nucleic acid sequence encoding one or more Plasmodium antigens and (2) one or more of the nucleic acid sequences selected from the group consisting of:
(a) a nucleic acid sequence encoding the amino acid sequence of SEQ ID NO: 11,
(b) a nucleic acid sequence encoding an amino acid sequence that is at least 99% identical to SEQ ID NO: 12,
(c) a nucleic acid sequence encoding an amino acid sequence that is at least 82% identical to SEQ ID NO: 13,
(d) a nucleic acid sequence encoding an amino acid sequence that is at least 80% identical to SEQ ID NO: 14, and
(e) a nucleic acid sequence encoding an amino acid sequence that is at least 83% identical to SEQ ID NO: 15.

42. The adenovirus or adenoviral vector of claim 41, which comprises a nucleic acid sequence encoding the amino acid sequence of SEQ ID NO: 11.

43. The adenovirus or adenoviral vector of claim 41, which comprises a nucleic acid sequence encoding an amino acid sequence that is at least 99% identical to SEQ ID NO: 12.

44. The adenovirus or adenoviral vector of claim 43, which comprises a nucleic acid sequence encoding the amino acid sequence of SEQ ID NO: 12.

45. The adenovirus or adenoviral vector of claim 41, which comprises a nucleic acid sequence encoding an amino acid sequence that is at least 82% identical to SEQ ID NO: 13.

46. The adenovirus or adenoviral vector of claim 45, which comprises a nucleic acid sequence encoding the amino acid sequence of SEQ ID NO: 13.

47. The adenovirus or adenoviral vector of claim 41, which comprises a nucleic acid sequence encoding an amino acid sequence that is at least 80% identical to SEQ ID NO: 14.

48. The adenovirus or adenoviral vector of claim 47, which comprises a nucleic acid sequence encoding the amino acid sequence of SEQ ID NO: 14.

49. The adenovirus or adenoviral vector of claim 41, which comprises a nucleic acid sequence encoding an amino acid sequence that is at least 83% identical to SEQ ID NO: 15.

50. The adenovirus or adenoviral vector of claim 49, which comprises a nucleic acid sequence encoding the amino acid sequence of SEQ ID NO: 15.

51. An adenovirus or adenoviral vector comprising (1) a nucleic acid sequence encoding one or more Plasmodium antigens and (2) one or more of the amino acid sequences selected from the group consisting of:
(a) the amino acid sequence of SEQ ID NO: 16,
(b) an amino acid sequence that is at least 97.8% identical to SEQ ID NO: 18,
(c) an amino acid that is at least 93.4% identical to SEQ ID NO: 19, and
(d) an amino acid sequence that is at least 98.2% identical to SEQ ID NO: 20.

52. The adenovirus or adenoviral vector of claim 51, which comprises the amino acid sequence of SEQ ID NO: 16.

53. The adenovirus or adenoviral vector of claim 51, which comprises an amino acid sequence that is at least 97.8% identical to SEQ ID NO: 18.

54. The adenovirus or adenoviral vector of claim 53, which comprises the amino acid sequence of SEQ ID NO: 18.

55. The adenovirus or adenoviral vector of claim 51, which comprises an amino acid sequence that is at least 93.4% identical to SEQ ID NO: 19.

56. The adenovirus or adenoviral vector of claim 55, which comprises the amino acid sequence of SEQ ID NO: 19.

57. The adenovirus or adenoviral vector of claim 51, which comprises an amino acid sequence that is at least 98.2% identical to SEQ ID NO: 20.

58. The adenovirus or adenoviral vector of claim 57, which comprises the amino acid sequence of SEQ ID NO: 20.

59. An adenovirus or adenoviral vector comprising (1) a nucleic acid sequence encoding one or more Plasmodium antigens and (2) one or more of the nucleic acid sequences selected from the group consisting of:
(a) a nucleic acid sequence encoding the amino acid sequence of SEQ ID NO: 16,
(b) a nucleic acid sequence encoding an amino acid sequence that is at least 99.78% identical to SEQ ID NO: 17,
(c) a nucleic acid sequence encoding an amino acid sequence that is at least 97.8% identical to SEQ ID NO: 18,
(d) a nucleic acid sequence encoding an amino acid that is at least 93.4% identical to SEQ ID NO: 19, and
(e) a nucleic acid sequence encoding an amino acid sequence that is at least 98.2% identical to SEQ ID NO: 20.

60. The adenovirus or adenoviral vector of claim 59, which comprises a nucleic acid sequence encoding the amino acid sequence of SEQ ID NO: 16.

61. The adenovirus or adenoviral vector of claim 59, which comprises a nucleic acid sequence encoding an amino acid sequence that is at least 99.78% identical to SEQ ID NO: 17.

62. The adenovirus or adenoviral vector of claim 61, which comprises a nucleic acid sequence encoding the amino acid sequence of SEQ ID NO: 17.

63. The adenovirus or adenoviral vector of claim 59, which comprises a nucleic acid sequence encoding an amino acid sequence that is at least 97.8% identical to SEQ ID NO: 18.

64. The adenovirus or adenoviral vector of claim 63, which comprises a nucleic acid sequence encoding the amino acid sequence of SEQ ID NO: 18.

65. The adenovirus or adenoviral vector of claim 59, which comprises a nucleic acid sequence encoding an amino acid sequence that is at least 93.4% identical to SEQ ID NO: 19.

66. The adenovirus or adenoviral vector of claim 65, which comprises a nucleic acid sequence encoding the amino acid sequence of SEQ ID NO: 19.

67. The adenovirus or adenoviral vector of claim 59, which comprises an amino acid sequence that is at least 98.2% identical to SEQ ID NO: 20.

68. The adenovirus or adenoviral vector of claim 67, which comprises the amino acid sequence of SEQ ID NO: 20.

69. An adenovirus or adenoviral vector comprising (1) a nucleic acid sequence encoding one or more Plasmodium antigens and (2) one or more of the amino acid sequences selected from the group consisting of:
(a) an amino acid sequence comprising at least 89 contiguous amino acid residues of SEQ ID NO: 16,
(b) an amino acid sequence comprising at least 247 contiguous amino acid residues of SEQ ID NO: 18,
(c) an amino acid sequence comprising at least 370 contiguous amino acid residues of SEQ ID NO: 19, and
(d) an amino acid sequence comprising at least 192 contiguous amino acid residues of SEQ ID NO: 20.

70. The adenovirus or adenoviral vector of claim 69, which comprises an amino acid sequence comprising at least 89 contiguous amino acid residues of SEQ ID NO: 16.

71. The adenovirus or adenoviral vector of claim 70, which comprises the amino acid sequence of SEQ ID NO: 16.

72. The adenovirus or adenoviral vector of claim 69, which comprises an amino acid sequence comprising at least 247 contiguous amino acid residues of SEQ ID NO: 18.

73. The adenovirus or adenoviral vector of claim 72, which comprises the amino acid sequence of SEQ ID NO: 18.

74. The adenovirus or adenoviral vector of claim 69, which comprises an amino acid sequence comprising at least 370 contiguous amino acid residues of SEQ ID NO: 19.

75. The adenovirus or adenoviral vector of claim 74, which comprises the amino acid sequence of SEQ ID NO: 19.

76. The adenovirus or adenoviral vector of claim 69, which comprises an amino acid sequence comprising at least 192 contiguous amino acid residues of SEQ ID NO: 20.

77. The adenovirus or adenoviral vector of claim 76, which comprises the amino acid sequence of SEQ ID NO: 20.

78. An adenovirus or adenoviral vector comprising (1) a nucleic acid sequence encoding one or more Plasmodium antigens and (2) one or more of the nucleic acid sequences selected from the group consisting of:
(a) a nucleic acid sequence encoding an amino acid sequence comprising at least 89 contiguous amino acid residues of SEQ ID NO: 16,
(b) a nucleic acid sequence encoding an amino acid sequence comprising at least 428 contiguous amino acid residues of SEQ ID NO: 17,
(c) a nucleic acid sequence encoding an amino acid sequence comprising at least 247 contiguous amino acid residues of SEQ ID NO: 18,
(d) a nucleic acid sequence encoding an amino acid sequence comprising at least 370 contiguous amino acid residues of SEQ ID NO: 19, and
(e) a nucleic acid sequence encoding an amino acid sequence comprising at least 192 contiguous amino acid residues of SEQ ID NO: 20.

79. The adenovirus or adenoviral vector of claim 78, which comprises a nucleic acid sequence encoding an amino acid sequence comprising at least 89 contiguous amino acid residues of SEQ ID NO: 16.

80. The adenovirus or adenoviral vector of claim 79, which comprises a nucleic acid sequence encoding the amino acid sequence of SEQ ID NO: 16.

81. The adenovirus or adenoviral vector of claim 78, which comprises a nucleic acid sequence encoding an amino acid sequence comprising at least 428 contiguous amino acid residues of SEQ ID NO: 17.

82. The adenovirus or adenoviral vector of claim 81, which comprises a nucleic acid sequence encoding the amino acid sequence of SEQ ID NO: 17.

83. The adenovirus or adenoviral vector of claim 78, which comprises a nucleic acid sequence encoding an amino acid sequence comprising at least 247 contiguous amino acid residues of SEQ ID NO: 18.

84. The adenovirus or adenoviral vector of claim 83, which comprises a nucleic acid sequence encoding the amino acid sequence of SEQ ID NO: 18.

85. The adenovirus or adenoviral vector of claim 78, which comprises a nucleic acid sequence encoding an amino acid sequence comprising at least 370 contiguous amino acid residues of SEQ ID NO: 19.

86. The adenovirus or adenoviral vector of claim 85, which comprises a nucleic acid sequence encoding the amino acid sequence of SEQ ID NO: 19.

87. The adenovirus or adenoviral vector of claim 78, which comprises a nucleic acid sequence encoding an amino acid sequence comprising at least 192 contiguous amino acid residues of SEQ ID NO: 20.

88. The adenovirus or adenoviral vector of claim 87, which comprises a nucleic acid sequence encoding the amino acid sequence of SEQ ID NO: 20.

89. An adenovirus or adenoviral vector comprising (1) a nucleic acid sequence encoding one or more Plasmodium antigens and (2) one or more of the nucleic acid sequences selected from the group consisting of:
(a) a nucleic acid sequence that is at least 96% identical to SEQ ID NO: 25,
(b) a nucleic acid sequence that is at least 99% identical to SEQ ID NO: 26,
(c) a nucleic acid sequence that is at least 80% identical to SEQ ID NO: 27,
(d) a nucleic acid sequence that is at least 80% identical to SEQ ID NO: 28, and
(e) a nucleic acid sequence that is at least 85.4% identical to SEQ ID NO: 29.

90. The adenovirus or adenoviral vector of claim 89, which comprises a nucleic acid sequence that is at least 96% identical to SEQ ID NO: 25.

91. The adenovirus or adenoviral vector of claim 90, which comprises the nucleic acid sequence of SEQ ID NO: 25.

92. The adenovirus or adenoviral vector of claim 89, which comprises a nucleic acid sequence that is at least 99% identical to SEQ ID NO: 26.

93. The adenovirus or adenoviral vector of claim 89, which comprises a nucleic acid sequence that is at least 85.4% identical to SEQ ID NO: 29.

99. The adenovirus or adenoviral vector of claim 98, which comprises the nucleic acid sequence of SEQ ID NO: 29.

100. An adenovirus or adenoviral vector comprising (1) a nucleic acid sequence encoding one or more Plasmodium antigens and (2) one or more of the nucleic acid sequences selected from the group consisting of:
(a) a nucleic acid sequence that is at least 98.7% identical to SEQ ID NO: 30,
(b) a nucleic acid sequence that is at least 98.9% identical to SEQ ID NO: 31,
(c) a nucleic acid sequence that is at least 99.4% identical to SEQ ID NO: 32,
(d) a nucleic acid sequence that is at least 99.1% identical to SEQ ID NO: 33,
(e) a nucleic acid sequence that is at least 81.25% identical to SEQ ID NO: 34,
(f) a nucleic acid sequence that is at least 90.83% identical to SEQ ID NO: 35, and
(g) a nucleic acid sequence that is at least 82.5% identical to SEQ ID NO: 36.

101. The adenovirus or adenoviral vector of claim 100, which comprises a nucleic acid sequence that is at least 98.7% identical to SEQ ID NO: 30.

102. The adenovirus or adenoviral vector of claim 101, which comprises the nucleic acid sequence of SEQ ID NO: 30.

103. The adenovirus or adenoviral vector of claim 100, which comprises a nucleic acid sequence that is at least 98.9% identical to SEQ ID NO: 31.

104. The adenovirus or adenoviral vector of claim 103, which comprises the nucleic acid sequence of SEQ ID NO: 31.

105. The adenovirus or adenoviral vector of claim 100, which comprises a nucleic acid sequence that is at least 99.4% identical to SEQ ID NO: 32.

106. The adenovirus or adenoviral vector of claim 105, which comprises the nucleic acid sequence of SEQ ID NO: 32.

107. The adenovirus or adenoviral vector of claim 100, which comprises a nucleic acid sequence that is at least 99.1% identical to SEQ ID NO: 33.

108. The adenovirus or adenoviral vector of claim 107, which comprises the nucleic acid sequence of SEQ ID NO: 33.

109. The adenovirus or adenoviral vector of claim 100, which comprises a nucleic acid sequence that is at least 81.25% identical to SEQ ID NO: 34.

110. The adenovirus or adenoviral vector of claim 109, which comprises the nucleic acid sequence of SEQ ID NO: 34.

111. The adenovirus or adenoviral vector of claim 100, which comprises a nucleic acid sequence that is at least 90.83% identical to SEQ ID NO: 35.

112. The adenovirus or adenoviral vector of claim 111, which comprises the nucleic acid sequence of SEQ ID NO: 35.

113. The adenovirus or adenoviral vector of claim 100, which comprises a nucleic acid sequence that is at least 82.5% identical to SEQ ID NO: 36.

114. The adenovirus or adenoviral vector of claim 113, which comprises the nucleic acid sequence of SEQ ID NO: 36.

115. An adenovirus or adenoviral vector comprising (1) a nucleic acid sequence encoding one or more Plasmodium antigens and (2) one or more of the nucleic acid sequences selected from the group consisting of:
(a) a nucleic acid sequence comprising at least 162 contiguous nucleotides of SEQ ID NO: 30,
(b) a nucleic acid sequence comprising at least 162 contiguous nucleotides of SEQ ID NO: 31,
(c) a nucleic acid sequence comprising at least 121 contiguous nucleotides of SEQ ID NO: 32,
(d) a nucleic acid sequence comprising at least 531 contiguous nucleotides of SEQ ID NO: 33,
(e) a nucleic acid sequence comprising at least 156 contiguous nucleotides of SEQ ID NO: 34,
(f) a nucleic acid sequence comprising at least 192 contiguous nucleotides of SEQ ID NO: 35, and
(g) a nucleic acid sequence comprising at least 84 contiguous nucleotides of SEQ ID NO: 36.

116. The adenovirus or adenoviral vector of claim 115, which comprises a nucleic acid sequence comprising at least 162 contiguous nucleotides of SEQ ID NO: 30.

117. The adenovirus or adenoviral vector of claim 116, which comprises the nucleic acid sequence of SEQ ID NO: 30.

118. The adenovirus or adenoviral vector of claim 115, which comprises a nucleic acid sequence comprising at least 162 contiguous nucleotides of SEQ ID NO: 31.

119. The adenovirus or adenoviral vector of claim 118, which comprises the nucleic acid sequence of SEQ ID NO: 31.

120. The adenovirus or adenoviral vector of claim 115, which comprises a nucleic acid sequence comprising at least 121 contiguous nucleotides of SEQ ID NO: 32.

121. The adenovirus or adenoviral vector of claim 120, which comprises the nucleic acid sequence of SEQ ID NO: 32.

122. The adenovirus or adenoviral vector of claim 115, which comprises a nucleic acid sequence comprising at least 531 contiguous nucleotides of SEQ ID NO: 33.

123. The adenovirus or adenoviral vector of claim 122, which comprises the nucleic acid sequence of SEQ ID NO: 33.

124. The adenovirus or adenoviral vector of claim 115, which comprises a nucleic acid sequence comprising at least 156 contiguous nucleotides of SEQ ID NO: 34.

125. The adenovirus or adenoviral vector of claim 124, which comprises the nucleic acid sequence of SEQ ID NO: 34.

126. The adenovirus or adenoviral vector of claim 115, which comprises a nucleic acid sequence comprising at least 192 contiguous nucleotides of SEQ ID NO: 35.

127. The adenovirus or adenoviral vector of claim 126, which comprises the nucleic acid sequence of SEQ ID NO: 35.

128. The adenovirus or adenoviral vector of claim 115, which comprises a nucleic acid sequence comprising at least 84 contiguous nucleotides of SEQ ID NO: 36.

129. The adenovirus or adenoviral vector of claim 128, which comprises the nucleic acid sequence of SEQ ID NO: 36.

130. An adenovirus or adenoviral vector comprising (1) a nucleic acid sequence encoding one or more Plasmodium antigens and (2) one or more of the amino acid sequences selected from the group consisting of:
(a) an amino acid sequence that is at least 85% identical to SEQ ID NO: 38,
(b) an amino acid sequence that is at least 80% identical to SEQ ID NO: 39, and
(c) an amino acid sequence that is at least 80% identical to SEQ ID NO: 40.

131. The adenovirus or adenoviral vector of claim 130, which comprises an amino acid sequence that is at least 85% identical to SEQ ID NO: 38.

132. The adenovirus or adenoviral vector of claim 131, which comprises the amino acid sequence of SEQ ID NO: 38.

133. The adenovirus or adenoviral vector of claim 130, which comprises an amino acid sequence that is at least 80% identical to SEQ ID NO: 39.

134. The adenovirus or adenoviral vector of claim 133, which comprises the amino acid sequence of SEQ ID NO: 39.

135. The adenovirus or adenoviral vector of claim 130, which comprises an amino acid sequence that is at least 80% identical to SEQ ID NO: 40.

136. The adenovirus or adenoviral vector of claim 135, which comprises the amino acid sequence of SEQ ID NO: 40.

137. An adenovirus or adenoviral vector comprising (1) a nucleic acid sequence encoding one or more Plasmodium antigens and (2) one or more of the nucleic acid sequences selected from the group consisting of:
(a) a nucleic acid sequence encoding an amino acid sequence that is at least 99.7% identical to SEQ ID NO: 37,
(b) a nucleic acid sequence encoding an amino acid sequence that is at least 85% identical to SEQ ID NO: 38,
(c) a nucleic acid sequence encoding an amino acid sequence that is at least 80% identical to SEQ ID NO: 39, and
(d) a nucleic acid sequence encoding an amino acid sequence that is at least 80% identical to SEQ ID NO: 40.

138. The adenovirus or adenoviral vector of claim 137, which comprises a nucleic acid sequence encoding an amino acid sequence that is at least 99.7% identical to SEQ ID NO: 37.

139. The adenovirus or adenoviral vector of claim 138, which comprises a nucleic acid sequence encoding the amino acid sequence of SEQ ID NO: 37.

140. The adenovirus or adenoviral vector of claim 137, which comprises a nucleic acid sequence encoding an amino acid sequence that is at least 85% identical to SEQ ID NO: 38.

141. The adenovirus or adenoviral vector of claim 140, which comprises a nucleic acid sequence encoding the amino acid sequence of SEQ ID NO: 38.

142. The adenovirus or adenoviral vector of claim 137, which comprises a nucleic acid sequence encoding an amino acid sequence that is at least 80% identical to SEQ ID NO: 39.

143. The adenovirus or adenoviral vector of claim 142, which comprises a nucleic acid sequence encoding the amino acid sequence of SEQ ID NO: 39.

144. The adenovirus or adenoviral vector of claim 137, which comprises a nucleic acid sequence encoding an amino acid sequence that is at least 80% identical to SEQ ID NO: 40.

145. The adenovirus or adenoviral vector of claim 144, which comprises a nucleic acid sequence encoding the amino acid sequence of SEQ ID NO: 40.

146. An adenovirus or adenoviral vector comprising (1) a nucleic acid sequence encoding one or more Plasmodium antigens and (2) one or more of the amino acid sequences selected from the group consisting of:
(a) an amino acid sequence that is at least 99% identical to SEQ ID NO: 42,
(b) an amino acid sequence that is at least 81.4% identical to SEQ ID NO: 44,
(c) an amino acid sequence that is at least 91.3% identical to SEQ ID NO: 45, and
(d) an amino acid sequence that is at least 83.4% identical to SEQ ID NO: 46.

147. The adenovirus or adenoviral vector of claim 146, which comprises an amino acid sequence that is at least 99% identical to SEQ ID NO: 42.

148. The adenovirus or adenoviral vector of claim 147, which comprises the amino acid sequence of SEQ ID NO: 42.

149. The adenovirus or adenoviral vector of claim 146, which comprises an amino acid sequence that is at least 81.4% identical to SEQ ID NO: 44.

150. The adenovirus or adenoviral vector of claim 149, which comprises the amino acid sequence of SEQ ID NO: 44.

151. The adenovirus or adenoviral vector of claim 146, which comprises an amino acid sequence that is at least 91.3% identical to SEQ ID NO: 45.

152. The adenovirus or adenoviral vector of claim 151, which comprises the amino acid sequence of SEQ ID NO: 45.

153. The adenovirus or adenoviral vector of claim 146, which comprises an amino acid sequence that is at least 83.4% identical to SEQ ID NO: 46.

154. The adenovirus or adenoviral vector of claim 153, which comprises the amino acid sequence of SEQ ID NO: 46.

155. An adenovirus or adenoviral vector comprising (1) a nucleic acid sequence encoding one or more Plasmodium antigens and (2) one or more of the nucleic acid sequences selected from the group consisting of:
(a) a nucleic acid sequence encoding an amino acid sequence that is at least 99% identical to SEQ ID NO: 42,
(b) a nucleic acid sequence encoding an amino acid sequence that is at least 99.75% identical to SEQ ID NO: 43,
(c) a nucleic acid sequence encoding an amino acid sequence that is at least 81.4% identical to SEQ ID NO: 44,
(d) a nucleic acid sequence encoding an amino acid sequence that is at least 91.3% identical to SEQ ID NO: 45,
(e) a nucleic acid sequence encoding an amino acid sequence that is at least 83.4% identical to SEQ ID NO: 46.

156. The adenovirus or adenoviral vector of claim 155, which comprises a nucleic acid sequence encoding an amino acid sequence that is at least 99% identical to SEQ ID NO: 42.

157. The adenovirus or adenoviral vector of claim 156, which comprises a nucleic acid sequence encoding the amino acid sequence of SEQ ID NO: 42.

158. The adenovirus or adenoviral vector of claim 155, which comprises a nucleic acid sequence encoding an amino acid sequence that is at least 99.75% identical to SEQ ID NO: 43.

159. The adenovirus or adenoviral vector of claim 158, which comprises a nucleic acid sequence encoding the amino acid sequence of SEQ ID NO: 43.

160. The adenovirus or adenoviral vector of claim 155, which comprises a nucleic acid sequence encoding an amino acid sequence that is at least 81.4% identical to SEQ ID NO: 44.

161. The adenovirus or adenoviral vector of claim 160, which comprises a nucleic acid sequence encoding the amino acid sequence of SEQ ID NO: 44.

162. The adenovirus or adenoviral vector of claim 155, which comprises a nucleic acid sequence encoding an amino acid sequence that is at least 91.3% identical to SEQ ID NO: 45.

163. The adenovirus or adenoviral vector of claim 162, which comprises a nucleic acid sequence encoding the amino acid sequence of SEQ ID NO: 45.

164. The adenovirus or adenoviral vector of claim 155, which comprises a nucleic acid sequence encoding an amino acid sequence that is at least 83.4% identical to SEQ ID NO: 46.

165. The adenovirus or adenoviral vector of claim 164, which comprises a nucleic acid sequence encoding the amino acid sequence of SEQ ID NO: 46.

166. An adenovirus or adenoviral vector comprising (1) a nucleic acid sequence encoding one or more Plasmodium antigens and (2) one or more of the amino acid sequences selected from the group consisting of:
(a) an amino acid sequence comprising at least 89 contiguous amino acid residues of SEQ ID NO: 42,
(b) an amino acid sequence comprising at least 114 contiguous amino acid residues of SEQ ID NO: 44,
(c) an amino acid sequence comprising at least 231 contiguous amino acid residues of SEQ ID NO: 45, and
(d) an amino acid sequence comprising at least 30 contiguous amino acid residues of SEQ ID NO: 46.

167. The adenovirus or adenoviral vector of claim 166, which comprises an amino acid sequence comprising at least 89 contiguous amino acid residues of SEQ ID NO: 42.

168. The adenovirus or adenoviral vector of claim 167, which comprises the amino acid sequence of SEQ ID NO: 42.

169. The adenovirus or adenoviral vector of claim 166, which comprises an amino acid sequence comprising at least 114 contiguous amino acid residues of SEQ ID NO: 44.

170. The adenovirus or adenoviral vector of claim 169, which comprises the amino acid sequence of SEQ ID NO: 44.

171. The adenovirus or adenoviral vector of claim 166, which comprises an amino acid sequence comprising at least 231 contiguous amino acid residues of SEQ ID NO: 45.

172. The adenovirus or adenoviral vector of claim 171, which comprises the amino acid sequence of SEQ ID NO: 45.

173. The adenovirus or adenoviral vector of claim 166, which comprises an amino acid sequence comprising at least 30 contiguous amino acid residues of SEQ ID NO: 46.

174. The adenovirus or adenoviral vector of claim 173, which comprises the amino acid sequence of SEQ ID NO: 46.

175. An adenovirus or adenoviral vector comprising (1) a nucleic acid sequence encoding one or more Plasmodium antigens and (2) one or more of the nucleic acid sequences selected from the group consisting of:
(a) a nucleic acid sequence encoding an amino acid sequence comprising at least 89 contiguous amino acid residues of SEQ ID NO: 42,
(b) a nucleic acid sequence encoding an amino acid sequence comprising at least 428 contiguous amino acid residues of SEQ ID NO: 43,
(c) a nucleic acid sequence encoding an amino acid sequence comprising at least 114 contiguous amino acid residues of SEQ ID NO: 44,
(d) a nucleic acid sequence encoding an amino acid sequence comprising at least 231 contiguous amino acid residues of SEQ ID NO: 45, and
(e) a nucleic acid sequence encoding an amino acid sequence comprising at least 30 contiguous amino acid residues of SEQ ID NO: 46.

176. The adenovirus or adenoviral vector of claim 175, which comprises a nucleic acid sequence encoding an amino acid sequence comprising at least 89 contiguous amino acid residues of SEQ ID NO: 42.

177. The adenovirus or adenoviral vector of claim 176, which comprises a nucleic acid sequence encoding the amino acid sequence of SEQ ID NO: 42.

178. The adenovirus or adenoviral vector of claim 175, which comprises a nucleic acid sequence encoding an amino acid sequence comprising at least 428 contiguous amino acid residues of SEQ ID NO: 43

179. The adenovirus or adenoviral vector of claim 178, which comprises a nucleic acid sequence encoding the amino acid sequence of SEQ ID NO: 43.

180. The adenovirus or adenoviral vector of claim 175, which comprises a nucleic acid sequence encoding an amino acid sequence comprising at least 114 contiguous amino acid residues of SEQ ID NO: 44.

181. The adenovirus or adenoviral vector of claim 180, which comprises a nucleic acid sequence encoding the amino acid sequence of SEQ ID NO: 44.

182. The adenovirus or adenoviral vector of claim 175, which comprises a nucleic acid sequence encoding an amino acid sequence comprising at least 231 contiguous amino acid residues of SEQ ID NO: 45.

183. The adenovirus or adenoviral vector of claim 182, which comprises a nucleic acid sequence encoding the amino acid sequence of SEQ ID NO: 45.

184. The adenovirus or adenoviral vector of claim 175, which comprises a nucleic acid sequence encoding an amino acid sequence comprising at least 30 contiguous amino acid residues of SEQ ID NO: 46.

185. The adenovirus or adenoviral vector of claim 184, which comprises a nucleic acid sequence encoding the amino acid sequence of SEQ ID NO: 46.

186. An adenovirus or adenoviral vector comprising (1) a nucleic acid sequence encoding one or more Plasmodium antigens and (2) one or more of the nucleic acid sequences selected from the group consisting of:
(a) a nucleic acid sequence that is at least 97% identical to SEQ ID NO: 59,
(b) a nucleic acid sequence that is at least 97.5% identical to SEQ ID NO: 60,
(c) a nucleic acid sequence that is at least 80% identical to SEQ ID NO: 61,
(d) a nucleic acid sequence that is at least 96% identical to SEQ ID NO: 62, and
(e) a nucleic acid sequence that is at least 96% identical to SEQ ID NO: 63.

187. The adenovirus or adenoviral vector of claim 186, which comprises a nucleic acid sequence that is at least 97% identical to SEQ ID NO: 59.

188. The adenovirus or adenoviral vector of claim 187, which comprises the nucleic acid sequence of SEQ ID NO: 59.

189. The adenovirus or adenoviral vector of claim 186, which comprises a nucleic acid sequence that is at least 97.5% identical to SEQ ID NO: 60.

190. The adenovirus or adenoviral vector of claim 189, which comprises the nucleic acid sequence of SEQ ID NO: 60.

191. The adenovirus or adenoviral vector of claim 186, which comprises a nucleic acid sequence that is at least 80% identical to SEQ ID NO: 61.

192. The adenovirus or adenoviral vector of claim 191, which comprises the nucleic acid sequence of SEQ ID NO: 61.

193. The adenovirus or adenoviral vector of claim 186, which comprises a nucleic acid sequence that is at least 96% identical to SEQ ID NO: 62.

194. The adenovirus or adenoviral vector of claim 193, which comprises the nucleic acid sequence of SEQ ID NO: 62.

195. The adenovirus or adenoviral vector of claim 186, which comprises a nucleic acid sequence that is at least 96% identical to SEQ ID NO: 63.

196. The adenovirus or adenoviral vector of claim 195, which comprises the nucleic acid sequence of SEQ ID NO: 63.

197. An adenovirus or adenoviral vector comprising (1) a nucleic acid sequence encoding one or more Plasmodium antigens and (2) one or more of the nucleic acid sequences selected from the group consisting of:
(a) a nucleic acid sequence that is at least 98.4% identical to SEQ ID NO: 64,
(b) a nucleic acid sequence that is at least 99.01% identical to SEQ ID NO: 65,
(c) a nucleic acid sequence that is at least 97.08% identical to SEQ ID NO: 66,
(d) a nucleic acid sequence that is at least 96.52% identical to SEQ ID NO: 67, and
(e) a nucleic acid sequence that is at least 98.49% identical to SEQ ID NO: 68.

198. The adenovirus or adenoviral vector of claim 197, which comprises a nucleic acid sequence that is at least 98.4% identical to SEQ ID NO: 64.

199. The adenovirus or adenoviral vector of claim 198, which comprises the nucleic acid sequence of SEQ ID NO: 64.

200. The adenovirus or adenoviral vector of claim 197, which comprises a nucleic acid sequence that is at least 99.01% identical to SEQ ID NO: 65.

201. The adenovirus or adenoviral vector of claim 200, which comprises the nucleic acid sequence of SEQ ID NO: 65.

202. The adenovirus or adenoviral vector of claim 197, which comprises a nucleic acid sequence that is at least 97.08% identical to SEQ ID NO: 66.

203. The adenovirus or adenoviral vector of claim 202, which comprises the nucleic acid sequence of SEQ ID NO: 66.

204. The adenovirus or adenoviral vector of claim 197, which comprises a nucleic acid sequence that is at least 96.52% identical to SEQ ID NO: 67.

205. The adenovirus or adenoviral vector of claim 204, which comprises the nucleic acid sequence of SEQ ID NO: 67.

206. The adenovirus or adenoviral vector of claim 197, which comprises a nucleic acid sequence that is at least 98.49% identical to SEQ ID NO: 68.

207. The adenovirus or adenoviral vector of claim 206, which comprises the nucleic acid sequence of SEQ ID NO: 68.

208. An adenovirus or adenoviral vector comprising (1) a nucleic acid sequence encoding one or more Plasmodium antigens and (2) one or more of the nucleic acid sequences selected from the group consisting of:
(a) a nucleic acid sequence comprising at least 121 contiguous nucleotides of SEQ ID NO: 64,
(b) a nucleic acid sequence comprising at least 462 contiguous nucleotides of SEQ ID NO: 65,
(c) a nucleic acid sequence comprising at least 234 contiguous nucleotides of SEQ ID NO: 66,
(d) a nucleic acid sequence comprising at least 606 contiguous nucleotides of SEQ ID NO: 67, and
(e) a nucleic acid sequence comprising at least 188 contiguous nucleotides of SEQ ID NO: 68.

209. The adenovirus or adenoviral vector of claim 208, which comprises a nucleic acid sequence comprising at least 121 contiguous nucleotides of SEQ ID NO: 64.

210. The adenovirus or adenoviral vector of claim 209, which comprises the nucleic acid sequence of SEQ ID NO: 64.

211. The adenovirus or adenoviral vector of claim 208, which comprises a nucleic acid sequence comprising at least 462 contiguous nucleotides of SEQ ID NO: 65

212. The adenovirus or adenoviral vector of claim 211, which comprises the nucleic acid sequence of SEQ ID NO: 65.

213. The adenovirus or adenoviral vector of claim 208, which comprises a nucleic acid sequence comprising at least 234 contiguous nucleotides of SEQ ID NO: 66.

214. The adenovirus or adenoviral vector of claim 213, which comprises the nucleic acid sequence of SEQ ID NO: 66.

215. The adenovirus or adenoviral vector of claim 208, which comprises a nucleic acid sequence comprising at least 606 contiguous nucleotides of SEQ ID NO: 67.

216. The adenovirus or adenoviral vector of claim 215, which comprises the nucleic acid sequence of SEQ ID NO: 67.

217. The adenovirus or adenoviral vector of claim 208, which comprises a nucleic acid sequence comprising at least 188 contiguous nucleotides of SEQ ID NO: 68.

218. The adenovirus or adenoviral vector of claim 217, which comprises the nucleic acid sequence of SEQ ID NO: 68.

219. An adenovirus or adenoviral vector comprising (1) a nucleic acid sequence encoding one or more Plasmodium antigens and (2) one or more of the amino acid sequences selected from the group consisting of:
(a) an amino acid sequence that is at least 93% identical to SEQ ID NO: 69,
(b) an amino acid sequence that is at least 80% identical to SEQ ID NO: 71,
(c) an amino acid sequence that is at least 92% identical to SEQ ID NO: 72, and
(d) an amino acid sequence that is at least 88% identical to SEQ ID NO: 73.

220. The adenovirus or adenoviral vector of claim 219, which comprises an amino acid sequence that is at least 93% identical to SEQ ID NO: 69.

221. The adenovirus or adenoviral vector of claim 220, which comprises the amino acid sequence of SEQ ID NO: 69.

222. The adenovirus or adenoviral vector of claim 219, which comprises an amino acid sequence that is at least 80% identical to SEQ ID NO: 71.

223. The adenovirus or adenoviral vector of claim 222, which comprises the amino acid sequence of SEQ ID NO: 71.

224. The adenovirus or adenoviral vector of claim 219, which comprises an amino acid sequence that is at least 92% identical to SEQ ID NO: 72.

225. The adenovirus or adenoviral vector of claim 224, which comprises the amino acid sequence of SEQ ID NO: 72.

226. The adenovirus or adenoviral vector of claim 219, which comprises an amino acid sequence that is at least 88% identical to SEQ ID NO: 73.

227. The adenovirus or adenoviral vector of claim 226, which comprises the amino acid sequence of SEQ ID NO: 73.

228. An adenovirus or adenoviral vector comprising (1) a nucleic acid sequence encoding one or more Plasmodium antigens and (2) one or more of the nucleic acid sequences selected from the group consisting of:
(a) a nucleic acid sequence encoding an amino acid sequence that is at least 93% identical to SEQ ID NO: 69,
(b) a nucleic acid sequence encoding an amino acid sequence that is at least 95% identical to SEQ ID NO: 70,
(c) a nucleic acid sequence encoding an amino acid sequence that is at least 80% identical to SEQ ID NO: 71,
(d) a nucleic acid sequence encoding an amino acid sequence that is at least 92% identical to SEQ ID NO: 72, and
(e) a nucleic acid sequence encoding an amino acid sequence that is at least 88% identical to SEQ ID NO: 73.

229. The adenovirus or adenoviral vector of claim 228, which comprises a nucleic acid sequence encoding an amino acid sequence that is at least 93% identical to SEQ ID NO: 69.

230. The adenovirus or adenoviral vector of claim 229, which comprises a nucleic acid sequence encoding the amino acid sequence of SEQ ID NO: 69.

231. The adenovirus or adenoviral vector of claim 228, which comprises a nucleic acid sequence encoding an amino acid sequence that is at least 95% identical to SEQ ID NO: 70.

232. The adenovirus or adenoviral vector of claim 231, which comprises a nucleic acid sequence encoding the amino acid sequence of SEQ ID NO: 70.

233. The adenovirus or adenoviral vector of claim 228, which comprises a nucleic acid sequence encoding an amino acid sequence that is at least 80% identical to SEQ ID NO: 71.

234. The adenovirus or adenoviral vector of claim 233, which comprises a nucleic acid sequence encoding the amino acid sequence of SEQ ID NO: 71.

235. The adenovirus or adenoviral vector of claim 228, which comprises a nucleic acid sequence encoding an amino acid sequence that is at least 92% identical to SEQ ID NO: 72.

236. The adenovirus or adenoviral vector of claim 235, which comprises a nucleic acid sequence encoding the amino acid sequence of SEQ ID NO: 72.

237. The adenovirus or adenoviral vector of claim 228, which comprises a nucleic acid sequence encoding an amino acid sequence that is at least 88% identical to SEQ ID NO: 73.

238. The adenovirus or adenoviral vector of claim 237, which comprises a nucleic acid sequence encoding the amino acid sequence of SEQ ID NO: 73.

239. An adenovirus or adenoviral vector comprising (1) a nucleic acid sequence encoding one or more Plasmodium antigens and (2) one or more of the amino acid sequences selected from the group consisting of:
(a) an amino acid sequence that is at least 99% identical to SEQ ID NO: 74,
(b) an amino acid sequence that is at least 97.8% identical to SEQ ID NO: 76,
(c) an amino acid sequence that is at least 99.1% identical to SEQ ID NO: 77, and
(d) an amino acid sequence that is at least 99.2% identical to SEQ ID NO: 78.

240. The adenovirus or adenoviral vector of claim 239, which comprises an amino acid sequence that is at least 99% identical to SEQ ID NO: 74.

241. The adenovirus or adenoviral vector of claim 240, which comprises the amino acid sequence of SEQ ID NO: 74.

242. The adenovirus or adenoviral vector of claim 239, which comprises an amino acid sequence that is at least 97.8% identical to SEQ ID NO: 76.

243. The adenovirus or adenoviral vector of claim 242, which comprises the amino acid sequence of SEQ ID NO: 76.

244. The adenovirus or adenoviral vector of claim 239, which comprises an amino acid sequence that is at least 99.1% identical to SEQ ID NO: 77.

245. The adenovirus or adenoviral vector of claim 244, which comprises the amino acid sequence of SEQ ID NO: 77.

246. The adenovirus or adenoviral vector of claim 239, which comprises an amino acid sequence that is at least 99.2% identical to SEQ ID NO: 78.

247. The adenovirus or adenoviral vector of claim 246, which comprises the amino acid sequence of SEQ ID NO: 78.

248. An adenovirus or adenoviral vector comprising (1) a nucleic acid sequence encoding one or more Plasmodium antigens and (2) one or more of the nucleic acid sequences selected from the group consisting of:
(a) a nucleic acid sequence encoding an amino acid sequence that is at least 99% identical to SEQ ID NO: 74,
(b) a nucleic acid sequence encoding an amino acid sequence that is at least 99.5% identical to SEQ ID NO: 75,
(c) a nucleic acid sequence encoding an amino acid sequence that is at least 97.8% identical to SEQ ID NO: 76,
(d) a nucleic acid sequence encoding an amino acid sequence that is at least 99.1% identical to SEQ ID NO: 77, and
(e) a nucleic acid sequence encoding an amino acid sequence that is at least 99.2% identical to SEQ ID NO: 78.

249. The adenovirus or adenoviral vector of claim 248, which comprises a nucleic acid sequence encoding an amino acid sequence that is at least 99% identical to SEQ ID NO: 74.

250. The adenovirus or adenoviral vector of claim 249, which comprises a nucleic acid sequence encoding the amino acid sequence of SEQ ID NO: 74.

251. The adenovirus or adenoviral vector of claim 248, which comprises a nucleic acid sequence encoding an amino acid sequence that is at least 99.5% identical to SEQ ID NO: 75.

252. The adenovirus or adenoviral vector of claim 251, which comprises a nucleic acid sequence encoding the amino acid sequence of SEQ ID NO: 75.

253. The adenovirus or adenoviral vector of claim 248, which comprises a nucleic acid sequence encoding an amino acid sequence that is at least 97.8% identical to SEQ ID NO: 76.

254. The adenovirus or adenoviral vector of claim 253, which comprises a nucleic acid sequence encoding the amino acid sequence of SEQ ID NO: 76.

255. The adenovirus or adenoviral vector of claim 248, which comprises a nucleic acid sequence encoding an amino acid sequence that is at least 99.1% identical to SEQ ID NO: 77.

256. The adenovirus or adenoviral vector of claim 255, which comprises a nucleic acid sequence encoding the amino acid sequence of SEQ ID NO: 77.

257. The adenovirus or adenoviral vector of claim 248, which comprises an amino acid sequence that is at least 99.2% identical to SEQ ID NO: 78.

258. The adenovirus or adenoviral vector of claim 257, which comprises the amino acid sequence of SEQ ID NO: 78.

259. An adenovirus or adenoviral vector comprising (1) a nucleic acid sequence encoding one or more Plasmodium antigens and (2) one or more of the amino acid sequences selected from the group consisting of:
(a) an amino acid sequence comprising at least 89 contiguous amino acid residues of SEQ ID NO: 74,
(b) an amino acid sequence comprising at least 247 contiguous amino acid residues of SEQ ID NO: 76,
(c) an amino acid sequence comprising at least 230 contiguous amino acid residues of SEQ ID NO: 77, and
(d) an amino acid sequence comprising at least 231 contiguous amino acid residues of SEQ ID NO: 78.

260. The adenovirus or adenoviral vector of claim 259, which comprises an amino acid sequence comprising at least 89 contiguous amino acid residues of SEQ ID NO: 74.

261. The adenovirus or adenoviral vector of claim 260, which comprises the amino acid sequence of SEQ ID NO: 74.

262. The adenovirus or adenoviral vector of claim 259, which comprises an amino acid sequence comprising at least 247 contiguous amino acid residues of SEQ ID NO: 76.

263. The adenovirus or adenoviral vector of claim 262, which comprises the amino acid sequence of SEQ ID NO: 76.

264. The adenovirus or adenoviral vector of claim 259, which comprises an amino acid sequence comprising at least 230 contiguous amino acid residues of SEQ ID NO: 77.

265. The adenovirus or adenoviral vector of claim 264, which comprises the amino acid sequence of SEQ ID NO: 77.

266. The adenovirus or adenoviral vector of claim 259, which comprises an amino acid sequence comprising at least 231 contiguous amino acid residues of SEQ ID NO: 78.

267. The adenovirus or adenoviral vector of claim 266, which comprises the amino acid sequence of SEQ ID NO: 78.

268. An adenovirus or adenoviral vector comprising (1) a nucleic acid sequence encoding one or more Plasmodium antigens and (2) one or more of the nucleic acid sequences selected from the group consisting of:
(a) a nucleic acid sequence encoding an amino acid sequence comprising at least 89 contiguous amino acid residues of SEQ ID NO: 74,
(b) a nucleic acid sequence encoding an amino acid sequence comprising at least 286 contiguous amino acid residues of SEQ ID NO: 75,
(c) a nucleic acid sequence encoding an amino acid sequence comprising at least 247 contiguous amino acid residues of SEQ ID NO: 76,
(d) a nucleic acid sequence encoding an amino acid sequence comprising at least 230 contiguous amino acid residues of SEQ ID NO: 77, and
(e) a nucleic acid sequence encoding an amino acid sequence comprising at least 231 contiguous amino acid residues of SEQ ID NO: 78.

269. The adenovirus or adenoviral vector of claim 268, which comprises a nucleic acid sequence encoding an amino acid sequence comprising at least 89 contiguous amino acid residues of SEQ ID NO: 74.

270. The adenovirus or adenoviral vector of claim 269, which comprises a nucleic acid sequence encoding the amino acid sequence of SEQ ID NO: 74.

271. The adenovirus or adenoviral vector of claim 268, which comprises a nucleic acid sequence encoding an amino acid sequence comprising at least 286 contiguous amino acid residues of SEQ ID NO: 75.

272. The adenovirus or adenoviral vector of claim 271, which comprises a nucleic acid sequence encoding the amino acid sequence of SEQ ID NO: 75.

273. The adenovirus or adenoviral vector of claim 268, which comprises a nucleic acid sequence encoding an amino acid sequence comprising at least 247 contiguous amino acid residues of SEQ ID NO: 76.

274. The adenovirus or adenoviral vector of claim 273, which comprises a nucleic acid sequence encoding the amino acid sequence of SEQ ID NO: 76.

275. The adenovirus or adenoviral vector of claim 268, which comprises a nucleic acid sequence encoding an amino acid sequence comprising at least 230 contiguous amino acid residues of SEQ ID NO: 77.

276. The adenovirus or adenoviral vector of claim 275, which comprises a nucleic acid sequence encoding the amino acid sequence of SEQ ID NO: 77.

277. The adenovirus or adenoviral vector of claim 268, which comprises a nucleic acid sequence encoding an amino acid sequence comprising at least 231 contiguous amino acid residues of SEQ ID NO: 78.

278. The adenovirus or adenoviral vector of claim 277, which comprises a nucleic acid sequence encoding the amino acid sequence of SEQ ID NO: 78.

279. The adenovirus or adenoviral vector of claim 1, wherein the adenovirus or adenoviral vector requires complementation of a deficiency in one or more early regions of the adenoviral genome for propagation and does not require complementation of any other deficiency of the adenoviral genome for propagation.

280.-284. (canceled)

285. The adenovirus or adenoviral vector of claim 1, wherein the adenovirus or adenoviral vector comprises a nucleic acid sequence that encodes one Plasmodium antigen.

286. The adenovirus or adenoviral vector of claim 1, wherein the adenovirus or adenoviral vector comprises two or more nucleic acid sequences that each encode a different Plasmodium antigen.

287. The adenovirus or adenoviral vector of claim 1, wherein the Plasmodium antigen is selected from the group consisting of circumsporozoite protein (CSP), SSP1, and CelTos.

288. The adenovirus or adenoviral vector of claim 1, wherein the Plasmodium antigen is AMA-1.

289. The adenovirus or adenoviral vector of claim 1, wherein the nucleic acid sequence encoding the Plasmodium antigen comprises codons expressed most frequently in humans.

290. A composition comprising the adenovirus or adenoviral vector of claim 1 and a pharmaceutically acceptable carrier.

291. A method of inducing an immune response against Plasmodium falciparum in a mammal, which comprises administering to the mammal the adenovirus or adenoviral vector of claim 1, whereupon the nucleic acid sequence encoding the Plasmodium antigen is expressed in the mammal to produce the Plasmodium antigen and thereby induce an immune response against Plasmodium falciparum in the mammal.

292. The method of claim 291, wherein the mammal is a human.

293. A method of inducing an immune response against Plasmodium falciparum in a mammal, which comprises administering to the mammal the composition of claim 290, whereupon the nucleic acid sequence encoding the Plasmodium antigen is expressed in the mammal to produce the Plasmodium antigen and thereby induce an immune response against Plasmodium falciparum in the mammal.

294. The method of claim 293, wherein the mammal is a human.

295. The adenovirus or adenoviral vector of claim 89, wherein the adenovirus or adenoviral vector requires complementation of a deficiency in one or more early regions of the adenoviral genome for propagation and does not require complementation of any other deficiency of the adenoviral genome for propagation.

296. The adenovirus or adenoviral vector of claim 186, wherein the adenovirus or adenoviral vector requires complementation of a deficiency in one or more early regions of the adenoviral genome for propagation and does not require complementation of any other deficiency of the adenoviral genome for propagation.

297. A method of inducing an immune response against Plasmodium falciparum in a mammal, which comprises administering to the mammal the adenovirus or adenoviral vector of claim 89, whereupon the nucleic acid sequence encoding the Plasmodium antigen is expressed in the mammal to produce the Plasmodium antigen and thereby induce an immune response against Plasmodium falciparum in the mammal.

298. A method of inducing an immune response against Plasmodium falciparum in a mammal, which comprises administering to the mammal the adenovirus or adenoviral vector of claim 186, whereupon the nucleic acid sequence encoding the Plasmodium antigen is expressed in the mammal to produce the Plasmodium antigen and thereby induce an immune response against Plasmodium falciparum in the mammal.

299. A composition comprising the adenovirus or adenoviral vector of claim 89 and a pharmaceutically acceptable carrier.

300. A composition comprising the adenovirus or adenoviral vector of claim 186 and a pharmaceutically acceptable carrier.

301. A method of inducing an immune response against Plasmodium falciparum in a mammal, which comprises administering to the mammal the composition of claim 299, whereupon the nucleic acid sequence encoding the Plasmodium antigen is expressed in the mammal to produce the Plasmodium antigen and thereby induce an immune response against Plasmodium falciparum in the mammal.

302. A method of inducing an immune response against Plasmodium falciparum in a mammal, which comprises administering to the mammal the composition of claim 300, whereupon the nucleic acid sequence encoding the Plasmodium antigen is expressed in the mammal to produce the Plasmodium antigen and thereby induce an immune response against Plasmodium falciparum in the mammal.
